SEQUENCE LISTING (54 sequences)

SEQ ID NO: 1 - protein, 215 aa, Papaya mosaic virus
MSKSSMSTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLIRSPTQEERIANATNKQVHLFQAAAQDNNFTSNSAFITKGQISGSTPTIQFLPPPE

SEQ ID NO: 2 - DNA, 648 bp, Papaya mosaic virus
atgtctaagtcaagtatgtccacacccaacatagccttccccgccatcacccaggaacagatgagctcgattaaggtcgatccaacgtccaatcttctgccctcccaagagcagttaaagtcagtgtccaccctcatggtagctgctaaggttccagcagccagtgttacaactgtggcattggagttggtcaacttctgctatgacaatgggtccagcgcgtacaccacagtgactggcccatcatcaataccggagatatcactggcacaattggctagtattgtcaaagcttccggcacttcccttagaaaattctgccggtacttcgcgccaataatctggaatctgaggacggacaaaatggctcctgccaattgggaggcttcaggatacaagccaagcgccaaatttgccgcgttcgacttcttcgacggggtggagaatccggcggccatgcaacccccttcgggactaatcaggtcgccgacccaggaagagcggattgccaatgctaccaacaaacaggtgcatctcttccaagccgcggcacaggacaacaactttaccagcaactccgccttcatcaccaaaggccaaatttctgggtcaaccccaaccatccaattccttccaccccccgaataa

SEQ ID NO: 3 - protein, 211 aa, Artificial Sequence
  modified PapMV coat protein CPdeltaN5
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPE

SEQ ID NO: 4 - protein, 10 aa, Artificial Sequence
  loop 6 peptide from Salmonella typhi porin
GTSNGSNPST

SEQ ID NO: 5 - protein, 14 aa, Artificial Sequence
  loop 7 peptide from Salmonella typhi porin
KDISNGYGASYGDQ

SEQ ID NO: 6 - protein, 8 aa, Artificial Sequence
  Enterobacterial porin epitope sequence
TRVAFAGL

SEQ ID NO: 7 - protein, 8 aa, Artificial Sequence
  Enterobacterial porin epitope sequence
RNTDFFGL

SEQ ID NO: 8 - protein, 8 aa, Artificial Sequence
  Enterobacterial porin epitope sequence
RNSDFFGL

SEQ ID NO: 9 - protein, 8 aa, Artificial Sequence
  Enterobacterial porin epitope sequence
TRLAFAGL

SEQ ID NO: 10 - protein, 8 aa, Artificial Sequence
  Enterobacterial porin epitope sequence
TRLGFAGL

SEQ ID NO: 11 - protein, 7 aa, Artificial Sequence
  Enterobacterial porin epitope sequence
SDDKGSD

SEQ ID NO: 12 - protein, 9 aa, Artificial Sequence
  Enterobacterial porin epitope sequence
NQTEGSNDS

SEQ ID NO: 13 - protein, 34 aa, Artificial Sequence
  Enterobacterial porin epitope sequence
NYGVTYDVTSWTDVLPEFGGDTYGADNFMQQRGN

SEQ ID NO: 14 - protein, 16 aa, Artificial Sequence
  Enterobacterial porin epitope sequence
GSVSGENTNGRSLLNQ

SEQ ID NO: 15 - protein, 19 aa, Artificial Sequence
  Enterobacterial porin epitope sequence
RTADQNNTANARLYGNGDR

SEQ ID NO: 16 - protein, 21 aa, Artificial Sequence
  Enterobacterial porin epitope sequence
NATRFGTSNGSNPSTSYGFAN

SEQ ID NO: 17 - protein, 17 aa, Artificial Sequence
  Enterobacterial porin epitope sequence
DISNGYGASYGDQDIVK

SEQ ID NO: 18 - protein, 18 aa, Artificial Sequence
  Enterobacterial porin epitope sequence
NLLDKNDETRDAGINTDD

SEQ ID NO: 19 - protein, 8 aa, Artificial Sequence
  Enterobacterial porin epitope sequence
TRXXFAGL

SEQ ID NO: 20 - protein, 8 aa, Artificial Sequence
  Enterobacterial porin epitope sequence
RNXDFFGL

SEQ ID NO: 21 - protein, 7 aa, Artificial Sequence
  affinity peptide
SLSLIQT

SEQ ID NO: 22 - protein, 7 aa, Artificial Sequence
  affinity peptide
EAKGLIR

SEQ ID NO: 23 - protein, 7 aa, Artificial Sequence
  affinity peptide
TATYLLD

SEQ ID NO: 24 - protein, 7 aa, Artificial Sequence
  affinity peptide
FHENWPS

SEQ ID NO: 25 - protein, 7 aa, Artificial Sequence
  affinity peptide
FHEFWPT

SEQ ID NO: 26 - protein, 7 aa, Artificial Sequence
  affinity peptide
FHEXWPT

SEQ ID NO: 27 - DNA, 30 bp, Artificial Sequence
  primer
agtcccatggatccaacgtccaatcttctg

SEQ ID NO: 28 - DNA, 49 bp, Artificial Sequence
  primer
atgcggatccttactaatggtgatggtgatggtgttcggggggtggaag

SEQ ID NO: 29 - DNA, 63 bp, Artificial Sequence
  oligonucleotide
ctagttcttctgcgttcaccatcatggaccaggttccgttctctgtttctgtttctcagctga

SEQ ID NO: 30 - DNA, 63 bp, Artificial Sequence
  oligonucleotide
ctagtcagctgagaaacagaaacagagaacggaacctggtccatgatggtgaacgcagaagaa

SEQ ID NO: 31 - DNA, 63 bp, Artificial Sequence
  oligonucleotide
ctagttctccgctgaccaaaggtatcctgggtttcgttttcaccctgaccgttccgtctgaaa

SEQ ID NO: 32 - DNA, 63 bp, Artificial Sequence
  oligonucleotide
ctagtttcagacggaacggtcagggtgaaaacgaaacccaggatacctttggtcagcggagaa

SEQ ID NO: 33 - DNA, 80 bp, Artificial Sequence
  oligonucleotide
gatcactagtgtggtggtgggtaccaccgatcgtagcggtgcgccgacctacagctggggtgcgaacgatacgcgtcatg

SEQ ID NO: 34 - DNA, 80 bp, Artificial Sequence
  oligonucleotide
catgacgcgtatcgttcgcaccccagctgtaggtcggcgcaccgctacgatcggtggtacccaccaccacactagtgatc

SEQ ID NO: 35 - DNA, 33 bp, Artificial Sequence
  primer
agtcccatggccgatccaacgtccaatcttctg

SEQ ID NO: 36 - DNA, 28 bp, Artificial Sequence
  primer
acgtccatggtatatctccttcttaaag

SEQ ID NO: 37 - DNA, 45 bp, Artificial Sequence
  primer
atcgccatggcatccacacccaacatagccttccccgccatcacc

SEQ ID NO: 38 - DNA, 76 bp, Artificial Sequence
  primer
ggttaaggaaggtggggggcttctccgcttccccaactaagcatggtagtggtagtggtaatcattcctaggtgac

SEQ ID NO: 39 - protein, 378 aa, Salmonella Typhi Ty2
MKVKVLSLLVPALLVAGAANAAEIYNKDGNKLDLFGKVDGLHYFSDDKGSDGDQTYMRIGFKGETQVNDQLTGYGQWEYQIQGNQTEGSNDSWTRVAFAGLKFADAGSFDYGRNYGVTYDVTSWTDVLPEFGGDTYGADNFMQQRGNGYATYRNTDFFGLVDGLDFALQYQGKNGSVSGENTNGRSLLNQNGDGYGGSLTYAIGEGFSVGGAITTSKRTADQNNTANARLYGNGDRATVYTGGLKYDANNIYLAAQYSQTYNATRFGTSNGSNPSTSYGFANKAQNFEVVAQYQFDFGLRPSVAYLQSKGKDISNGYGASYGDQDIVKYVDVGATYYFNKNMSTYVDYKINLLDKNDFTRDAGINTDDIVALGLVYQF

SEQ ID NO: 40 - protein, 363 aa, Salmonella Typhi CT18
MMKRKILAAVIPALLAAATANAAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMSIVENTVTDTVEMANKTQNLEVVAQYQFDFGLRPAISYVQSKGKQLNGADGSADLAKYIQAGATYYFNKNMNVWVDYRFNLLDENDYSSSYVGTDDQAAVGITYQF

SEQ ID NO: 41 - DNA, 77 bp, Artificial Sequence
  primer
ggttaaggaaggtggggggcttaaagtactcttaaccggaagcgtggtagtggtagtggtaatcattcctaggtgac

SEQ ID NO: 42 - protein, 224 aa, Artificial Sequence
  PapMV coat protein comprising an affinity peptide for binding to OmpC
MASTPNIAFPAITQEQMSSIEVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQGNNFASNSAFITKGQISGSTPTIQFLPPPEEAKGLIRHHHHHH

SEQ ID NO: 43 - protein, 224 aa, Artificial Sequence
  PapMV coat protein comprising an affinity peptide for binding to OmpF
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPEFHENWPSHHHHHH

SEQ ID NO: 44 - DNA, 54 bp, Artificial Sequence
  primer
ctagtggtacttctaacggttctaacccgtctacttcttacggtttcgcgaaca

SEQ ID NO: 45 - DNA, 54 bp, Artificial Sequence
  primer
ctagtgttcgcgaaaccgtaagaagtagacgggttagaaccgttagaagtacca

SEQ ID NO: 46 - protein, 237 aa, Artificial Sequence
  PapMV coat protein comprising the S. typhi porin loop 6 peptide
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSGTSNGSNPSTSYGFANTRHHHHHH

SEQ ID NO: 47 - DNA, 714 bp, Artificial Sequence
  nucleotide sequence encoding PapMV coat protein comprising the S. typhi porin loop 6 peptide
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaactttgccagcaactccgccttcatcaccaaaggccaaatttctgggtcaaccccaaccatccaattccttccaccccccgaaactagtggtacttctaacggttctaacccgtctacttcttacggtttcgcgaacacgcgtcaccatcaccatcaccattag

SEQ ID NO: 48 - protein, 228 aa, Artificial Sequence
  PapMV SM coat protein comprising an affinity peptide for binding to OmpC
MASTPNIAFPAITQEQMSSIKVDPTSNLLPSQEQLKSVSTLMVAAKVPAASVTTVALELVNFCYDNGSSAYTTVTGPSSIPEISLAQLASIVKASGTSLRKFCRYFAPIIWNLRTDKMAPANWEASGYKPSAKFAAFDFFDGVENPAAMQPPSGLTRSPTQEERIANATNKQVHLFQAAAQDNNFASNSAFITKGQISGSTPTIQFLPPPETSEAKGLIRTRHHHHHH

SEQ ID NO: 49 - DNA, 687 bp, Artificial Sequence
  nucleotide sequence encoding PapMV SM coat protein comprising an affinity peptide for binding to OmpC
atggcatccacacccaacatagccttccccgccatcacccaggaacaaatgagctcgattaaggtcgatccaacgtccaatcttctgccctcccaagagcagttaaagtcagtgtccaccctcatggtagctgctaaggttccagcagccagtgttacaactgtggcattggagttggttaacttctgctatgacaatgggtccagcgcgtacaccacagtgactggcccatcatcaataccggagatatcactggcacaattggccagcattgtcaaagcttccggcacttcccttaggaaattctgccggtacttcgcgccaataatctggaatctgaggacggacaaaatggctcctgccaattgggaggcctcaggatacaagccaagcgccaaatttgccgcgttcgacttcttcgacggggtggagaatccggcggccatgcaacccccttcgggactaaccaggtcgccgacccaggaagagcggattgccaatgccaccaacaaacaggtgcatctcttccaagccgcggcacaggacaacaactttgccagcaactccgccttcatcaccaaaggccaaatttctgggtcaaccccaaccatccaattccttccaccccccgaaactagtgaggcgaaggggttgattcgtacgcgtcaccatcaccatcaccattag

SEQ ID NO: 50 - protein, 16 aa, Artificial Sequence
  peptide comprising loop 6 epitope from Salmonella typhi porin
GTSNGSNPSTSYGFAN

SEQ ID NO: 51 - protein, 9 aa, Artificial Sequence
  epitope from gp100 tumor antigen
IMDQVPFSV

SEQ ID NO: 52 - protein, 9 aa, Artificial Sequence
  epitope from influenza M1 protein
GILGFVFTL

SEQ ID NO: 53 - protein, 9 aa, Artificial Sequence
  epitope from gp100 tumor antigen
ITDQVPFSV

SEQ ID NO: 54 - protein, 19 aa, Artificial Sequence
  Loop 7 peptide from Salmonella typhi porin
QSKGKDISNGYGASYGDQD

(19) United States
(12) Patent Application Publication
(10) Pub. No.: US 2010/0316665 A1
(43) Pub. Date: Dec. 16, 2010

(54) PAPAYA MOSAIC VIRUS-BASED VACCINES AGAINST SALMONELLA TYPHI AND OTHER ENTEROBACTERIAL PATHOGENS

(76) Inventors: Denis Leclerc, Quebec (CA); Constantino Roberto López-Macias, III, Mexico (MX)

Correspondence Address: K&L Gates LLP, 3580 Carmel Mountain Road, Suite 200, San Diego, CA, 92130, US

(21) Appl. No.: 12/449,140

(22) PCT Filed: Jan. 28, 2008
(86) PCT No.: PCT/CA2008/000154

§ 371 Date: Jan. 6, 2010

Related U.S. Application Data
(60) Provisional application No. 60/886,873, filed on Jan. 26, 2007.

(51) Int. Cl. A61K 39/385; A61K 39/295; C07K 14/00; C07H 21/00; A61P 31/12; A61P 37/04

(52) U.S. Cl. 424/196.11; 424/201.1; 530/350; 536/23.72

(57) ABSTRACT

An antigen-presenting system (APS) comprising one or more enterobacterial antigens in combination with a papaya mosaic virus (PapMV) or a virus like particle (VLP) derived from papaya mosaic virus is provided. The PapMV or VLP included in the APS is capable of potentiating an immune response against said one or more enterobacterial antigens. The APS can be used, for example, as a vaccine against enterobacterial disease, such as typhoid fever. The one or more antigens comprised by the APS can be conjugated to a coat protein of the PapMV or PapMV VLP, or they may be non-conjugated (i.e. separate from the PapMV or PapMV VLP), or the APS can comprise both conjugated and non-conjugated antigens. Conjugation can be, for example, by genetic fusion with the coat protein, or binding via covalent, non-covalent or affinity means.

### FIELD OF THE INVENTION

The present invention relates to the field of vaccine formulations and adjuvants and, in particular, to vaccines based on plant virus particles.

### BACKGROUND OF THE INVENTION

The Enterobacteriaceae comprise a large family of gram-negative bacteria and include numerous pathogens of clinical importance to human and veterinary medicine. Enterobacterial pathogens are the causative agents of both intestinal and systemic infections and include Salmonella enterica (typhoid fever and foodborne gastroenteritis), pathogenic Escherichia coli (various foodborne intestinal infections, urinary tract infections, meningitis, and sepsis), Shigella spp. (bacillary dysentery), Yersinia spp. (plague and enterocolitis), and Klebsiella and Enterobacter spp. (pneumonia and blood stream infections). These enterobacterial species are also associated with hospital-acquired infections and gram-negative sepsis. Furthermore, multiresistance to antibiotics has become a major issue in the treatment of diseases caused by enterobacteria (Paterson, 2006, AJIC 34:20-28).

At least five types of gastrointestinal disease in humans are caused by E. coli. Pathogenicity in E. coli strains is due to the presence of one or more virulence factors. These include: invasiveness factors (invasins), heat-labile and heat-stable enterotoxins, verotoxins, and colonization factors or adhesins. The most recently identified E. coli disease is hemorrhagic colitis which is caused by strains of serotype 0157:H7. These strains produce verotoxin, and the disease is characterized by painful abdominal cramping and bloody diarrhea. The same strains are associated with hemolytic uremic syndrome (HUS) in humans.

Yersinia enterocolitica also causes diarrhea in humans. In addition, diarrhea has been associated with infection caused by Edwardsiella tarda and Citrobacter strains. Strains of Klebsiella pneumoniae and Enterobacter cloacae have been isolated from patients with tropical sprue.

Typhoid fever, a serious systemic infection, is caused by an acute infection of the reticuloendothelial system with the enterobacterium Salmonella enterica serovar Typhi (S. typhi)). At least 16 million cases of typhoid arise each year, resulting in 600,000 deaths. The infection is spread by the fecal-oral route and usually enters the body by consumption of contaminated food or water. After it penetrates the intestinal wall, it multiplies and enters the blood stream within 24-72 hours resulting in enteric fever and bacteremia. Within a period of 10 to 14 days, early symptoms of typhoid include headache, fever, constipation, stomach pains, anorexia and myalgia.

The Enterobacteriaceae family also represents a major group of pathogens in non-human animals. For example, Klebsiella pneumoniae is a frequent cause of respiratory disease in primates, and Yersinia pseudotuberculosis is associated with enterocolitis and peritonitis. The most frequently diagnosed diarrheal diseases in primates are caused by Shigella, E. coli and Salmonella. Domestic pets such as cats and dogs are susceptible to diseases such as cystitis and other urogenital infections, all of which are caused by E. coli. Proteus species cause other diseases in cats and dogs.

Farm animals such as calves are susceptible to both systemic colibacillosis and neonatal diarrhea (calf scours), which are caused by heat-stable enterotoxigenic E. coli serotypes containing K99 fimbrial adhesin. Bovine mastitis is most often caused as a result of infection with E. coli and Serratia species, and less often, Klebsiella species and Citrobacter freundii. Salmonella dublin and S. typhimurium are also important pathogens in cows. Infection with E. coli causes diarrhea in piglets, or edema preceded by mild diarrhea. Swine E. coli strains are characterized by the presence of K88 fimbrial adhesin, which is antigenically distinct from the above-noted K99 antigen. Mastitis and metritis in swine is caused by K. pneumoniae, and enteritis and lymphadenitis caused by Yersinia enterocolitica. S. choleraesuis and S. typhisuis also cause disease in pigs.

In horses, Salmonellae spp., such as S. typhimurium, S. newport, and S. anatum, cause enteritis which results in high fatality and septic abortion. Foals and mares fall victim to K. pneumoniae infections which cause metritis (mares) and pneumonia (foals). Sheep also suffer from a variety of illnesses caused by Enterobacteriaceae. Strains of E. coli producing a heat-stable enterotoxin, for example, cause infant diarrhea in lambs. Most of these strains contain the K-99 fimbrial adhesin. Salmonella abortion is usually caused by Salmonella abortusovis, S. typhimurium, or S. dublin, which may also result in stillbirths and wool damage.

In chickens, septicemia caused by E. coli is an important cause of death. Serotypes of Salmonella enterica as well as Salmonella pullorum and Salmonella gallinarum are also important pathogens in domestic fowl.

Substantial losses in the fishing industry can also be attributed to infection by various enterobacteria. Outbreaks of redmouth disease in salmon and trout hatcheries, for example, are caused by Yersinia ruckeri. Edwardsiella tarda is pathogenic for eels, catfish, and goldfish, and Edwardsiella ictaluri is pathogenic for catfish.

While antibiotic treatment of enterobacterial infections are available, the emergence and spread of resistance in Enterobacteriaceae are complicating the treatment of infectious disease and threatening to create species resistant to all currently available agents (Paterson, 2006, AJIC 34:20-28). As such, development of vaccines for the prophylactic and therapeutic treatment of bacterial infection has been investigated. U.S. patent application Ser. No. 10/394,517 (published as 2003/0215464), for example, describes attenuated Enterobacteriaceae bacteria that lack a functional lipoprotein (LPS). These attenuated bacteria are unable to induce an immune response and thus are potential vaccine candidates.

Vaccines against typhoid fever have also been developed. The oral live attenuated galE mutant Ty21a (Vivotif® vaccine; Bema Biotech, Ltd., Berne, Switzerland) is effective in endemic areas, but it is not licensed for use in children younger than six years old. Also, three to four doses are required to reach a partial protective immune response (Levine et al., 1999, Vaccine 1: S22-S27). Vi capsular polysaccharide vaccine (ViCPS) (Typhim Vi™; Aventis Pasteur) is licensed for children over 2 years old; one injection of Vi provides similar protection to the Ty21a vaccine, but only for a short period. The lack of long lasting immunity is the major disadvantage of this vaccine (Lin et al., 2001, N Engl J Med 344:1263-9; Sabitha et al., 2004, Indian J. Med. Sci. 58:141-149). Furthermore, the best protection achieved for these vaccines typically ranges between 50% and 80% of recipients (Crump et al., 2004, Bulletin of the World Health Organisation, 82; 346-353; Levine et al., 1999, Vaccine 1: S22-S27; Lin et al., 2001, N Engl J Med 344:1263-9; Sabitha et al., 2004, Indian J. Med. Sci. 58:141-149). A new candidate vaccine based on an attenuated strain of S. typhi (Ty-800) has been developed by Emergent BioSolutions, Inc. (Gaithersburg, Md.). This vaccine candidate is currently in phase II clinical trials in Vietnam, an endemic area (Crump et al., 2004, ibid.). As with the Ty21a vaccine, this vaccine is administered by the oral route and will likely not be suitable for administration to children.

Although the potential broad use of attenuated bacteria as a vaccine or vaccine vector for the prevention and treatment of infectious disease has significant advantages over other vaccines, safety issues concerning the use of attenuated bacteria are not trivial. The development of alternative treatments for infectious diseases, including novel vaccine platforms in combination with specific antigens are, therefore, considered a priority for global health improvement over the next 5-10 years (Nikaido, 2003, Microbiol Mol Biol Rev. 67:593-656). In this regard, studies of the human immune response to superficial bacterial antigens have already been reported. The ability of Vi (capsular polysaccharide), H (flagellar component) and O (lipopolysaccharide [LPS]) bacterial antigens to elicit an antibody response against S. typhi, for example, provides evidence that the development of alternative regimes using specific antigens is worthy of pursuit (discussed in Calderon et al., 1986, Infection and Immunity, 52:209-212; Ortiz, et al., 1989, J Clin. Microbiol. 27:1640-1645). More recently, researchers have found that porins are also important antigens for the induction of specific protective immune responses against infection caused by several gram-negative bacteria (Humphries et al., 2006, Vaccine 24:36-44; Kim et al., 1999, J Immunol. 162:6855-6866). Porins are trimeric exposed outer membrane proteins (OMPs) of gram-negative bacteria that function as relatively nonspecific channels, allowing small hydrophilic molecules to pass across the outer membrane (Nikaido, 2003, Microbiol Mol Biol Rev. 67:593-656).

Immune responses to porins appear to involve both the humoral and cell-mediated immune pathways. Typhoid fever acute and convalescent patients, for example, show high levels of porin-specific antibodies (Calderon et al., 1986, Infect. Immun. 52:209-212, Ortiz, et al., 1989, J Clin. Microbiol. 27:1640-1645). In addition, typhoid fever patients and human volunteers immunized with Ty21a oral vaccine have shown porin-specific cellular immune responses (Salerno-Goncalves, et al., 2002, J Immunol. 169:2196-203). OmpC and OmpF, two key S. typhi porins, have been shown to raise a long-lasting antibody response in mice (Secundino et al., 2006, Immunology 117:59). Thus, taken together, these data indicate that porins are important targets for both cellular and humoral immune responses in respect of enterobacterial infections.

Among the numerous new approaches to vaccine development, virus-like-particles (VLPs) made of viral nucleocapsids have emerged as a promising strategy. To date, two VLP vaccines, hepatitis B virus (HBV) and Human Papilloma Virus (HPV), have been shown to function efficiently in humans (Fagan et al., 1987, J. Med Virol., 21:49-56; Harper et al., 2004, Lancet, 364:1757-1765). VLPs made from the human papillomavirus (HPV) major capsid protein L1, for example, were shown to provide 100% protection in woman against development of cervical cancers (Ault, K. A., 2006, Obstet. Gynecol. Surv. 61:S26-S31; Harper et al., 2004, Lancet 364:1757-1765, see also International Patent Application PCT/US01/18701 (WO 02/04007)). Platforms such as the bacteriophage Qβ (Maurer et al., 2005, Eur. J. Immunol. 35:2031-2040), the hepatitis B virus VLPs made of the viral core protein (Mihailova et al., 2006, Vaccine 24:4369-4377; Pumpens et al., 2002, Intervirology 45:24-32), and parvorirus VLPs (Antonis et al., 2006, Vaccine 24:5481-5490; Ogasawara et al., 2006, In Vivo 20:319-324) have also shown capacity to carry epitopes and induce a strong antibody response. Similarly, U.S. Pat. No. 6,627,202, describes HBV core proteins comprising antigens crosslinked by HBV capsid-binding peptides for use as epitope delivery systems, including antigens targeted to or derived from various viruses and bacteria.

The use of VLPs from plant viruses as epitope presentation systems has been described. Plant viruses are comprised mainly of proteins that are highly immunogenic, and possess a complex, repetitive and crystalline organisation. In addition, they are phylogenetically distant from the animal immune system, which makes them good candidates for the development of vaccines. For example, cowpea mosaic virus (CPMV), Johnson grass mosaic virus (JGMV), tobacco mosaic virus X (TMVX), and alfalfa mosaic virus (AIMV) have been modified for the presentation of epitopes of interest (Canizares, M. C. et al., 2005, Immunol. Cell. Biol. 83:263-270; Brennan et al., 2001, Molec. Biol. 17:15-26; Saini and Vrati, 2003, J. Virol. 77:3487-3494). International Patent Application PCT/GB97/01065 (WO 97/39134) describes chimaeric virus-like particles that comprise a coat protein and a non-viral protein, which can be used, for example, for presentation of peptide epitopes. International Patent Application PCT/US01/07355 (WO 01/66778) describes a plant virus coat protein, and specifically a tobamovirus coat protein, fused via a linker at the N-terminus to a polypeptide of interest, which may include an epitope of a pathogenic microorganism. International Patent Application PCT/US01/20272 (WO 02/00169) describes vaccines comprising either potato virus Y coat protein or a truncated bean yellow mosaic virus coat protein fused to a foreign peptide, and specifically a Newcastle Disease Virus or human immunodeficiency virus (HIV) epitope. Also, U.S. Pat. No. 6,042,832 describes methods of administering fusions of polypeptides, such as pathogen epitopes, with alfalfa mosaic virus or ilarvirus capsid proteins to an animal in order to raise an immune response.

VLPs derived from the coat protein of papaya mosaic virus (PapMV) and their use as immunopotentiators has been described (International Patent Application No. PCT/CA03/00985 (WO 2004/004761)). Expression of the PapMV coat protein in E. coli leads to the self-assembly of VLPs composed of several hundred CP subunits organised in a repetitive and crystalline manner (Tremblay et al., 2006, FEBS J 273:14). Studies of the expression and purification of PapMV CP deletion constructs further indicate that self-assembly (or multimerization) of the CP subunits is important for function (Lecours et al., 2006, Protein Expression and Purification, 47:273-280). The ability of PapMV VLPs comprising epitopes from either gp100 or the influenza virus M1 protein have been shown to induce MHC class I cross-presentation of the epitopes leading to expansion of specific human T cells (Leclerc, D., et al., J. Virol, 2007, 81(3):1319-26; Epub. ahead of print Nov. 22, 2006).

VLPs derived from Potato Virus X (PVX) carrying various antigenic determinant from HIV, HCV, EBV or the influenza virus have been described (European Patent Application No. 1 167 530). The ability of the PVX VLP carrying an HIV epitope to induce antibody production in mice via humoral and cell-mediated pathways. Additional adjuvants were used in conjunction with the PVX VLP to potentiate this effect.

Hepatitis B core protein or parvovirus VLPs have been reported to induce a CTL response even when they do not carry genetic information (Ruedl et al., 2002, Eur. J. Immunol. 32; 818-825; Martinez et al., 2003, Virology, 305; 428-435) and can not actively replicate in the cells where they are invaginated. The cross-presentation of such VLPs carrying an epitope from lymophocytic choriomeningitis virus (LCMV) or chicken egg albumin by dendritic cells in vivo has also been described (Ruedl et al., 2002, ibid; Morón, et al., 2003, J. Immunol. 171:2242-2250). The ability of a hepatitis B core protein VLP carrying an epitope from LCMV to prime a CTL response has also been described, however, this VLP was unable to induce the CTL response when administered alone and failed to mediate effective protection from viral challenge. An effective CTL response was induced only when the VLP was used in conjunction with anti-CD40 antibodies or CpG oligonucleotides (Storni, et al., 2002, J. Immunol. 168:2880-2886). An earlier report indicated that porcine parvovirus-like particles (PPMV) carrying a peptide from LCMV were able to protect mice against a lethal LCMV challenge (Sedlik, et al., 2000, J. Virol. 74:5769-5775).

The use of papaya mosaic virus VLPs fused to affinity peptides capable of binding resting spores of Plasmodiophora brassicae has also been described (Morin et al., 2007, J. Biotechnology, 128: 423-434). The VLPs were shown to be capable of binding P. brassacae spores with high avidity and were proposed as an alternative to antibodies for the detection of P. brassacae.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide papaya mosaic virus-based vaccines against Salmonella typhi and other enterobacterial pathogens. In accordance with one aspect of the present invention, there is provided an antigen-presenting system comprising one or more enterobacterial antigens in combination with a papaya mosaic virus (PapMV) or a VLP derived from a PapMV coat protein, wherein said PapMV or VLP is capable of potentiating an immune response against said one or more enterobacterial antigens.

In accordance with another aspect of the present invention, there is provided a vaccine composition comprising one or more antigen-presenting systems of the invention and a pharmaceutically acceptable carrier.

In accordance with another aspect of the present invention, there is provided a polypeptide comprising a papaya mosaic virus coat protein fused to one or more affinity peptides capable of binding an enterobacterial antigen.

In accordance with another aspect of the present invention, there is provided a polypeptide comprising a papaya mosaic virus coat protein fused to one or more enterobacterial antigens.

In accordance with another aspect, there is provided a polynucleotide encoding a polypeptide of the invention.

In accordance with another aspect, there is provided a use of an antigen presenting system of the invention for inducing an immune response against an enterobacterium in an animal in need thereof.

In accordance with another aspect, there is provided a use of a vaccine of the invention for inducing an immune response against an enterobacterium in an animal in need thereof.

In accordance with another aspect, there is provided a use of an antigen presenting system of the invention in the preparation of a vaccine.

In accordance with another aspect of the present invention, there is provided a vaccine for immunizing an animal against infection with Salmonella typhi, said vaccine comprising an antigen-presenting system comprising an antigen derived from Salmonella typhi OmpC or OmpF in combination with a papaya mosaic virus (PapMV) or a VLP derived from a PapMV coat protein, wherein said PapMV or VLP is capable of potentiating an immune response against said antigen in the animal.

In accordance with another aspect of the present invention, there is provided a method of inducing an immune response against an enterobacterium in an animal, said method comprising administering to said animal an effective amount of an antigen presenting system of the invention.

In accordance with another aspect of the present invention, there is provided a method of inducing an immune response against an enterobacterium in an animal, said method comprising administering to said animal an effective amount of the vaccine of the invention.

### BRIEF DESCRIPTION OF THE DRAWINGS

These and other features of the invention will become more apparent in the following detailed description in which reference is made to the appended drawings.

FIG. 1 presents (A) the amino acid sequence for the papaya mosaic virus coat (or capsid) protein (GenBank Accession No. NP_{—}044334.1; SEQ ID NO:1), (B) the nucleotide sequence encoding the papaya mosaic virus coat protein (GenBank Accession No. NC_{—}001748 (nucleotides 5889-6536); SEQ ID NO:2), and (C) the amino acid sequence of the modified PapMV coat protein. CPΔN5 (SEQ ID NO:3).

FIG. 2 presents electron micrographs, in which the bars indicate 200 nm, of (A) WT PapMV virus, (B) CPΔN5 PapMV virus-like particles (VLPs), (C) E128A VLPs, (D) K97A purified protein, (E) the immunogold labelling of recombinant CPΔN5 VLPs with antibodies raised against the 6× H tag, and F) the average length of PapMV virus and the recombinant VLPs CPΔN5 and E128A (n=150).

FIG. 3 presents circular dichroism (CD) spectra of wild-type (WT) PapMV and PapMV virus-like particles (VLPs) comprising the recombinant mutant PapMV coat proteins CPΔN5, E128A or K97A; (A) shows far-UV spectra of WT virus (black line), CPΔN5 (dotted line) and E128A VLPs (grey line), (B) shows far-UV spectra of isolated disks from the WT virus by the acetic acid method (black line) and high speed supernatant of the CPΔN5 protein (disks) (dotted line), (C) shows far-UV spectra of the CPΔN5 protein (disks) (dotted line) and the K97A protein (grey line), (D) shows far-UV spectra of the CPΔN5 and E128A VLPs between 250 and 350 nm, and (E) shows far-UV spectra of the CPΔN5 and K97A disks between 250 and 350 nm.

FIG. 4 presents the results of a gel filtration analysis of the recombinant mutant PapMV coat proteins, CPΔN5 and K97A; (A) depicts the protein elution profile for the CPΔN5 purified protein (black line; grey line: molecular weight markers), and (B) depicts the protein elution profile for the CPΔN5 purified protein (black line) and the K97A purified protein (grey line).

FIG. 5 presents circular dichroism (CD) spectra temperature-induced denaturation curves for the recombinant mutant and wild-type (WT) PapMV coat proteins; (A) shows the profiles for CPΔN5 and E128A mutant VLPs and WT virus, and (B) shows the profiles for CPΔN5 and WT disks. Spectra are presented in units of mean residue ellipticity. Unfolding was monitored by recording [Q] at 222 nm as a function of temperature. All CD spectra shown were generated with proteins at a concentration of 1 mg/ml in 10 mM NaP buffer pH 7.2.

FIG. 6 presents (A) an alignment of the C-terminal sequences of PapMV coat protein (PapMV), and modified PapMV constructs containing HLA-A*0201 restricted epitopes from Influenza M1 protein (position 57 to 65; designated PapMV Flu) or from gp100 (position 209-217 with an M in position 210; designated PapMV gp100), and (B) electron microscope images of each of the recombinant auto-assembled PapMV preparations.

FIG. 7 presents the results of SDS-PAGE analysis of the stability of recombinant PapMV coat protein (PapMV) and coat protein fusions (PapMV-gp100 and PapMV-Flu) 2 and 7 months after their synthesis. Also illustrated are the results of incubating 7 month old protein preparations for 7 additional days at 23 or 37° C., and treatment with proteinase K for 5 minutes prior to addition of loading buffer and incubation in boiling water.

FIG. 8 illustrates that MHC class I epitopes from both antigens can be cross-presented when pulsed on 2 different sources of APC. CD40-activated B lymphocytes and DC were prepared from an HLA-A*0201 donor and pulsed for 18 h with indicated preparations at various concentrations. Pulsed cells were washed and gp100-(A) or FLU-(B) specific T cells were added for additional 18 h. Evidence of T cells activation was revealed by IFN-γ secretion determined by ELISA assay.

FIG. 9 illustrates the results from (A) co-culturing PapMV Flu or PapMV gp100 pulsed CD40-activated B lymphocytes prepared from HLA-A*02 positive or negative donors with FLU-(left panel) or gp100 (right panel)-specific T cells, and (B) incubating an HLA-A*0201⁺ and gp100⁺ melanoma line, or PapMV Flu pulsed HLA-A*02⁺ CD40-activated B cells with antibodies blocking MHC class I, class II or HLA-DR presentation. FLU- or gp100-specific T cells were then added. Results are represented as percentage of recognition based on IFN-γ secretion assay, 100% corresponding to the amount secreted by positive controls.

FIG. 10 illustrates that MHC class I cross-presentation mediated by PapMV is proteasome independent. An HLA-A*0201⁺ and gp100⁺ melanoma line, or PapMV Flu-pulsed HLA-A*02⁺ CD40-activated B cells were incubated with antibodies blocking MHC class I, class II or HLA-DR presentation. FLU- or gp100-specific T cells were then added. Results are represented as percentage of recognition based on IFN-γ secretion assay, 100% corresponds to the amount secreted by positive controls.

FIG. 11 presents an analysis of expansion of T lymphocytes specific to the HLA-A*0201 epitope from Influenza M1 protein with PapMV Flu-pulsed APC. (A) PBMC from an HLA-A2⁺ normal donor were co-cultured with autologous CD40-activated B cells (mock) or CD40-activated B cells pulsed with either PapMV, PapMV Flu or PapMV gp100. Cultured cells were restimulated on day 7 and IL-2 was added at different times. Specificity of expanded cells was assessed on day 15 by co-culture with T2 cells pulsed with HLA-A*0201 peptides from Flu or gp100. The frequency of IFN-γ secreting cells was determined by ELISPOT. (B) Expanded cultured T cells were co-cultured with pulsed CD40-activated B lymphocytes.

FIG. 12 presents the results of an analysis of IFN-γ secretion of expanded T cells treated as for FIG. 11.

FIG. 13 presents a comparison of the pro-inflammatory response of PapMV coat protein and LPS in dorsal air pouches raised in CD-1 mice. Data represent the mean±SEM of 5 mice. The results are representative of two identical and independent experiments.

FIG. 14 illustrates the IgG antibody response specific for the PapMV coat protein in C3H/HeJ mice injected subcutaneously with the multimeric PapMVCP-E2, the monomeric counterpart (PapMVCP27-215-E2), HCV E2 peptide alone, or PBS (ELISA plates were coated with PapMV-CP or PapMV27-215). Data represent the average of antibody titers from 4 mice. These results are representative of two identical and independent experiments. Black arrows on the graphs indicate the booster injections on day 15.

FIG. 15 illustrates the ability of PapMV to strengthen antibody responses to the model antigens (A) hen egg lysozyme (HEL) and (B) ovalbumin (OVA) in BALB/c mice (three per group) immunized on day 0 with antigen alone, antigen plus PapMV, Freund's complete adjuvant (FCA) or LPS from E. coli O111:B4. A representative result from 2 experiments is shown. The antibodies of the serum collected from the immunized animals were isotyped by ELISA on the model antigens (HEL or OVA) for (C) IgG1, (D) IgG 2a and (E) IgG2b.

FIG. 16 presents (A) the amino acid sequences of the C-terminus of the wild-type PapMV coat protein and the recombinant constructs, which comprise a fusion at the C-terminus of the PapMV coat protein of the affinity peptide to OmpC or to OmpF (constructs PapMV OmpC and PapMV OmpF, respectively); (B) SDS-PAGE showing the profile of the purified proteins PapMV, PapMV OmpC, PapMV OmpF, OmpC and OmpF, [First lane: molecular weight markers, second lane; PapMV VLPs, third lane; PapMV OmpC VLPs, fourth lane; PapMV OmpF VLPs, fifth lane; purified OmpC, sixth lane; purified OmpF], and (C) an electron micrograph of the high-speed pellet of the recombinant PapMV OmpC and PapMV OmpF VLPs.

FIG. 17 illustrates high avidity binding of the PapMV VLPs to their respective antigen; (A) presents an ELISA showing the binding the high avidity PapMV OmpC VLPs to the OmpC antigen; (B) presents an ELISA showing the binding the high avidity PapMV OmpF VLPs to the OmpF antigen.

FIG. 18 presents the results of a protection assay against S. typhi challenge in mice, (A) depicts the protective capacity against 100 LD₅₀ of S. typhi in mice immunized with OmpC alone and mice immunized with a preparation containing OmpC+PapMV OmpC VLPs; (B) depicts the protective capacity against 100 LD₅₀ of S. typhi in mice immunized with OmpF alone and mice immunized with a preparation containing OmpF+PapMV OmpF VLPs; (C) depicts the protective capacity against 500 LD₅₀ of S. typhi in mice immunized with OmpC alone and mice immunized with a preparation containing OmpC+PapMV OmpC VLPs, and (D) depicts the protective capacity against 500 LD₅₀ of S. typhi in mice immunized with OmpF alone and mice immunized with a preparation containing OmpF+PapMV OmpF VLPs.

FIG. 19 illustrates (A-D) the evaluation of the antibody response directed to OmpC; the IgG response to OmpC of the isotypes IgG1(A), IgG2a (B), IgG2b (C) and IgG3 (D) was measured between mice immunized with OmpC or a vaccine comprising OmpC+PapMV OmpC VLPs, and (E) that co-immunization of OmpC and PapMV OmpC to mice followed by challenge with S. typhi favours the long lasting active protection of mice against S. typhi infection (as illustrated by % survival) when compared to immunization with OmpC or PapMV OmpC alone.

FIG. 20 illustrates that PapMV virus increased the protective capacity of OmpC porin in mice; (A) Groups of 10 female BALB/c mice were immunized i.p. with 10 μg of OmpC either alone or with 30 μg of PapMV. A booster was given on day 15 using OmpC alone. Control mice were injected with saline (ISS) or PapMV. The challenge of experimental groups was performed on day 21 with a 100 (filled symbols) or a 500 (open symbols) LD₅₀ of S. typhi, and the survival rate was recorded for 10 days after the challenge. Control groups were challenged with 20 LD₅₀ of S. typhi. A representative result of three experiments is shown. (B) Groups of five female BALB/c mice were immunized i.p. on day 0 with 10 μg of OmpC alone or together with 30 μg of PapMV or Freund's incomplete adjuvant (IFA) (1:1, v/v). On day 15, all mice were boosted with 10 μg of OmpC only. Control mice were injected with saline only (isotonic saline solution (ISS)). Antibody titres were measured by enzyme-linked immunosorbent assay (ELISA) on day 21 after the first immunization.

FIG. 21 presents (A) the amino acid sequence (SEQ ID NO:39) of the OmpC precursor from Salmonella enterica subsp. enterica serovar Typhi Ty2 (GenBank Accession No. P0A264); and (B) the amino acid sequence (SEQ ID NO:40) of the OmpF precursor protein from Salmonella enterica subsp. enterica serovar Typhi CT18 (GenBank Accession No. CAD05399).

FIG. 22 presents the amino acid sequence of (A) PapMV coat protein comprising an affinity peptide for binding to OmpC [SEQ ID NO:42], and (B) PapMV coat protein comprising an affinity peptide for binding to OmpF [SEQ ID NO:43]. Differences between the cloned and wild-type sequence are marked in bold and underlined; the affinity peptide sequence is underlined, and the histidine tag is shown in italics.

FIG. 23 presents (A) the amino acid sequences of the C-terminus of the wild-type PapMV coat protein and the recombinant construct that comprises the S. typhi porin loop 6 peptide (shown in bold and underlined) fused between the PapMV coat protein and a 6× His tag (6His) located at the C-terminus of the protein; (B) an SDS-PAGE gel showing the results of purification of recombinant proteins. 1. Molecular weight markers 2. Total bacterial lysates of bacteria prior induction of the expression of the protein PapMV-loop6 3. Total bacterial lysates of bacteria after induction of expression of the protein PapMV-loop6 with IPTG 4. High speed pellet containing VLPs of the purified PapMV-loop6 protein, and (C) an electron micrograph showing PapMV virus, PapMV recombinant VLPs without any fusion and PapMV recombinant VLPs comprising the loop 6 peptide (PapMV-loop6).

FIG. 24 presents (A) the amino acid sequence of the coat protein from the PapMV coat protein comprising the S. typhi porin loop 6 peptide (PapMV-loop6) [SEQ ID NO:46], with the loop 6 peptide sequence underlined, and the histidine tag shown in italics, and (B) the nucleotide sequence encoding the PapMV-loop6 protein [SEQ ID NO:47].

FIG. 25 presents results showing the antibody response toward the PapMV-loop6 construct. Five Balb/C mice were immunised with 100 μg of the PapMV-loop6 s.c. at two-week intervals. The last bleeding was performed on day 28 and the ELISA was performed using: (A) the synthetic and purified loop6 peptide, (B) the PapMV platform and (C) purified OmpC from S. typhi.

FIG. 26 presents the results of a protection assay against S. typhi challenge in mice, (A) depicts the protective capacity of OmpF alone and a preparation containing either OmpF+PapMV OmpF VLPs or OmpF+PapMV against a challenge of 77 LD₅₀ of S. typhi in mice, and (B) depicts the protective capacity of OmpF alone and a preparation containing either OmpF+PapMV OmpF VLPs or OmpF+PapMV against a challenge of 378 LD₅₀ of S. typhi in mice.

FIG. 27 presents (A) the amino acid sequences of the C-terminus of the PapMV SM coat protein and the recombinant construct comprising a fusion of the affinity peptide to OmpC at the C-terminus of the PapMV SM coat protein; (B) SDS-PAGE showing the profile of the purified protein PapMV SM OmpC [First lane: molecular weight markers, second lane; bacterial lysate before induction with IPTG, third lane; bacterial lysation of bacteria expressing high amount of the PapMV SM CP, fourth lane; Purified PapMV SM OmpC protein. PapMV OmpF], and (C) an electron micrograph of the high-speed pellet of the recombinant PapMV SM OmpC at 100,000× magnification.

FIG. 28 presents (A) the amino acid sequence of the PapMV SM coat protein comprising an affinity peptide for binding to OmpC (PapMV SM OmpC) [SEQ ID NO:48], and (B) the nucleotide sequence enconding the PapMV SM OmpC protein [SEQ ID NO:49].

FIG. 29 presents the results of a protection assay against S. typhi challenge in mice, (A) depicts the protective capacity of OmpC alone or a preparation containing either OmpC+PapMV SM OmpC VLPs or OmpC+PapMV VLPs against 105 LD₅₀ of S. typhi in mice, and (B) depicts the protective capacity of OmpC alone or a preparation containing either OmpC+PapMV SM OmpC VLPs or OmpC+PapMV VLPs against 520 LD₅₀ of S. typhi in mice.

FIG. 30 presents the results of an assessment of the stability of S. typhi porins. (A) depicts the SDS-PAGE profile of the purified porins (OmpC+OmpF) (5 μg), lot ISIPOR produced Nov. 9, 1999 (Salazar-Gonzales et al., 2004, Immunology Letters 93), (B) depicts the SDS-PAGE profile of the same ISIPOR lot (0.2 μg; lane 2) in January, 2008, and (C) depicts the SDS-PAGE profiles of purified OmpC protein, lot-1239 purified Apr. 30, 2007 [Left hand panel: Gel run on Apr. 30, 20007; lane 1: molecular weight marker, lane 2: OmpC (10 μg). Right hand panel: Gel run on Jan. 18, 2008, lane 1: OmpC (10 μg)].

FIG. 31 demonstrates that PapMV induces co-stimulatory molecule up-regulation in dendritic cells (DC), macrophages and B cells in vivo. BALB/c mice were immunized intraperitoneally (i.p.) with 30 μg of saline-diluted PapMV (grey line) or with 50 lg of Poly I:C (black line), and control mice were injected with saline (filled histogram). Twenty-four hours after immunization, lymph nodes and spleen cells were collected and stained. Flow cytometry was performed to analyse cell populations defined as: CD11c+ CD11b− (DCs), CD11c− CD11b+ (macrophages) and CD11c− B220+ (B cells). A representative result of two experiments is shown. FITC, fluorescein isothiocyanate; MHC II, major histocompatibility complex class II.

### DETAILED DESCRIPTION OF THE INVENTION

An antigen-presenting system (APS) comprising one or more antigens from an Enterobacteriaceae bacterium in combination with a papaya mosaic virus (PapMV) or a VLP derived from papaya mosaic virus is provided. In accordance with one embodiment of the present invention, the APS is capable of inducing a humoral and/or cellular immune response in an animal. The APS is thus suitable for use as a vaccine against bacterial diseases or conditions, which may require an active participation of one or both of these two branches of the immune system.

In one embodiment of the present invention, the APS comprises one or more Salmonella enterica serovar Typhi (S. typhi) antigens and is suitable for use as a vaccine for the treatment and/or prevention of typhoid fever.

In another embodiment of the present invention, the APS comprises one or more outer membrane protein (Omp) antigens. In a specific embodiment, the APS comprises one or more OmpC and/or OmpF antigens. As is known in the art, regions of these two proteins are conserved across members of the Enterobacteriaceae. Accordingly, in another embodiment, the present invention provides for an APS comprising one or more OmpC and/or OmpF antigens, which is suitable for use as a multivalent vaccine to provide protection against infection by a plurality of enterobacterial species. In a further embodiment, the invention provides for an APS comprising one or more OmpC and/or OmpF antigens, which are suitable for use as a monovalent vaccine.

Definitions

Unless defined otherwise, all technical and scientific terms used herein have the same meaning as commonly understood by one of ordinary skill in the art to which this invention belongs.

As used herein, the term “about” refers to approximately a +/−10% variation from a given value. It is to be understood that such a variation is always included in any given value provided herein, whether or not it is specifically referred to.

The term “adjuvant,” as used herein, refers to an agent that augments, stimulates, actuates, potentiates and/or modulates an immune response in an animal. An adjuvant may or may not have an effect on the immune response in itself.

As used herein, a “chimeric protein” is a protein that is created when two or more genes that normally code for two separate proteins or protein fragments recombine, either naturally or as the result of human intervention, to provide a polynucleotide encoding a protein (the “chimeric protein”) that is a combination of all or part of each of those two proteins. In the context of the present invention, a “fusion protein” is considered to be a “chimeric protein.”

The expression “fusion coat protein” is used herein to refer to a fusion protein in which one of the proteins in the fusion is a PapMV coat protein.

The term “immune response,” as used herein, refers to an alteration in the reactivity of the immune system of an animal in response to an antigen or antigenic material and may involve antibody production, induction of cell-mediated immunity, complement activation, development of immunological tolerance, or a combination thereof.

The terms “effective immunoprotective response” and “immunoprotection,” as used herein, mean an immune response that is directed against one or more antigen so as to protect against disease and/or infection by a pathogen in a vaccinated animal. For purposes of the present invention, protection against disease and/or infection by a pathogen includes not only the absolute prevention of the disease or infection, but also any detectable reduction in the degree or rate of disease or infection, or any detectable reduction in the severity of the disease or any symptom or condition resulting from infection by the pathogen in the vaccinated animal as compared to an unvaccinated infected or diseased animal. An effective immunoprotective response can be induced in animals that were not previously suffering from the disease, have not previously been infected with the pathogen and/or do not have the disease or infection at the time of vaccination. An effective immunoprotective response can also be induced in an animal already suffering from the disease or infected with the pathogen at the time of vaccination. Immunoprotection can be the result of one or more mechanisms, including humoral and/or cellular immunity.

The terms “immune stimulation” and “immunostimulation” as used interchangeably herein, refer to the ability of a molecule, such as a PapMV or PapMV VLP, that is unrelated to an animal pathogen or disease to provide protection to against infection by the pathogen or against the disease by stimulating the immune system and/or improving the capacity of the immune system to respond to the infection or disease. Immunostimulation may have a prophylactic effect, a therapeutic effect, or a combination thereof.

A “recombinant virus” is one in which the genetic material of a naturally-occurring virus has combined with other genetic material.

“Naturally-occurring,” as used herein, as applied to an object, refers to the fact that an object can be found in nature. For example, an organism (including a virus), or a polypeptide or polynucleotide sequence that is present in an organism that can be isolated from a source in nature and which has not been intentionally modified by man in the laboratory is naturally-occurring.

The terms “polypeptide” or “peptide” as used herein is intended to mean a molecule in which there is at least four amino acids linked by peptide bonds.

The expression “viral nucleic acid,” as used herein, may be the genome (or a majority thereof) of a virus, or a nucleic acid molecule complementary in base sequence to that genome. A DNA molecule that is complementary to viral RNA is also considered viral nucleic acid, as is a RNA molecule that is complementary in base sequence to viral DNA.

The term “virus-like particle” (VLP), as used herein, refers to a self-assembling particle which has a similar physical appearance to a virus particle. The VLP may or may not comprise viral nucleic acids. VLPs are generally incapable of replication.

The term “pseudovirus,” as used herein, refers to a VLP that comprises nucleic acid sequences, such as DNA or RNA, including nucleic acids in plasmid form. Pseudoviruses are generally incapable of replication.

The term “vaccine,” as used herein, refers to a material capable of producing an immune response.

The terms “immunogen” and “antigen” as used herein refer to a molecule, molecules, a portion or portions of a molecule, or a combination of molecules, up to and including whole cells and tissues, which are capable of inducing an immune response in a subject alone or in combination with an adjuvant. The immunogen/antigen may comprise a single epitope or may comprise a plurality of epitopes. The term thus encompasses peptides, carbohydrates, proteins, nucleic acids, and various microorganisms, in whole or in part, including viruses, bacteria and parasites. Haptens are also considered to be encompassed by the terms “immunogen” and “antigen” as used herein.

The terms “immunization” and “vaccination” are used interchangeably herein to refer to the administration of a vaccine to a subject for the purposes of raising an immune response and can have a prophylactic effect, a therapeutic effect, or a combination thereof. Immunization can be accomplished using various methods depending on the subject to be treated including, but not limited to, intraperitoneal injection (i.p.), intravenous injection (i.v.), intramuscular injection (i.m.), oral administration, intranasal administration, spray administration and immersion.

The term “prime” and grammatical variations thereof, as used herein, means to stimulate and/or actuate an immune response against an antigen in an animal prior to administering a booster vaccination with the antigen.

As used herein, the terms “treat,” “treated,” or “treating” when used with respect to a disease or pathogen refers to a treatment which increases the resistance of a subject to the disease or to infection with a pathogen (i.e. decreases the likelihood that the subject will contract the disease or become infected with the pathogen) as well as a treatment after the subject has contracted the disease or become infected in order to fight a disease or infection (for example, reduce, eliminate, ameliorate or stabilise a disease or infection).

The term “subject” or “patient” as used herein refers to an animal in need of treatment.

The term “animal,” as used herein, refers to both human and non-human animals, including, but not limited to, mammals, birds and fish, and encompasses domestic, farm, zoo, laboratory and wild animals, such as, for example, cows, pigs, horses, goats, sheep and other hoofed animals, dogs, cats, chickens, ducks, non-human primates, guinea pigs, rabbits, ferrets, rats, hamsters and mice.

The term “substantially identical,” as used herein in relation to a nucleic acid or amino acid sequence indicates that, when optimally aligned, for example using the methods described below, the nucleic acid or amino acid sequence shares at least 70%, at least 75%, at least 80%, at least 85%, at least 90%, at least 95%, at least 96%, at least 97%, at least 98% or at least 99% sequence identity with a defined second nucleic acid or amino acid sequence (or “reference sequence”). “Substantial identity” may be used to refer to various types and lengths of sequence, such as full-length sequence, functional domains, coding and/or regulatory sequences, promoters, and genomic sequences. Percent identity between two amino acid or nucleic acid sequences can be determined in various ways that are within the skill of a worker in the art, for example, using publicly available computer software such as Smith Waterman Alignment (Smith, T. F. and M. S. Waterman (1981) J Mol Biol 147:195-7); “BestFit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool (Altschul, S. F., W. Gish, et al. (1990) J Mol Biol 215: 403-10), and variations thereof including BLAST-2, BLAST-P, BLAST-N, BLAST-X, WU-BLAST-2, ALIGN, ALIGN-2, CLUSTAL, and Megalign (DNASTAR) software. In addition, those skilled in the art can determine appropriate parameters for measuring alignment, including algorithms needed to achieve maximal alignment over the length of the sequences being compared. In general, for amino acid sequences, the length of comparison sequences will be at least 10 amino acids. One skilled in the art will understand that the actual length will depend on the overall length of the sequences being compared and may be at least 20, at least 30, at least 40, at least 50, at least 60, at least 70, at least 80, at least 90, at least 100, at least 110, at least 120, at least 130, at least 140, at least 150, or at least 200 amino acids, or it may be the full-length of the amino acid sequence. For nucleic acids, the length of comparison sequences will generally be at least 25 nucleotides, but may be at least 50, at least 100, at least 125, at least 150, at least 200, at least 250, at least 300, at least 350, at least 400, at least 450, at least 500, at least 550, or at least 600 nucleotides, or it may be the full-length of the nucleic acid sequence.

The terms “corresponding to” or “corresponds to” indicate that a nucleic acid sequence is identical to all or a portion of a reference nucleic acid sequence. In contradistinction, the term “complementary to” is used herein to indicate that the nucleic acid sequence is identical to all or a portion of the complementary strand of a reference nucleic acid sequence. For illustration, the nucleic acid sequence “TATAC” corresponds to a reference sequence “TATAC” and is complementary to a reference sequence “GTATA.”

Antigen-Presenting System (APS)

An antigen-presenting system (APS) of the present invention comprises one or more enterobacterial antigens in combination with a papaya mosaic virus (PapMV) or a VLP derived from a PapMV coat protein. By “derived from” it is meant that the VLP comprises coat proteins that have an amino acid sequence substantially identical to the sequence of the wild-type coat protein and may optionally include one or more antigens attached to the coat protein, as described in more detail below. The PapMV coat protein can be the wild-type coat protein or a modified version thereof which is capable of multimerization and self-assembly to form a VLP. In one embodiment of the present invention, as described in detail below, the APS comprises one or more antigens from the enterobacterium Salmonella enterica Serovar Typhi (S. typhi). In another embodiment, the APS includes one or more antigens from a different bacterium of the family Enterobacteriaceae.

The one or more antigens comprised by the APS can be conjugated to a coat protein of the PapMV or PapMV VLP, or they may be non-conjugated (i.e. separate from the PapMV or PapMV VLP), or the APS may include both conjugated and non-conjugated antigens. In this latter context, the non-conjugated antigens are referred to as additional isolated antigens (AIAs). The AIAs may be the same as or different than the conjugated antigen(s). Conjugation can be, for example, by genetic fusion with the coat protein, or binding via covalent, non-covalent or affinity means.

The PapMV or PapMV VLP included in the APS thus acts as an immunopotentiator capable of potentiating an immune response in an animal, and optionally as a carrier for the one or more antigens comprised by the APS. In accordance with one embodiment of the present invention, the APS is capable of inducing a humoral and/or cellular immune response in an animal and thus is suitable for use as a vaccine, for example, against an enterobacterial infection.

Papaya Mosaic Virus (PapMV) and PapMV VLPs

The APS of the present invention comprises either PapMV or PapMV VLPs. PapMV VLPs are formed from recombinant PapMV coat proteins that have multimerised and self-assembled to form a VLP. When assembled, each VLP comprises a long helical array of coat protein subunits. The wild-type virus comprises over 1200 coat protein subunits and is about 500 nm in length. PapMV VLPs that are either shorter or longer than the wild-type virus can still, however, be effective. In one embodiment of the present invention, the VLP comprises at least 40 coat protein subunits. In another embodiment, the VLP comprises between about 40 and about 1600 coat protein subunits. In an alternative embodiment, the VLP is at least 40 nm in length. In another embodiment, the VLP is between about 40 nm and about 600 nm in length.

The VLPs of the present invention can be prepared from a plurality of recombinant coat proteins having identical amino acid sequences, such that the final VLP when assembled comprises identical coat protein subunits, or the VLP can be prepared from a plurality of recombinant coat proteins having different amino acid sequences, such that the final VLP when assembled comprises variations in its coat protein subunits.

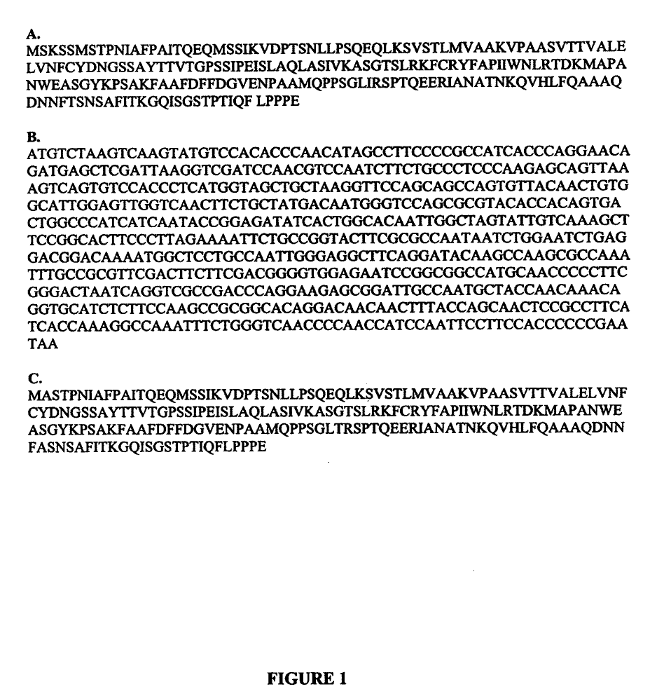

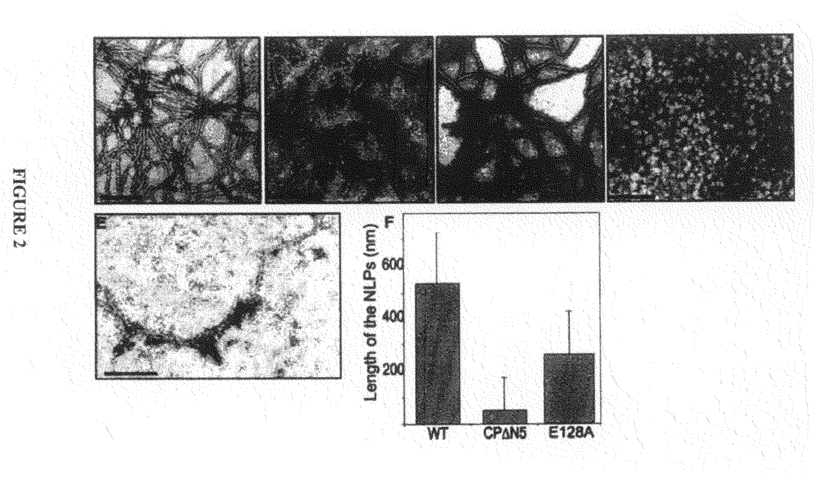

The coat protein used to form the VLP can be the entire PapMV coat protein, or part thereof, or it can be a genetically modified version of the PapMV coat protein, for example, comprising one or more amino acid deletions, insertions, replacements and the like, provided that the coat protein retains the ability to multimerise and assemble into a VLP. The amino acid sequence of the wild-type PapMV coat (or capsid) protein is known in the art (see, Sit, et al., 1989, J. Gen. Virol., 70:2325-2331, and GenBank Accession No. NP_{—}044334.1) and is provided herein as SEQ ID NO:1 (see FIG. 1A). The nucleotide sequence of the PapMV coat protein is also known in the art (see, Sit, et al., ibid., and GenBank Accession No. NC_{—}001748 (nucleotides 5889-6536)) and is provided herein as SEQ ID NO:2 (see FIG. 1B).

As noted above, the amino acid sequence of the recombinant PapMV coat protein comprised by the VLP need not correspond precisely to the parental sequence, i.e. it may be a modified or “variant sequence.” For example, the recombinant protein may be mutagenized by substitution, insertion or deletion of one or more amino acid residues so that the residue at that site does not correspond to the parental (reference) sequence. One skilled in the art will appreciate, however, that such mutations will not be extensive and will not dramatically affect the ability of the recombinant coat protein to multimerise and assemble into a VLP. The ability of a variant version of the PapMV coat protein to assemble into multimers and VLPs can be assessed, for example, by electron microscopy following standard techniques, such as the exemplary methods set out in the Examples provided herein.

Recombinant coat proteins that are fragments of the wild-type protein that retain the ability to multimerise and assemble into a VLP (i.e. are “functional” fragments) are, therefore, also contemplated by the present invention. For example, a fragment may comprise a deletion of one or more amino acids from the N-terminus, the C-terminus, or the interior of the protein, or a combination thereof. In general, functional fragments are at least 100 amino acids in length. In one embodiment of the present invention, functional fragments are at least 150 amino acids, at least 160 amino acids, at least 170 amino acids, at least 180 amino acids, and at least 190 amino acids in length. Deletions made at the N-terminus of the protein should generally delete fewer than 25 amino acids in order to retain the ability of the protein to multimerise.

In accordance with the present invention, when a recombinant coat protein comprises a variant sequence, the variant sequence is at least about 70% identical to the reference sequence. In one embodiment, the variant sequence is at least about 75% identical to the reference sequence. In other embodiments, the variant sequence is at least about 80%, at least about 85%, at least about 90%, at least about 95%, and at least about 97% identical to the reference sequence. In a specific embodiment, the reference amino acid sequence is SEQ ID NO:1.

In one embodiment of the present invention, the VLP comprises a genetically modified (i.e. variant) version of the PapMV coat protein. In another embodiment, the PapMV coat protein has been genetically modified to delete amino acids from the N- or C-terminus of the protein and/or to include one or more amino acid substitutions. In a further embodiment, the PapMV coat protein has been genetically modified to delete between about 1 and about 10 amino acids from the N- or C-terminus of the protein.

In a specific embodiment, the PapMV coat protein has been genetically modified to remove one of the two methionine codons that occur proximal to the N-terminus of the protein (i.e. at positions 1 and 6 of SEQ ID NO:1) and can initiate translation. Removal of one of the translation initiation codons allows a homogeneous population of proteins to be produced. The selected methionine codon can be removed, for example, by substituting one or more of the nucleotides that make up the codon such that the codon codes for an amino acid other than methionine, or becomes a nonsense codon. Alternatively all or part of the codon, or the 5′ region of the nucleic acid encoding the protein that includes the selected codon, can be deleted. In a specific embodiment of the present invention, the PapMV coat protein has been genetically modified to delete between 1 and 5 amino acids from the N-terminus of the protein. In a further embodiment, the genetically modified PapMV coat protein has an amino acid sequence substantially identical to SEQ ID NO:3.

When the recombinant coat protein comprises a variant sequence that contains one or more amino acid substitutions, these can be “conservative” substitutions or “non-conservative” substitutions. A conservative substitution involves the replacement of one amino acid residue by another residue having similar side chain properties. As is known in the art, the twenty naturally occurring amino acids can be grouped according to the physicochemical properties of their side chains. Suitable groupings include alanine, valine, leucine, isoleucine, proline, methionine, phenylalanine and tryptophan (hydrophobic side chains); glycine, serine, threonine, cysteine, tyrosine, asparagine, and glutamine (polar, uncharged side chains); aspartic acid and glutamic acid (acidic side chains) and lysine, arginine and histidine (basic side chains). Another grouping of amino acids is phenylalanine, tryptophan, and tyrosine (aromatic side chains). A conservative substitution involves the substitution of an amino acid with another amino acid from the same group. A non-conservative substitution involves the replacement of one amino acid residue by another residue having different side chain properties, for example, replacement of an acidic residue with a neutral or basic residue, replacement of a neutral residue with an acidic or basic residue, replacement of a hydrophobic residue with a hydrophilic residue, and the like.

In one embodiment of the present invention, the variant sequence comprises one or more non-conservative substitutions. Replacement of one amino acid with another having different properties may improve the properties of the coat protein. For example, as described herein, mutation of residue 128 of the coat protein improves assembly of the protein into VLPs. In one embodiment of the present invention, therefore, the coat protein comprises a mutation at residue 128 of the coat protein in which the glutamic residue at this position is substituted with a neutral residue. In a further embodiment, the glutamic residue at position 128 is substituted with an alanine residue.

Likewise, the nucleic acid sequence encoding the recombinant coat protein need not correspond precisely to the parental reference sequence but may vary by virtue of the degeneracy of the genetic code and/or such that it encodes a variant amino acid sequence as described above. In one embodiment of the present invention, therefore, the nucleic acid sequence encoding the recombinant coat protein is at least about 70% identical to the reference sequence. In another embodiment, the nucleic acid sequence encoding the recombinant coat protein is at least about 75% identical to the reference sequence. In other embodiments, the nucleic acid sequence encoding the recombinant coat protein is at least about 80%, at least about 85% or at least about 90% identical to the reference sequence. In a specific embodiment, the reference nucleic acid sequence is SEQ ID NO:2.

The PapMV VLP coat protein may optionally be genetically fused to one or more antigens, an affinity peptide or other short peptide sequence to facilitate attachment of one or more antigens, as described in more detail below.

Antigens

The APS of the present invention comprises one or more antigens from gram-negative bacteria of the family Enterobacteriaceae. Examples of suitable enterobacteria include, but are not limited to, species of the genus Escherichia, Salmonella, Yersinia, Shigella, Proteus, Klebsiella and Enterobacter. The antigen may be an immunogen, epitope, mimotope or antigenic determinant from an enterobacterium and may be, for example, a peptide, a protein, a nucleic acid or a superficial bacterial antigenic component (SBAC), such as a lipopolysaccharide (LPS), capsular antigen (proteinacious or polysaccharide in nature), or a flagellar component, or it may be an inactivated or attenuated version of the bacterium.

Candidate antigens can be selected based on the desired end use of the APS, for example which species of bacteria it is directed against, whether it is intended for use as a multivalent or monovalent vaccine and/or the animal to which it is to be administered. Appropriate candidate antigens can readily be selected by the skilled worker. A number of antigenic enterobacterial components are known in the art and are suitable candidate antigens for inclusion in the APS of the present invention. It will also be understood by the skilled worker that suitable candidate antigen(s) may be selected for incorporation into an APS of the invention based on testing for their ability to induce an immune response in an animal using standard immunological techniques known in the art.

In one embodiment of the present invention, candidate antigens are selected from a species of the genus Escherichia, Salmonella, Yersinia, Shigella, Proteus, Klebsiella or Enterobacter. In another embodiment of the present invention, candidate antigens are selected from a species of the genus Escherichia, Salmonella, Yersinia, Shigella, Klebsiella or Enterobacter. In a further embodiment, candidate antigens are selected from a species of the genus Escherichia or Salmonella. In another embodiment, candidate antigens are selected from a species of the genus Salmonella In a specific embodiment, the candidate antigens are S. typhi antigens.

Examples of suitable candidate superficial bacterial antigenic components (SBACs) include various enterobacterial H (flagellar component) antigens and O (LPS) antigens, the S. typhi Vi (capsular polysaccharide) antigen, the E. coli K and CFA (capsular component) antigens and the E. coli fimbrial adhesin antigens (K88 and K99). Examples of suitable candidate antigenic proteins include the outer membrane proteins (Omps), also known as porins (Secundino et al, 2006, Immunology 117:59); related porins such as the S. typhi iron-regulated outer membrane protein (IROMP, Sood et al., 2005, Mol Cell Biochem 273:69-78), and heat shock proteins (HSPs) including, but not limited to S. typhi HSP40 (Sagi et al., 2006, Vaccine 24:7135-7141). Non-limiting examples of suitable porins include OmpC and OmpF, which are found in numerous Salmonella and Escherichia species. Orthologues of OmpC and OmpF are also found in other Enterobacteriaceae and are suitable candidate antigenic proteins for the purposes of the present invention. In addition, Omp1B (Shigella flexneri), OmpC2 (Yersinia pestis), OmpD (S. enterica), OmpK36 (Klebsiella pneumonie), OmpN (E. coli) and OmpS (S. enterica) are suitable candidates, based on conserved regions of sequences found in the porin proteins of the Enterobacteriaceae family (Diaz-Quinonez et al., 2004, Infect. and Immunity 72:3059-3062).

Also suitable as candidate antigens are metal transporter proteins, such as the E. coli SitABCD, MntH and FeoB metal transporters (Sabri, et al, 2008, Infect. Immun. 76:601-611; Epub ahead of print Nov. 19, 2007).

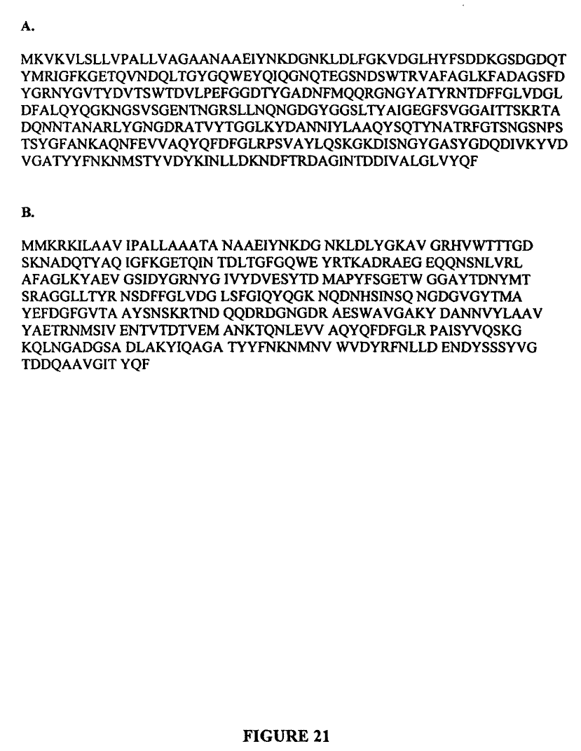

The sequences of antigenic proteins from various enterobacteria are known in the art and are readily accessible from GenBank database maintained by the National Center for Biotechnology Information (NCBI). For example; GenBank Accession No. P0A264 (SEQ ID NO:39; also shown in FIG. 21A) and GenBank Accession No. NP_{—}804453: OmpC (S. enterica subsp. enterica serovar Typhi Ty2); GenBank Accession No. CAD05399 (SEQ ID NO:40; also shown in FIG. 21B): OmpF precursor protein (S. enterica subsp. enterica serovar Typhi CT18); GenBank Accession No. 16761195: OmpC (S. enterica serovar Typhimurium); GenBank Accession No. 47797: OmpC (S. enterica serovar Typhi); GenBank Accession No. 8953564: OmpC (S. enterica serovar Minnesota); GenBank Accession No. 19743624: OmpC (S. enterica serovar Dublin); GenBank Accession No. 19743622: OmpC (S. enterica serovar Gallinarum); GenBank Accession No. 26248604: OmpC (E. coli); GenBank Accession No. 24113600: Omp1B (Shigella flexneri); GenBank Accession No. 16764875: OmpC2 (Yersinia pestis); GenBank Accession No. 16764916: OmpD (S. enterica Serovar Typhimurium); GenBank Accession No. 151149831: OmpK36 (Klebsiella pneumonie); GenBank Accession No. 3273514: OmpN (E. coil), and GenBank Accession No. 16760442: OmpS (S. enterica serovar Typhi).

In one embodiment of the present invention, the antigen(s) included in the APS are protein antigens. A protein antigen can be a full-length protein, a substantially full-length protein (for example, a protein comprising a N-terminal and/or C-terminal deletion of about 25 amino acids or less), an antigenic fragment of the protein, or a combination thereof. The full-length protein can be, when applicable, a precursor form of the protein or the mature (processed) form of the protein. An antigenic fragment can comprise one, or a plurality of epitopes, and thus may range in size from a peptide of a few amino acids (for example, at least 4 amino acids) to a polypeptide several hundred amino acids in length. In one embodiment of the present invention, an antigenic fragment is between about 4 amino acids and about 250 amino acids in length. In another embodiment, the antigenic fragment is between about 5 amino acids and about 200 amino acids in length. In other embodiments, the antigenic fragment is between about 5 amino acids and about 150 amino acids in length, between about 5 amino acids and about 100 amino acids in length, between about 5 amino acids and about 75 amino acids in length, between about 5 amino acids and about 70 amino acids in length, between about 5 amino acids and about 60 amino acids in length, and between about 5 amino acids and about 50 amino acids in length.

In one embodiment of the present invention, the antigens incorporated into the APS are full-length or substantially full-length proteins. In another embodiment, the antigens incorporated into the APS are full-length or substantially full-length porin proteins. In a further embodiment, the antigens incorporated into the APS are full-length or substantially full-length OmpC and/or OmpF proteins, or full-length or substantially full-length OmpC and/or OmpF orthologues.

As shown in Table 1, the sequences of the S. typhi OmpC protein and OmpC orthologues from other enterobacteria are highly conserved. The sequences are even more highly conserved between Salmonella spp. (see Table 1). Likewise, the sequences of the S. typhi OmpF protein and OmpF orthologues from other Salmonella species are highly conserved (see Table 2). Accordingly, in one embodiment of the present invention, at least one antigen incorporated into the APS is a full-length or substantially full-length OmpC or OmpC orthologue that has an amino acid sequence that is at least about 75% identical to the sequence of the S. typhi OmpC protein as shown in FIG. 21A [SEQ ID NO:39]. In another embodiment, at least one antigen incorporated into the APS is a full-length or substantially full-length OmpC or OmpC orthologue that has an amino acid sequence that is at least about 78% identical to the sequence of the S. typhi OmpC protein as shown in FIG. 21A [SEQ ID NO:39]. In a further embodiment of the present invention, at least one antigen incorporated into the APS is a full-length or substantially full-length OmpC or OmpC orthologue that has an amino acid sequence that is at least about 80% identical to the sequence of the S. typhi OmpC protein as shown in FIG. 21A [SEQ ID NO:39].

In another embodiment of the present invention, at least one antigen incorporated into the APS is a full-length or substantially full-length OmpF or OmpF orthologue that has an amino acid sequence that is at least about 75% identical to the sequence of the S. typhi OmpF protein as shown in FIG. 21B [SEQ ID NO:40]. In a further embodiment of the present invention, at least one antigen incorporated into the APS is a full-length or substantially full-length OmpF or OmpF orthologue that has an amino acid sequence that is at least about 78% identical to the sequence of the S. typhi OmpF protein as shown in FIG. 21B [SEQ ID NO:40]. In another embodiment of the present invention, at least one antigen incorporated into the APS is a full-length or substantially full-length OmpF or OmpF orthologue that has an amino acid sequence that is at least about 80% identical to the sequence of the S. typhi OmpF protein as shown in FIG. 21B [SEQ ID NO:40].

In a specific embodiment of the present invention, the antigens incorporated into the APS are full-length or substantially full-length S. typhi OmpC and/or OmpF proteins.

TABLE 1
Sequence Identity of OmpC and OmpC Orthologues from Various
Enterobacteria

| Organism | Protein | Reference | % Identity¹ |
|---|---|---|---|
| Salmonella typhimurium | OmpC | P0A263 | 100 |
| LT2 | | | |
| Salmonella bongori | ORF_2828 (Putative | coliBase²: GL026809³ | 99 |
| | OmpC) | | |
| Salmonella enteritidis | ORF_1402 (Putative | coliBase²: GL063386³ | 98 |
| PT4 | OmpC) | | |
| Salmonella gallinarum | ORF_222 (Putative | coliBase²: GL064166³ | 98 |
| 287/91 | OmpC) | | |
| Escherichia coli | OmpC | Q8XE41 | 80 |
| O157:H7 EDL933 | | | |
| Shigella dysenteriae | ORF_14 (Putative | coliBase²: GL018139³ | 78 |
| M131649 (M131)] | OmpC) | | |
| Shigella flexneri 2a | Omp1b | Q83QU7 | 78 |
| 2457T | | | |

¹% identity is relative to the S. typhi OmpC protein (GenBank Accession No. P0A264) and was determined using the BLASTP 2.2.3 [Apr-24-2002] program (Altschul, S. F., et al., (1997), Nucleic Acids Res. 25: 3389-3402.
²Nucleic Acids Research, 2004, Vol. 32, Database issue D296-D299.
³GL numbers as of Jan. 23, 2007.

TABLE 2
Sequence Identity of OmpF and OmpF Orthologues from Various
Enterobacteria

| | | | % |
|---|---|---|---|
| Organism | Protein | Reference | Identity¹ |
| Salmonella | ORF_34 | coliBase²: GL060731³ | 100 |
| enteritidis PT4 | | | |
| Salmonella | ORF_21 | coliBase²: GL069216³ | 99 |
| gallinarum 287/91 | | | |
| Salmonella | ORF_287 | coliBase²: GL0044362³ | 99 |
| typhimurium DT104 | | | |
| Salmonella bongori | ORF_1160 | coliBase²: GL025398³ | 98 |
| Escherichia coli | ORF_2 | coliBase²: GL037694³ | 58 |
| DH10B | | | |
| Shigella flexneri 2a | OmpF | Q83RY7 | 58 |
| 2457T | | | |

¹% identity is relative to the S. typhi OmpF protein (GenBank Accession No. CAD05399) and was determined using the BLASTP 2.2.3 [Apr-24-2002] program (Altschul, S. F., et al., (1997), Nucleic Acids Res. 25: 3389-3402.
²Nucleic Acids Research, 2004, Vol. 32, Database issue D296-D299.
³GL numbers as of Jan. 23, 2007.

In an alternative embodiment, the antigens incorporated into the APS are antigenic fragments of a protein. Various antigenic regions of the above-noted proteins have been identified and are suitable candidate antigens for use in the APS of the present invention. For example, surface exposed loops of outer membrane proteins (Omps) constitute suitable candidate antigenic fragments for incorporation into the APS of the present invention. Loop 6 (L6) and loop (L7) peptides of OmpC isolated from S. typhi are non-limiting examples of such. The sequences of L6 and L7 peptides of S. typhi OmpC have been reported and identified as: GTSNGSNPST (SEQ ID NO:4) and KDISNGYGASYGDQ (SEQ ID NO:5), respectively (Paniagua-Solis et al., 1996, FEMS 141:31-36). Antigenic fragments comprising all or a part of the sequences as set forth in SEQ ID NO:4 and SEQ ID NO:5 are also suitable candidates. For example, the porin fragment having the sequence GTSNGSNPSTSYGFAN [SEQ ID NO:50], which comprises the key amino acids of the L6 peptide, and the porin fragment having the sequence QSKGKDISNGYGASYGDQD [SEQ ID NO:54], which comprises the key amino acids of the L7 peptide.

Other antigenic epitopes are also known. For example, specific fragments of OmpC other than the L6 and L7 peptides can be employed as candidate antigens for inclusion in the APS of the present invention, including the OmpC-73 peptide [SEQ ID NO:6] and OmpC-132 peptide [SEQ ID NO:7] (see Table 3) (Diaz-Quinonez et al., 2004, Infect. and Immunity 72:3059-3062). The sequences of these peptides are highly conserved across different bacteria of the Enterobacteriaceae family making these peptides suitable as candidate antigens for inclusion in an APS intended for multivalent vaccine. Examples of other epitope sequences are shown in Table 3 as SEQ ID NOs: 8, 9 and 10. Variable regions of the S. typhi OmpC protein have also been identified as potential B-cell epitopes that are unique to S. typhi (Arockiasamy and Krishnaswamy, 1995, J. Biosci, 20:235-243) (see Table 3, SEQ ID NOs:11, 12, 13, 14, 15, 16, 17 and 18). Antigenic fragments comprising these regions are also candidate antigens for inclusion in the APS of the present invention.

TABLE 3

Sequences of Canadidate Epitopes from OmpC and Other
Enterobacterial Porins

| | | | |
|---|---|---|---|
| | | | SEQ |
| Protein | Bacterial strain | Sequence of epitope | ID NO |

| | | | |
|---|---|---|---|
| OmpC | S. enterica serovar | TRVAFAGL | 6 |
| | Typhimurium; serovar | | |
| | Typhi; serovar | | |
| | Minnesota; serovar | | |
| | Dublin; serovar | | |
| | Gallinarum and | | |
| | Escherichia coli | | |
| Omp1b | Shigella flexneri | TRVAFAGL | 6 |
| OmpS | S. enterica serovar Typhi | TRLAFAGL | 9 |
| OmpN | Escherichia coli | TRLAFAGL | 9 |
| OmpD | S. enterica serovar | TRLAFAGL | 9 |
| | Typhimurium | | |
| OmpC2 | Yersinia pestis | TRLGFAGL | 10 |
| OmpK36 | Klebsiella pneumoniae | TRLAFAGL | 9 |
| OmpC | S. enterica serovar | RNTDFFGL | 7 |
| | Typhimurium; serovar | | |
| | Typhi; serovar | | |
| | Minnesota; serovar | | |
| | Dublin; serovar | | |
| | Gallinarum and | | |
| | Escherichia coli | | |
| Omp1b | Shigella flexneri | RNSDFFGL | 8 |
| OmpS | S. enterica serovar Typhi | RNTDFFGL | 7 |
| OmpN | Escherichia coli | RNTDFFGL | 7 |
| OmpD | S. enterica serovar | RNTDFFGL | 7 |
| | Typhimurium | | |
| OmpC2 | Yersinia pestis | RNTDFFGL | 7 |
| OmpK36 | Klebsiella pneumoniae | RNSDFFGL | 8 |
| OmpC (amino | S. enterica serovar Typhi | SDDKGSD | 11 |
| acids 45-51) | | | |
| OmpC (amino | S. enterica serovar Typhi | NQTEGSNDS | 12 |
| acids 84-92) | | | |
| OmpC (amino | S. enterica serovar Typhi | NYGVTYDVTSWTDVLPE | 13 |
| acids 114-147) | | FGGDTYGADNFMQQRGN | |
| OmpC (amino | S. enterica serovar Typhi | GSVSGENTNGRSLLNQ | 14 |
| acids 175-190) | | | |
| OmpC (amino | S. enterica serovar Typhi | RTADQNNTANARLYGNG | 15 |
| acids 218-236) | | DR | |
| OmpC (amino | S. enterica serovar Typhi | NATRFGTSNGSNPSTSYG | 16 |
| acids 262-282) | | FAN | |
| OmpC (amino | S. enterica serovar Typhi | DISNGYGASYGDQDIVK | 17 |
| acids 312-328) | | | |
| OmpC (amino | S. enterica serovar Typhi | NLLDKNDETRDAGINTD | 18 |
| acids 351-368) | | | |

In one embodiment of the present invention, the APS comprises a full-length Omp or a fragment that encompasses several of the above-noted epitopes. In another embodiment, the antigen comprises a sequence that is conserved across variants, for example a conserved region defined by amino acids TRXXFAGL (SEQ ID NO:19, where X can be V, L or G), or amino acids RNXDFFGL (SEQ ID NO:20, where X can be T or S).

In one embodiment of the present invention, at least one antigen selected for incorporation in the APS is a porin protein antigen. In accordance with another embodiment, at least one antigen is a whole porin protein. In accordance with yet another embodiment, candidate antigens for inclusion in the APS are OmpC and/or OmpF antigens. In a further embodiment, candidate antigens for inclusion in the APS are peptides of OmpC or OmpF, for example, a loop 6, loop 7, OmpC-73 or OmpC-132 peptide, or variant thereof. In another embodiment, the candidate antigens comprise an amino acid sequence substantially identical to the sequence as set forth in SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19 or SEQ ID NO:20, or a fragment thereof. In a further embodiment, the candidate antigens comprise an amino acid substantially identical to the sequence as set forth in SEQ ID NO:4 or SEQ ID NO:5, or a fragment thereof.

As noted above, the antigen(s) selected can vary in size and the size of antigen selected is not critical to the practice of the present invention. The antigen may be specific or recognised by surface structures on T cells, B cells, NK cells and macrophages, or Class I or Class II APC associated cell surface structures. In one embodiment, the invention is especially useful for small weakly immunogenic antigens. The selected antigen(s) should not interfere with the assembly of a PapMV or PapMV VLP, or with the ability of a PapMV or PapMV VLP to immunopotentiate an immune response. Suitable antigens from the candidate antigens described above can be readily selected by the skilled worker using standard techniques such as those described in the sections below and in the Examples.

The APS of the present invention may comprise one or more antigens each having a single epitope capable of triggering a specific immune response, or the APS may comprise one or more antigen, wherein each antigen comprises a plurality of epitopes. The antigens comprised by the APS can be the same, or they can be different, and when a plurality of protein antigens are present, they may be derived from a single protein or from a plurality of proteins, including antigens derived more than one type of protein (for example, antigens derived from an Omp and an HSP). APSs comprising antigens representing a combination of enterobacterial antigenic proteins (for example, antigens derived from more than one enterobacterium) are also contemplated, as are APSs that comprise antigens from the same conserved region of a protein but which represent variations of the protein seen in different enterobacteria.

In a specific embodiment of the present invention, the APS comprises one or more antigens that are conserved across different members of the Enterobacteriaceae, or a sub-group of the Enterobacteriaceae. For example, OmpC or fragments thereof, and/or OmpF, or fragments thereof. Such antigens are suitable for inclusion in an APS intended for use as a multivalent vaccine. Sequence comparisons indicate a high degree of conservation among OmpC/OmpF and their enterobacterial orthologues including stretches of identical amino acids. In fact, among all Salmonella spp., OmpC and OmpF demonstrate 98-100% identity (see Table 1). Comparison of these proteins with orthologues from other enterobacteria including E. coli, Shigella spp., Klebsiella pneumoniae, and Enterobacter aerogenes demonstrate 75%-85% identity with the protein sequences from Salmonella enterica serovar typhi (see Table 2).

The present invention further contemplates that the APS can comprise an antigen that is an entire protein or fragment thereof in combination with one or more superficial bacterial antigenic components (SBACs) (for example, an O antigen). Thus, in accordance with one embodiment, the APS may comprise a combination of one or more SBACs and one or more antigenic proteins, peptides or fragments thereof.

As described above, an APS of the present invention can comprise one or more conjugated antigens and one or more additional isolated antigens (AIAs). The AIAs can be the same as, or they may differ from, the conjugated antigens of the APS. Various combinations of conjugated antigens and AIAs are contemplated and include the following non-limiting examples: one or more conjugated antigens from a first enterobacterial species and one or more AIAs from the same enterobacterial bacterial species; one or more conjugated antigens from a first enterobacterial species and one or more AIAs from another enterobacterial species; one or more conjugated protein or protein fragment antigens and one or more whole protein or protein fragment AIAs; one or more conjugated SBAC antigens and one or more SBAC AIAs; one or more conjugated protein or protein fragment antigens and one or more SBAC AIAs (or vice versa); one or more conjugated whole protein antigens and one or more whole protein AIM; one or more conjugated whole protein antigens and one or more AIAs that are peptides of said whole protein. The one or more conjugated antigens and the AIAs can belong to the same family of proteins (for example, Omps) or they can belong to different families (for example, Omps and HSPs).

In one embodiment of the present invention, the APS comprises one or more conjugated antigens and one or more AIAs that are whole proteins. In accordance with another embodiment, at least one AIA is a whole porin protein. In accordance with another embodiment, at least one MA is OmpC or OmpF. In a further embodiment, at least one AIA is a peptide of OmpC, for example, a loop 6, loop 7, OMPC-73 or OMPC-132 peptide or variant thereof, or a peptide of OmpF. In another embodiment, at least one AIA comprises an amino acid sequence substantially identical to the sequence as set forth in SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID SEQ ID NO:16, SEQ ID NO:17, SEQ ID NO:18, SEQ ID NO:19 or SEQ ID NO:20. In a further embodiment, the conjugated antigen(s) and the AIA(s) comprised by the APS are the same.

Antigen PapMV and Antigen PapMV-VLP Combinations

As noted above, the one or more antigens comprised by the APS can be conjugated to a coat protein of the PapMV of PapMV VLP, or they may be present in the APS in a non-conjugated form (i.e. simply combined with the PapMV or PapMV VLP), or they may be present in both conjugated and non-conjugated form. Conjugation can be, for example, by genetic fusion with the coat protein, or binding via covalent, non-covalent or affinity means. Combination of the antigen(s) with the PapMV or VLP, however, should not interfere with the recognition of the antigen by the host's immune system or the ability of the PapMV or VLP to potentiate the immune response.

In accordance one embodiment of the present invention, the one or more antigens are conjugated to a coat protein of a PapMV VLP. As the VLP comprises multiple copies of self-assembled coat protein, attaching the antigen to the coat protein allows presentation of the antigens in an organized fashion on the surface of the VLP. In accordance with this embodiment, the VLP comprises coat proteins that include a first portion that is a recombinant PapMV coat protein conjugated to a second portion that comprises one or more antigens. As the antigen-conjugated coat protein retains the ability to assemble with other fusion coat proteins or with wild-type coat protein to form an immunogen-carrier VLP, antigens are selected for conjugation that do not affect this ability.

In order to allow presentation of the antigen on the surface of the VLP and enhance immune recognition of the antigen, the antigen is preferably attached to a region of the coat protein that is disposed on the outer surface of the VLP. Thus the antigen can be inserted near, or attached at, the amino- (N-) or carboxy- (C-) terminus of the coat protein, or it can be inserted into, or attached to, an internal loop of the coat protein which is disposed on the outer surface of the VLP. An example of such a loop would be the region comprised by amino acids 49 to 52 of the PapMV coat protein as set forth on SEQ ID NO:1. In one embodiment of the present invention, the antigen is present at the C-terminus of the PapMV coat protein.

In accordance with one embodiment of the invention, the APS comprises a PapMV coat protein genetically fused to one or more antigens. In order to avoid the possibility of the antigen interfering with the ability of the PapMV coat protein-antigen fusion to self-assemble into a VLP, antigens selected for genetic fusion to the PapMV coat protein are typically less than about 50 amino acids in length. In one embodiment of the present invention, the antigens selected for genetic fusion to the PapMV coat protein are typically less than about 45 amino acids in length.

Larger antigens can be readily incorporated into the APS by simple combination with the PapMV or VLP, or by chemical cross-linking or affinity attachment, as described in more detail below. These latter methods of attachment can facilitate the coupling of an antigen to the PapMV and lead to an improved immune response to the antigen when administered to an animal.

In another embodiment, the antigen(s) are chemically cross-linked to the coat protein, for example, by covalent or non-covalent (such as, ionic, hydrophobic, hydrogen bonding, or the like) attachment. The antigen and/or coat protein can be modified to facilitate such cross-linking as is known in the art, for example, by addition of a functional group or chemical moiety to the protein and/or antigen, for example at the C- or N-terminus or at an internal position. Exemplary modifications include the addition of functional groups such as S-acetylmercaptosuccinic anhydride (SAMSA) or S-acetyl thioacetate (SATA), or addition of one or more cysteine residues. Other cross-linking reagents are known in the art and many are commercially available (see, for example, catalogues from Pierce Chemical Co. and Sigma-Aldrich). Examples include, but are not limited to, diamines, such as 1,6-diaminohexane, 1,3-diamino propane and 1,3-diamino ethane; dialdehydes, such as glutaraldehyde; succinimide esters, such as ethylene glycol-bis(succinic acid N-hydroxysuccinimide ester), disuccinimidyl glutarate, disuccinimidyl suberate, N-(g-Maleimidobutyryloxy) sulfosuccinimide ester and ethylene glycol-bis(succinimidylsuccinate); diisocyantes, such as hexamethylenediisocyanate; bis oxiranes, such as 1,4 butanediyl diglycidyl ether; dicarboxylic acids, such as succinyidisalicylate; 3-maleimidopropionic acid N-hydroxysuccinimide ester, and the like. Many of the above-noted cross-linking agents incorporate a spacer that distances the antigen from the VLP. The use of other spacers is also contemplated by the invention. Various spacers are known in the art and include, but are not limited to, 6-aminohexanoic acid; 1,3-diamino propane; 1,3-diamino ethane; and short amino acid sequences, such as polyglycine sequences, of 1 to 5 amino acids.

To facilitate covalent attachment of the one or more antigen to the coat protein of the VLP, the coat protein can be genetically fused to a short peptide or amino acid linker that is exposed in the surface of the VLP and provides an appropriate site for chemical attachment of the antigen. For example, short peptides comprising cysteine residues, or other amino acid residues having side chains that are capable of forming covalent bonds (for example, acidic and basic residues) or that can be readily modified to form covalent bonds as known in the art. The amino acid linker or peptide can be, for example, between one and about 20 amino acids in length. In one embodiment, the coat protein is fused with a short peptide comprising one or more lysine residues, which can be covalently coupled, for example with a cysteine residue in the antigen through the use of a suitable cross-linking agent as described above. In a specific embodiment, the coat protein is fused with a short peptide sequence of glycine and lysine residues. In another embodiment, the peptide comprises the sequence: GGKGG.

In a further embodiment of the present invention, the antigen is attached via an affinity moiety present on the coat protein. In accordance with this embodiment, the PapMV VLP comprises an affinity moiety, such as a peptide, that is exposed on the surface of the VLP following self-assembly, and which is capable of specifically binding to the antigen. The affinity moiety may be genetically fused (in the case of a peptide or protein fragment), or covalently or non-covalently attached to the PapMV or VLP. Binding of the antigen to the affinity moiety should not interfere with the recognition of the antigen by the host's immune system. In accordance with one embodiment of the present invention, the PapMV VLP comprises an affinity moiety capable of binding a whole protein. In accordance with another embodiment of the present invention, the PapMV VLP comprises an affinity moiety capable of binding a protein fragment or peptide. In accordance with a further embodiment of the present invention, the PapMV VLP comprises an affinity moiety capable of binding a superficial bacterial antigenic component (SBAC).

In a specific embodiment of the present invention, there is provided an APS comprising a PapMV VLP that includes an affinity peptide that binds to a porin protein. In another embodiment of the present invention, there is provided an APS comprising a PapMV VLP that includes an affinity peptide that binds to OmpC or OmpF.

Examples of suitable affinity moieties include, but are not limited to, antibodies and antibody fragments (such as Fab fragments, Fab′ fragments, Fab′-SH, fragments F(ab′)₂ fragments, Fv fragments, diabodies, and single-chain Fv (scFv) molecules), streptavidin (to bind biotin labelled antigens), affinity peptides or protein fragments that specifically bind the antigen.

Suitable peptides or antibodies (including antibody fragments) for use as affinity moieties can be selected by art-known techniques, such as phage or yeast display techniques. The peptides can be naturally occurring, recombinant, synthetic, or a combination of these. For example, the peptide can be a fragment of a naturally occurring protein or polypeptide. The term peptide also encompasses peptide analogues, peptide derivatives and peptidomimetic compounds. Such compounds are well known in the art and may have advantages over naturally occurring peptides, including, for example, greater chemical stability, increased resistance to proteolytic degradation, enhanced pharmacological properties (such as, half-life, absorption, potency and efficacy) and/or reduced antigenicity.

Suitable peptides can range from about 3 amino acids in length to about 50 amino acids in length. In accordance with one embodiment of the invention, the affinity binding peptide is at least 5 amino acids in length. In accordance with another embodiment of the invention, the affinity binding peptide is at least 7 amino acids in length. In accordance with another embodiment of the invention, the affinity binding peptide is between about 5 and about 50 amino acids in length. In accordance with another embodiment of the invention, the affinity binding peptide is between about 7 and about 50 amino acids in length. In other embodiments of the present invention, the affinity binding peptide is between about 5 and about 45 amino acids in length, between about 5 and about 40 amino acids in length, between about 5 and about 35 amino acids in length and between about 5 and about 30 amino acids in length. In accordance with a specific embodiment of the invention, the affinity binding peptide is 3, 4, 5, 6, 7, 8, 9, 10, 11, 12, 13, 14 or 15 amino acids in length. As would be understood by a worker skilled in the art, the length of the peptide selected for binding the antigen to the affinity moiety should not interfere with the ability of the PapMV VLP to self-assemble or the recognition of the antigen, once bound, by the host's immune system.

Affinity moieties comprised by the PapMV or VLP can be single peptides or can be a tandem or multiple arrangement of peptides. A spacer can be included between the affinity moiety and the coat protein if desired in order to facilitate the binding of large antigens. Suitable spacers include short stretches of neutral amino acids, such as glycine. For example, a stretch of between about 3 and about 10 neutral amino acids.

Phage display can be used to select specific peptides that bind to an antigenic protein of interest using standard techniques (see, for example, Current Protocols in Immunology, ed. Coligan et al., J. Wiley & Sons, New York, N.Y.) and/or commercially available phage display kits (for example, the Ph.D. series of kits available from New England Biolabs, and the T7-Select® kit available from Novagen). An example of selection of peptides by phage display is also provided in Example VII, below.

Representative peptides that bind porin proteins identified by phage display are shown in Table 4. One skilled in the art will appreciate that these peptides are examples only and that other peptides having an affinity for a porin of interest can be readily identified using art-known techniques. Truncated versions, for example comprising at least 4 consecutive amino acids, of the sequences set forth in Table 4 that retain the ability of bind a porin protein are also contemplated. In accordance with a specific embodiment of the present invention, there is provided an APS comprising a PapMV VLP that includes one or more affinity peptides comprising all or a part of the sequence set forth in SEQ ID NO:21, SEQ ID NO:22, SEQ ID NO:23, SEQ ID NO:24, SEQ ID NO:25 or SEQ ID NO:26.

TABLE 4
Affinity peptides selected
by phage display and their frequency.

| Target | Selected affinity peptides | SEQ ID NO |
|---|---|---|
| OmpC | SLSLIQT | 21 |
| OmpC | EAKGLIR | 22 |
| OmpC | TATYLLD | 23 |
| OmpF | FHENWPS | 24 |
| OmpF | FHEFWPT | 25 |
| OmpF | FHEXWPT, where X is N or F | 26 |

Preparation of the APS

The present invention provides APSs that comprise PapMV or PapMV VLPs derived from a recombinant PapMV coat protein. The invention further provides recombinant PapMVs VLPs that comprise one or more antigens, or an affinity moiety, in genetic fusion with the coat protein. These recombinant coat proteins are capable of multimerisation and assembly into VLPs. Methods of genetically fusing the antigens, or affinity peptides for linking to antigens, to the coat protein are described below and in the Examples. Methods of chemically cross-linking various molecules to proteins are well known in the art and can be employed.

Papaya Mosaic Virus

PapMV is known in the art and can be obtained, for example, from the American Type Culture Collection (ATCC) as ATCC No. PV-204™. The virus can be maintained on, and purified from, host plants such as papaya (Carica papaya) and snapdragon (Antirrhinum majus) following standard protocols (see, for example, Erickson, J. W. & Bancroft, J. B., 1978, Virology 90:36-46).

PapMV VLPs

The recombinant coat proteins to be used to prepare the VLPs of the present invention can be readily prepared by standard genetic engineering techniques by the skilled worker provided with the sequence of the wild-type protein. Methods of genetically engineering proteins are well known in the art (see, for example, Ausubel et al. (1994 & updates) Current Protocols in Molecular Biology, John Wiley & Sons, New York), as is the sequence of the wild-type PapMV coat protein (see SEQ ID NOs:1 and 2).

Isolation and cloning of the nucleic acid sequence encoding the wild-type protein can be achieved using standard techniques (see, for example, Ausubel et al., ibid.). For example, the nucleic acid sequence can be obtained directly from the PapMV by extracting RNA by standard techniques and then synthesizing cDNA from the RNA template (for example, by RT-PCR). PapMV can be purified from infected plant leaves that show mosaic symptoms by standard techniques (see, for example Example I provided herein).

The nucleic acid sequence encoding the coat protein is then inserted directly or after one or more subcloning steps into a suitable expression vector. One skilled in the art will appreciate that the precise vector used is not critical to the instant invention. Examples of suitable vectors include, but are not limited to, plasmids, phagemids, cosmids, bacteriophage, baculoviruses, retroviruses or DNA viruses. The coat protein can then be expressed and purified as described in more detail below.

Alternatively, the nucleic acid sequence encoding the coat protein can be further engineered to introduce one or more mutations, such as those described above, by standard in vitro site-directed mutagenesis techniques well-known in the art. Mutations can be introduced by deletion, insertion, substitution, inversion, or a combination thereof, of one or more of the appropriate nucleotides making up the coding sequence. This can be achieved; for example, by PCR based techniques for which primers are designed that incorporate one or more nucleotide mismatches, insertions or deletions. The presence of the mutation can be verified by a number of standard techniques, for example by restriction analysis or by DNA sequencing.

As noted above, the coat proteins can also be engineered to produce fusion proteins comprising one or more antigens or affinity peptides fused to the coat protein. Methods for making fusion proteins are well known to those skilled in the art. DNA sequences encoding a fusion protein can be inserted into a suitable expression vector as noted above.

One of ordinary skill in the art will appreciate that the DNA encoding the coat protein or fusion protein can be altered in various ways without affecting the activity of the encoded protein. For example, variations in DNA sequence may be used to optimize for codon preference in a host cell used to express the protein, or may contain other sequence changes that facilitate expression.

One skilled in the art will understand that the expression vector may further include regulatory elements, such as transcriptional elements, required for efficient transcription of the DNA sequence encoding the coat or fusion protein. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding a genetically engineered coat protein. One skilled in the art will appreciate that selection of suitable regulatory elements is dependent on the host cell chosen for expression of the genetically engineered coat protein and that such regulatory elements may be derived from a variety of sources, including bacterial, fungal, viral, mammalian or insect genes.

In the context of the present invention, the expression vector may additionally contain heterologous nucleic acid sequences that facilitate the purification of the expressed protein. Examples of such heterologous nucleic acid sequences include, but are not limited to, affinity tags such as metal-affinity tags, histidine tags, avidin/streptavidin encoding sequences, glutathione-S-transferase (GST) encoding sequences and biotin encoding sequences. The amino acids encoded by the heterologous nucleic acid sequence can be removed from the expressed coat protein prior to use according to methods known in the art. Alternatively, the amino acids corresponding to expression of heterologous nucleic acid sequences can be retained on the coat protein if they do not interfere with its subsequent assembly into VLPs.

In one embodiment of the present invention, the coat protein is expressed as a histidine tagged protein. The histidine tag can be located at the carboxyl terminus or the amino terminus of the coat protein.

The expression vector can be introduced into a suitable host cell or tissue by one of a variety of methods known in the art. Such methods can be found generally described in Ausubel et al. (ibid.) and include, for example, stable or transient transfection, lipofection, electroporation, and infection with recombinant viral vectors. One skilled in the art will understand that selection of the appropriate host cell for expression of the coat protein will be dependent upon the vector chosen. Examples of host cells include, but are not limited to, bacterial, yeast, insect, plant and mammalian cells. The precise host cell used is not critical to the invention. The coat proteins can be produced in a prokaryotic host (e.g., E. coli, A. salmonicida or B. subtilis) or in a eukaryotic host (e.g., Saccharomyces or Pichia; mammalian cells, e.g., COS, NIH 3T3, CHO, BHK, 293, or HeLa cells; insect cells or plant cells).

If desired, the coat proteins can be purified from the host cells by standard techniques known in the art (see, for example, in Current Protocols in Protein Science, ed. Coligan, J. E., et al., Wiley & Sons, New York, N.Y.) and sequenced by standard peptide sequencing techniques using either the intact protein or proteolytic fragments thereof to confirm the identity of the protein.

The recombinant coat proteins of the present invention are capable of multimerisation and assembly into VLPs. In general, assembly takes place in the host cell expressing the coat protein. The VLPs can be isolated from the host cells by standard techniques, such as those described in the Examples section provided herein. The VLPs can be further purified by standard techniques, such as chromatography, to remove contaminating host cell proteins or other compounds, such as LPS. In one embodiment of the present invention, the VLPs are purified to remove LPS.

In one embodiment of the present invention, the coat proteins assemble to provide a recombinant virus in the host cell and can be used to produce infective virus particles which comprise nucleic acid and fusion protein. This can enable the infection of adjacent cells by the infective virus particle and expression of the fusion protein therein. In this embodiment, the host cell used to replicate the virus can be a plant cell, insect cell, mammalian cell or bacterial cell that will allow the virus to replicate. The cell may be a natural host cell for the virus from which the virus-like particle is derived, but this is not necessary. The host cell can be infected initially with virus in particle form (i.e. in assembled rods comprising nucleic acid and a protein) or alternatively in nucleic acid form (i.e. RNA such as viral RNA; cDNA or run-off transcripts prepared from cDNA) provided that the virus nucleic acid used for initial infection can replicate and cause production of whole virus particles having the chimeric protein.

Characteristics of Recombinant and Modified Coat Proteins

Recombinant coat proteins and coat proteins to which antigens or affinity peptides have been attached covalently can be analysed for their ability to multimerize and self-assemble into a VLP by standard techniques. For example, by visualising the purified protein by electron microscopy (see, for example, Example I). In addition, ultracentrifugation may be used to isolate VLPs as a pellet, while leaving smaller aggregates (20-mers and less) in the supernatant, and circular dichroism (CD) spectrophotometry may be used to compare the secondary structure of the recombinant or modified proteins with the WT virus (see, for example, Example I).

Stability of the VLPs, and PapMV, can be determined if desired by techniques known in the art, for example, by SDS-PAGE and proteinase K degradation analyses (see Example II). According to one embodiment of the present invention, the PapMV and PapMV VLPs of the invention are stable at elevated temperatures and can be stored easily at room temperature.

Production of Stock PapMV or VLP

Stocks of recombinant PapMV or VLP can be prepared by standard techniques. For example, PapMV or a pseudovirus comprising the recombinant coat protein can be propagated in an appropriate host, such as Carica papaya or Antirrhinum majus, such that sufficient PapMV or pseudovirus can be harvested.

Stocks of PapMV VLPs can be prepared from an appropriate host cell, such as E. coli transformed or transfected with an expression vector or vectors encoding the recombinant coat protein(s) that makes up the VLP. The host cells are then cultured under conditions that favour the expression of the encoded protein, as is known in the art. The expressed coat proteins will multimerise and assemble into VLPs in the host cell and can be isolated from the cells by standard techniques, for example, by rupturing the cells and submitting the cell lysate to one or more chromatographic purification steps.

As demonstrated in the Examples provided herein, the PapMV VLPs are stable structures and stocks of the VLPs can, therefore, be stored easily at room temperature or in a refrigerator.

Evaluation of Efficacy

In order to evaluate the efficacy of the APSs of the present invention as vaccines, challenge studies can be conducted. Such studies involve the inoculation of groups of a test animal (such as mice) with an APS of the present invention by standard techniques. Control groups comprising non-inoculated animals and/or animals inoculated with a commercially available vaccine, or other positive control, are set up in parallel. After a suitable period of time post-vaccination, the animals are challenged with the appropriate enterobacterium. Blood samples collected from the animals pre- and post-inoculation, as well as post-challenge are then analyzed for an antibody response to the virus. Suitable tests for the antibody response include, but are not limited to, Western blot analysis and Enzyme-Linked Immunosorbent Assay (ELISA).

Cellular immune response can also be assessed by techniques known in the art, including those described in the Examples presented herein. For example, through processing and cross-presentation of an epitope expressed on a PapMV VLP to specific T lymphocytes by dendritic cells in vitro and in vivo. Other useful techniques for assessing induction of cellular immunity (T lymphocyte) include monitoring T cell expansion and IFN-γ secretion release, for example, by ELISA to monitor induction of cytokines (see Example III).

Vaccine Compositions

The present invention provides for compositions suitable for use as vaccines for the treatment or prevention of an enterobacterial infection comprising one or more APS of the invention together with one or more non-toxic pharmaceutically acceptable carriers, diluents and/or excipients. If desired, other active ingredients, adjuvants and/or immunopotentiators may be included in the compositions.

The compositions can be formulated for administration by a variety of routes. For example, the compositions can be formulated for oral, topical, nasal, rectal or parenteral administration, including administration by inhalation or spray. The term parenteral as used herein includes subcutaneous injections, intravenous, intramuscular, intrathecal, infrasternal injection or infusion techniques. In one embodiment of the present invention, the compositions are formulated for topical, rectal or parenteral administration or for administration by inhalation or spray. In another embodiment, the compositions are formulated for parenteral administration.

The compositions preferably comprise an effective amount of one or more APS of the invention. The term “effective amount” as used herein refers to an amount of the APS required to exhibit a detectable immune response. The effective amount of APS for a given indication can be estimated initially, for example, either in cell culture assays or in animal models, usually in rodents, rabbits, dogs, pigs or primates. The animal model may also be used to determine the appropriate concentration range and route of administration. Such information can then be used to determine useful doses and routes for administration in the animal to be treated, including humans. In one embodiment of the present invention, the unit dose comprises between about 10 μg to about 10 mg of coat protein. In another embodiment, the unit dose comprises between about 10 μg to about 5 mg of coat protein. In a further embodiment, the unit dose comprises between about 40 μg to about 2 mg of coat protein. One or more doses may be used to immunize the animal, and these may be administered on the same day or over the course of several days or weeks.

As noted above, the APS of the present invention may comprise a plurality of antigens (in a conjugated and/or non-conjugated form), and a single APS can thus provide a multivalent vaccine formulation. Multivalent vaccines can also be provided through the use of an APS comprising an antigen (in a conjugated and/or non-conjugated form) that is conserved amongst different members of the Enterobacteriaceae. Multivalent vaccine compositions that comprise a plurality of APSs, each APS comprising a different antigen are also contemplated. Multivalent vaccines are useful, for example, to provide protection against more than one member of the Enterobacteriaceae family. Multivalent vaccine formulations include bivalent and trivalent formulations in addition to vaccines having higher valencies. One embodiment of the present invention provides a multivalent vaccine. Another embodiment of the invention provides a multivalent vaccine that comprises an antigen that is conserved across a plurality of enterobacterial species. A further embodiment provides a multivalent vaccine that comprises a plurality of (i.e. two or more) APSs, each APS comprising a different antigen. In a further embodiment of the present invention, there is provided a multivalent vaccine comprising an APS that includes a conjugated OmpC or OmpF antigen and a non-conjugated OmpC or OmpF antigen.

Vaccine formulations comprising a plurality of (i.e. two or more) APSs, each APS comprising a different antigen, can also provide improved protection due to the higher number of epitopes in the formulation. One embodiment of the present invention thus provides for vaccine formulations comprising two or more APSs, each APS comprising a different antigen. In another embodiment, there is provided a vaccine formulation comprising and APS including an OmpC antigen and an APS including an OmpF antigen. In a specific embodiment, the two or more APSs comprised by the vaccine formulation include both conjugated and non-conjugated antigens.

Compositions for oral use can be formulated, for example, as tablets, troches, lozenges, aqueous or oily suspensions, dispersible powders or granules, emulsion hard or soft capsules, or syrups or elixirs. Such compositions can be prepared according to standard methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the APS in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Compositions for oral use can also be presented as hard gelatine capsules wherein the APS is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Compositions for nasal administration can include, for example, nasal spray, nasal drops, suspensions, solutions, gels, ointments, creams, and powders. The compositions can be formulated for administration through a suitable commercially available nasal spray device, such as Accuspray™ (Becton Dickinson). Other methods of nasal administration are known in the art.

Compositions formulated as aqueous suspensions contain the APS in admixture with one or more suitable excipients, for example, with suspending agents, such as sodium carboxymethylcellulose, methyl cellulose, hydropropylmethylcellulose, sodium alginate, polyvinylpyrrolidone, hydroxypropyl-β-cyclodextrin, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Compositions can be formulated as oily suspensions by suspending the APS in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may optionally be added to provide palatable oral preparations. These compositions can be preserved by the addition of an anti-oxidant such as ascorbic acid.

The compositions can be formulated as a dispersible powder or granules, which can subsequently be used to prepare an aqueous suspension by the addition of water. Such dispersible powders or granules provide the APS in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

Compositions of the invention can also be formulated as oil-in-water emulsions. The oil phase can be a vegetable oil, for example, olive oil or arachis oil, or a mineral oil, for example, liquid paraffin, or it may be a mixture of these oils. Suitable emulsifying agents for inclusion in these compositions include naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions can also optionally contain sweetening and flavouring agents.

Compositions can be formulated as a syrup or elixir by combining the APS with one or more sweetening agents, for example glycerol, propylene glycol, sorbitol or sucrose. Such formulations can also optionally contain one or more demulcents, preservatives, flavouring agents and/or colouring agents.

The compositions can be formulated as a sterile injectable aqueous or oleaginous suspension according to methods known in the art and using suitable one or more dispersing or wetting agents and/or suspending agents, such as those mentioned above. The sterile injectable preparation can be a sterile injectable solution or suspension in a non-toxic parentally acceptable diluent or solvent, for example, as a solution in 1,3-butanediol. Acceptable vehicles and solvents that can be employed include, but are not limited to, water, Ringer's solution, lactated Ringer's solution and isotonic sodium chloride solution. Other examples include, sterile, fixed oils, which are conventionally employed as a solvent or suspending medium, and a variety of bland fixed oils including, for example, synthetic mono- or diglycerides. Fatty acids such as oleic acid can also be used in the preparation of injectables.

Optionally the composition of the present invention may contain preservatives such as antimicrobial agents, anti-oxidants, chelating agents, and inert gases, and/or stabilizers such as a carbohydrate (e.g. sorbitol, mannitol, starch, sucrose, glucose, or dextran), a protein (e.g. albumin or casein), or a protein-containing agent (e.g. bovine serum or skimmed milk) together with a suitable buffer (e.g. phosphate buffer). The pH and exact concentration of the various components of the composition may be adjusted according to well-known parameters.

Further, one or more compounds having adjuvant activity may be optionally added to the vaccine composition. Suitable adjuvants include, for example, alum adjuvants (such as aluminium hydroxide, phosphate or oxide); oil-emulsions (e.g. of Bayol F® or Marcol52®); saponins, or vitamin-E solubilisate. Virosomes are also known to have adjuvant properties (Adjuvant and Antigen Delivery Properties of Virosomes, Gluck, R., et al., 2005, Current Drug Delivery, 2:395-400) and can be used in conjunction with an APS of the invention.

As previously noted and demonstrated herein, PapMV and PapMV VLPs have adjuvant properties. Accordingly, in one embodiment of the invention, the vaccine compositions comprise additional PapMV or PapMV VLPs as an adjuvant. In some embodiments, use of PapMV or PapMV VLPs may provide advantages over commercially available adjuvants in that it has been observed that PapMV or PapMV VLPs do not cause obvious local toxicity when administered by injection.

Opsonised vaccine compositions are also encompassed by the present invention, for example, vaccine compositions comprising antibodies isolated from animals or humans previously immunised with the vaccine, antigen or PapMV VLPs. Recombinant antibodies based on antibodies isolated from animals or humans previously immunised with the vaccine, antigen or PapMV VLPs could also be used to opsonise the vaccine composition.

Also encompassed by the present invention are combinations of a vaccine composition comprising an APS of the present invention and a commercially available enterobacterial vaccine. Such vaccines include the Vi and Ty21a typhoid fever vaccines.

Other pharmaceutical compositions and methods of preparing pharmaceutical compositions are known in the art and are described, for example, in “Remington: The Science and Practice of Pharmacy” (formerly “Remingtons Pharmaceutical Sciences”); Gennaro, A., Lippincott, Williams & Wilkins, Philadelphia, Pa. (2000).

Vaccine Stability

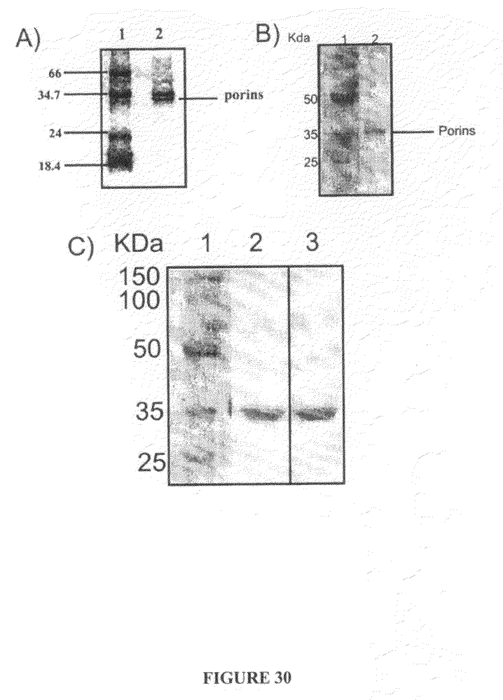

As described above and in the Examples provided herein, PapMV VLPs are stable structures and thus provide for stable vaccine compositions. Porin proteins from S. typhi have also been shown to be stable for long periods of time either under refrigeration or at room temperature (see, for example, Example XIII below and FIG. 30). Thus, one embodiment of the invention provides for a vaccine composition comprising PapMV, or PapMV VLPs and one or more S. typhi porin proteins that is stable for long periods of time, for example, six months or longer, under refrigeration (for example, between about 1° C. and about 10° C.) either in the absence of conventional preservatives or in the presence of a small amount of a suitable detergent. In another embodiment, the invention provides for a vaccine composition comprising PapMV, or PapMV VLPs and one or more S. typhi porin proteins that is stable for at least 6 months and up to about 36 months, under refrigeration. In a further embodiment, the invention provides for a vaccine composition comprising PapMV, or PapMV VLPs and one or more S. typhi porin proteins that is stable for about 5 years under refrigeration. In a further embodiment, the invention provides for a vaccine composition comprising PapMV, or PapMV VLPs and one or more S. typhi porin proteins that is stable for at least 1 month and up to 6 months, at room temperature in the absence of conventional preservatives or in the presence of a small amount of a suitable detergent.

Applications & Uses

The present invention provides for a number of applications and uses of the APSs described herein. Non-limiting examples include the use of the APS as a vaccine against one or more enterobacterial infections, and the use of the APS to screen for antibodies to an enterobacterium. The present invention thus also provides methods for inducing an immune response to one or more enterobacterial antigen(s) in an animal. As well, the use of the APSs of the invention for the preparation of medicaments, such as adjuvants, vaccines and/or pharmaceutical compositions is within the scope of the present invention.

The APS of the present invention is suitable for use in humans as well as non-human animals, including domestic and farm animals. The administration regime for the APS need not differ from any other generally accepted vaccination programs. A single administration of the APS in an amount sufficient to elicit an effective immune response may be used or, alternatively, other regimes of initial administration of the APS followed by boosting with antigen alone or with the APS may be used. Similarly, boosting with either the APS or antigen may occur at times that take place well after the initial administration if antibody titres fall below acceptable levels.

When the APS comprises non-conjugated antigen(s), the PapMV or VLP component can be administered concomitantly with the antigen(s), or it can be administered prior or subsequent to the administration of the antigen, depending on the needs of the subject in which an immune response is desired.

One embodiment of the present invention provides for the use of a vaccine comprising the APS in conjunction with conventional enterobacterial vaccines, such as the Vi or Ty21a typhoid vaccine. In accordance with this embodiment, the APS vaccine may be administered concomitantly with the conventional vaccine (for example, by combining the two compositions), it can be administered prior or subsequent to the administration of the conventional vaccine.

One embodiment of the present invention provides for the use of the APS as an enterobacterial vaccine for humans. Another embodiment of the present invention provides for the use of the APS as an enterobacterial vaccine in humans against one or more species of the genus Escherichia, Salmonella, Yersinia, Shigella, Proteus, Klebsiella or Enterobacter. Yet another embodiment of the present invention provides for the use of the APS as an enterobacterial vaccine in humans against Salmonella spp., E. coli, Yersina spp., Shigella spp., Klebsiella spp. or Enterobacter spp. A further embodiment, provides for the use of the APS as an enterobacterial vaccine in humans against Salmonella spp, E. coli or Shigella spp. The APS for this purpose comprises one or more antigens selected from porin antigens, HSP antigens and SBAC antigens.

Depending on the antigen(s) incorporated into the APS, the vaccines are useful for the treatment and/or prevention of diseases and conditions caused by enterobacteria, such as, for example, typhoid fever, foodborne gastroenteritis, foodborne intestinal infections, urinary tract infections, meningitis, sepsis, bacillary dysentery, plague, enterocolitis, pneumonia, blood stream infections, hospital acquired infections and gram-negative sepsis.

In one embodiment of the present invention, the APS comprises one or more of an OmpC antigen, an OmpF antigen, an antigen from an OmpC orthologue or an antigen from an OmpF orthologue. One embodiment of the present invention provides for the use of an APS comprising one or more OmpC and/or OmpF antigens, or orthologues thereof as an enterobacterial vaccine against various Salmonella spp., E. coli and Shigella spp. In a specific embodiment of the present invention, there is provided the use of an APS comprising OmpC or an orthologue thereof, alone or in combination with an APS comprising OmpF, or an orthologue thereof; as a multivalent vaccine.

Another embodiment of the present invention provides for the use of an APS as a vaccine against S. typhi and for the treatment or prevention of typhoid fever in humans. In one embodiment, the vaccine is suitable for use in both adults and children. In a specific embodiment of the present invention, there is provided an APS comprising at least one antigen derived from a porin protein of S. typhi for use as a human typhoid fever vaccine. The APS can optionally include one or more AlAs that are porin protein antigens, as described above, and/or antigens from different S. typhi bacterial components (i.e. from other proteins such as HSPs, and/or from SBACs). In a specific embodiment, there is provided the use of an APS comprising OmpC or an orthologue thereof, alone or in combination with an APS comprising OmpF, or an orthologue thereof, as a typhoid fever vaccine. In another embodiment of the present invention, there is provided an APS comprising at least one antigen having an amino acid sequence substantially identical to any one of SEQ ID NOs:4, 5, 6, 7, 8, 9, 10, 11, 12, 13, 14, 15, 16, 17, 18, 19 or 20, or a variant thereof, for use as a human typhoid fever vaccine. In a further embodiment, there is provided an APS comprising at least one antigen having an amino acid sequence substantially identical to SEQ ID NOs:4 or 5, or a variant thereof; for use as a human typhoid fever vaccine.

An alternative embodiment of the present invention provides for the use of the APS as an enterobacterial vaccine for non-human animals, such as, cows, pigs, horses, goats, sheep, dogs, cats, chickens, ducks, turkeys, non-human primates, guinea pigs, rabbits, ferrets, rats, hamsters, mice, fish or birds. The vaccines are useful in the treatment and/or prevention of enterobacterial diseases or conditions, such as, respiratory disease, enterocolitis, peritonitis, cystitis and other urogenital infections, systemic colibacillosis, calf scours, edema, mastitis, lymphadenitis, enteritis, septic abortion, metritis, pneumonia, diarrhea, stillbirths, wool damage and septicemia

For use in non-human animals, the APS can comprise one or more of the antigens identified above for human use, including, but not limited to, OmpC antigens, OmpF antigens, HSP antigens and SBAC antigens. In a further embodiment of the present invention there is provided an APS for use as an enterobacterial vaccine against one or more species of medically relevant Enterobacteriaceae in non-human animals. Examples of such bacterial species include, for example, Klebsiella pneumoniae, Yersinia pseudotuberculosis, Shigella spp., E. coli, Salmonella spp. in primates; E. coli, and Proteus spp. in cats and dogs; S. typhimurium, S. newport, S. anatum, and K. pneumoniae in horses; E. coli, Salmonella enterica, Salmonella pullorum and Salmonella gallinarum in birds; E. coli, Salmonella abortusovis, S. typhimurium, and S. dublin in sheep; E. coli, Serratia spp., Klebsiella spp., Citrobacter freundii, Salmonella dublin and S. typhimurium in cows; E. coli, K. pneumoniae, Yersinia enterocolitica, S. choleraesuis, S. typhisuis, Salmonella typhimurium and S. derby in pigs, and Yersinia ruckeri, Edwardsiella tarda and Edwardsiella ictaluri in fish.

As demonstrated herein, PapMV VLPs are capable of potentiating both a humoral and/or a CTL response to an antigen. Accordingly, in one embodiment of the present invention, there is provided a vaccine comprising an APS that includes a S. typhi antigen capable of producing a humoral and/or CTL response.

The present invention also provides for the use of the APS as a screening agent, for example, to screen for antibodies to enterobacteria, such as S. typhi. The APS can be readily adapted to conventional immunological techniques such as an enzyme-linked immunosorbant assay (ELISA) or Western blotting and is thus useful in diagnostic and research contexts.

Kits

The present invention additionally provides for kits comprising one or more APS for use as an enterobacterial vaccine. In one embodiment, the present invention provides for kits comprising one or more APSs including S. typhi antigens for use as a vaccine against typhoid fever. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the vaccine.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

Screening kits containing one or more APS of the invention for use in antibody detection are also provided. The kits can be diagnostic kits or kits intended for research purposes. Individual components of the kit would be packaged in separate containers and, associated with such containers, can be a notice in the form prescribed by a governmental agency regulating the manufacture, use or sale of biological products, which notice reflects approval by the agency of manufacture, use or sale of the biological product. The kit may optionally contain instructions or directions outlining the method of use or administration regimen for the vaccine.

To gain a better understanding of the invention described herein, the following examples are set forth. It will be understood that these examples are intended to describe illustrative embodiments of the invention and are not intended to limit the scope of the invention in any way.

### Examples

### Example I

### Effect of Mutations on RNA Binding and Self-Assembly of PapMV Coat Protein

PapMV coat protein (CPΔN5; shown in SEQ ID NO:3) harbouring the insertion of an alanine at position 2 and in which 5 amino acids at the N-terminus were deleted from the WT sequence was used to create a series of mutants as outlined in Tremblay, M-H., et al., 2006, FEBS J., 273:14-25. Based on the alignment of the amino acids sequences of 19 potexviruses CPs between amino acid 90 to 169 of PapMV CP which revealed that the amino acid corresponding to position 128 of the PapMV CP is an A in most potexviruses but is an E in PapMV, and the report that the charged residues R104, K133, K137, and R161 are likely to be involved in an interaction with the genomic RNA and play an important role in assembly and packaging of the viral genome (Abouhaidar, & Lai, 1989, J. Gen. Virol. 70, 1871-5), the following mutations were made: E128A; K97A; R104K105R108/A; R118D120K121/A; K133K137/A; D142D145/A; E148A; R161A or E166E168/A.

Electron Microscopy and Immunogold Labeling

VLPs or viruses were diluted in 10 mM Tris-HCl pH 8 and were absorbed for 3 minutes on a carbon-coated formvar grid. Grids were blocked with 8 mL of BSA (10 mg/mL) for 30 seconds and washed with PBS. Grids were incubated for 30 minutes at room temperature with a rabbit anti-6XH tag antibody (Amersham, Pittsburgh, Pa., USA) diluted 1:10 in PBS. Grids were then washed three times with PBS and incubated at room temperature for 30 minutes with donkey anti-rabbit antibodies conjugated with 6 nm gold particles (Jackson Immuno Research, West Baltimore Pike, West Grove, Pa., USA) and diluted 1:20 in PBS. Grids were then washed with deionized water and stained as described above.

Circular Dichroism Spectroscopy

CD spectra were recorded on an Olis RSM 1000 (Olis, Conway DriveSuites A & B, Bogart, Ga., USA) rapid scanner monochromator at 20° C. For far UV CD (260-190 nm), thermostated quartz cells of 0.1 cm path length were used. Mean residue ellipticity values ([6]mR_{w} in deg×cmz×dmol) were calculated using the equation: [6]MR_{w}=[6]*MRW/(10×c×1), where [6] is the ellipticity in degrees, MRW is the average molecular weight of the residues in the protein (108 was used in this study), c is the protein concentration in g/ml and 1 is the path length in cm (Johnson, W. C. (1996) Circular Dichroism Instrumentation in Circular Dichroism and the Conformational Analysis of Biomolecules (Fasman, G. D., ed) pp. 635-652, Kluwer Academic/Plenum Publishers).

Near UV CD spectra were recorded (250-350 nm) at RT in a Jasco Model J-710 instrument (Jasco, Commerce Dr. Easton, Md., USA). Recordings were made using a quartz cuvettes (pathlength 0.1 cm). Spectra were averaged from 10 scans of 0.2 nm steps at a rate of 100 nm/min. Rods and Disks samples were respectively at concentration of 1.5 mg/ml and 5.5 mg/ml.

RNA Transcripts and Electrophoretic Mobility Shift Assay (EMSA)

The RNA probe was generated by transcription in vitro using a RiboMAX™ Large Scale RNA Production System-T7 kit (Promega P1300, Madison, Wis., USA) and a clone of 80 nt of the 5′end of PapMV in front of the T7 promoter. The clone was linearised with EcoRI before in vitro transcription. The RNA transcript was purified on a G50 Quick Spin Column for DNA/RNA purification (Roche 1273 965). The same method was used to generate a transcript of the 5′ 1800 nucleotides of PapMV for the in vitro assembly assay. The RNA probe was dephosphorylated using shrimp alkaline phosphatase (Fermentas, Hanover, Md., USA, EFO511) and labelled with gamma ³²P-ATP using T4 polynucleotide kinase (NEB, Ipswich, Mass., USA, MO201 S). The probe was then purified using the G-50 Quick Spin Columns as before. Labelled RNA was incubated with recombinant proteins at room temperature for 60 minutes. 165 fmol of RNA were used for each reaction and various amounts of purified recombinant proteins in the in vitro assembly buffer, which contained 7.5 U of RNase inhibitor (Amersham Biosciences 27-0816O1). The final volume of the reaction was 10 μL; 2 μL of loading dye was added to the sample before loading onto a 5% native polyacrylamide gel. Electrophoresis was performed in 0.5X Tris-borate-EDTA buffer for 90 minutes at 10 mA. The gel was dried and subjected to autoradiography for 16 hours on Kodak BioMax MS film (Amersham Biosciences V8326886) and developed.

Purification of PapMV and Isolation of Disks

PapMV was purified by differential centrifugation from infected papaya leaves that showed mosaic symptoms. Infected leaves (100 g) were ground in 100 mL 50 mM Tris-HCl (pH 8.0) containing 10 mM EDTA in a commercial blender. The ground leaves were filtered through cheesecloth, 1% of Triton X-100 was added to the filtrate, and the filtrate was stirred gently for 10 min. Chloroform was added drop by drop to a volume equivalent to one-quarter of the volume of the filtrate. The solution was stirred for an additional 30 min at 4° C. and centrifuged for 20 min at 10 000 g to remove the precipitate. The supernatant was subjected to high-speed (100 000 g) centrifugation for 120 min. The viral pellet was suspended and subjected to another high-speed centrifugation through a sucrose cushion (30% sucrose) at 100 000 g for 3.5 h. The final viral pellet was suspended in 10 mL of 50 mM Tris (pH 8.0). If color persisted, an additional clarification with chloroform was performed. The purified virus was collected by ultracentrifugation. The isolation of the disks by acetic acid degradation method was performed as described previously (Erickson, et al., 1976, Virology. 72, 514-7).

Trypsin Digest

10 μg of protein was incubated at 37° C. in a volume of 50 μl for 120 minutes in a 100 mM tris HCl buffer pH 8.5 with 0.2 μg of trypsin (Roche, Indianapolis, Ind., USA, 1418475). The reaction was stopped by addition 10 μL of loading dye containing 5% SDS, 5 mM DTT and 40% glycerol. The sample was boiled 5 minutes prior loading on a SDS-PAGE gels. The proteins were visualised by Coomassie blue staining.

Results

The PapMV CP harbours two M residues at positions 1 and 6 of the CP open reading frame (ORF). It is not clear if both of these initiation codons are used during replication of the virus. However, it has been shown that a large proportion of the CP of the purified virus lacks several amino acids at the N-terminus (Zhang, et al., 1993, J. Mol. Biol. 234, 885-7). To ensure production of only one open reading frame in E. coli, the N-terminal 5 amino acids were removed such that M⁶ served as an initiation codon. The introduction of the initiation codon in the NcoI site introduced an extra A that is found in all the constructs. A 6XH tag was added at the C-terminus of the protein to facilitate the purification process. The recombinant protein CPΔN5 was expressed in E. coli BL21 (pLysS) and showed a slightly larger molecular weight (MW) than that of WT CP extracted from purified virus. The difference observed between the two proteins is probably caused by the 6XH tag fusion at the C-terminus. The recombinant protein was affinity purified using a Ni²⁺ column and eluted using 1M imidazole. The yield of the purified recombinant protein was estimated at 40-50 mg/L. Western blot assay using an antibody raised against the WT PapMV CP confirmed that the purified protein was indeed PapMV CP.

Nine different mutants were generated that harbour one, two or three A substitutions. Five mutants R118-D120-K121/A, K133-K137/A, D142-D145/A, R161A and E166-E167-R168/A produced unstable proteins and were undetectable or expressed at very low level. It is likely that mutagenesis in this conserved region affected the native folding of the CP. The mutants K97A, R104K105R108/A, E128A and E148A could be expressed to level similar to CPΔN5 and easily purified using a 6xH tag as shown with the CPΔN5. However, the removal of imidazole during the dialysis made the mutants R104K105R108/A and E148A aggregate and precipitate and it is likely that the mutations affected the folding of the protein.

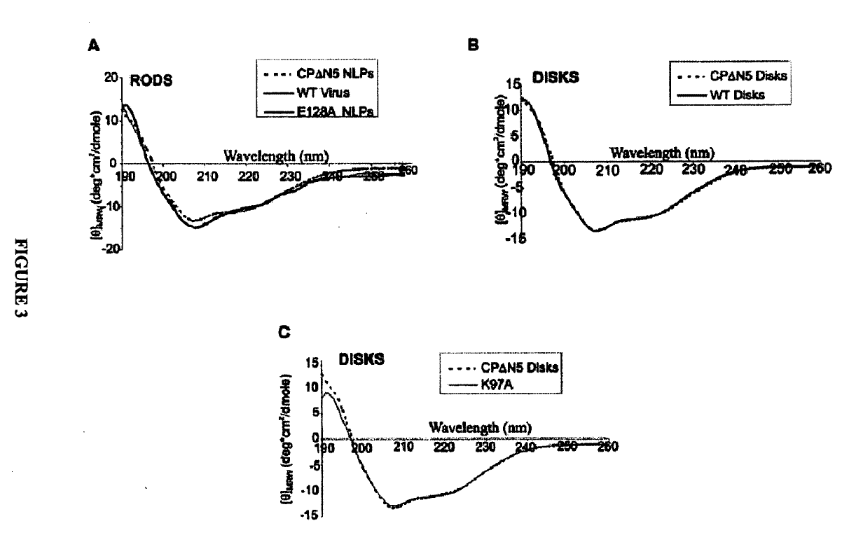

The construct CPΔN5 self assembled into VLPs in E. coli as shown by the electron micrograph of the purified recombinant protein (FIG. 2B). The VLPs were similar in shape and in diameter to the native virus particles (compare FIGS. 2A and 2B). To analyse and quantify the proportion of the purified protein that was found as VLPs, VLPs and smaller aggregates were separated by ultracentrifugation at 100,000 g for 2 hours. Most of the CPΔN5 proteins (80%) were found in the supernatant. VLPs were found in the pellet and account for 20% of the total purified recombinant protein. However, the purified protein K97A remained in the supernatant after ultracentrifugation. On the contrary, the recombinant protein E128A was found totally in the pellet after ultracentrifugation.

Electron microscopy revealed that E128A VLPs isolated from the high speed pellet are similar to the WT virus (FIG. 2C). The length of 150 VLPs for each CPΔN5 and E128A was measured and the average length was determined (FIG. 2F). CPΔN5 VLPs appeared 10 times shorter (50 nm) than the native virus that is 500 nm in length as predicted. However, E128A VLPs are approximately 3 times longer than CPΔN5 VLPs (FIG. 2C and 2F) suggesting that this mutant can more efficiently support the initiation and elongation of assembly. Finally, an electron micrograph of the purified K97A protein revealed disorganised aggregates of 15 to 50 nm in diameter (FIG. 2D). The outline of the aggregates was irregular showing that this protein can not organise itself into VLPs.

The purification of the VLPs using the Ni²+ column was efficient suggesting that the 6XH tag is located at the surface and available for interaction with the affinity column. To confirm this hypothesis, an immunogold labelling experiment was performed on CPΔN5 VLPs using anti-6XHis tag rabbit antiserum followed by a secondary donkey anti-rabbit labelled with gold particles. As expected, the VLPs were decorated with the gold particles (FIG. 2E), thus demonstrating that the fusion of a peptide (6XH) to the C-terminus is tolerated and exposed to the surface of VLPs. This surface exposure of C-terminally fused peptides demonstrates the suitability of the C-terminus as an appropriate point to which to attach an immunogen.

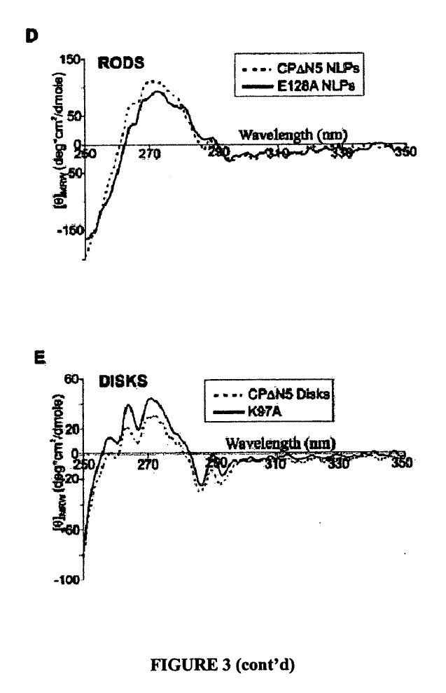

Circular dichroism (CD) spectrophotometry was used to compare the secondary structure of the recombinant proteins with the WT virus. The secondary structure of CPΔN5 was estimated to be 49% α-helices and 15% random coil. The CD spectra of CPΔN5 VLPs and WT virus showed a slightly different profile (FIG. 3A). The CD signal measured at 208 nm was more pronounced for the WT virus than the CPΔN5 VLPs (FIG. 3A). Interestingly, the CD signal measured with E128A VLPs at 208 nm superimposed with the WT virus (FIG. 3A).

The CD signal of the isolated disks (high speed supernatant of the purified protein) of CPΔN5 was identical to isolated disks from the purified virus using the acetic acid method (FIG. 3B). It is interesting to notice that the CD signal measured with disks in general at 208 nm was less pronounced than with VLPs. This result suggests that the content in α-helices is increased when the disks assemble in VLPs. Finally, the folding of the purified protein of K97A was compared with high speed supernatant of CPΔN5. Both proteins showed an identical CD profile (FIG. 3E).

Spectra between 250 and 350 nm were also obtained to measure the absorption of aromatic residues and tryptophan residues in the protein. A change in the environment of those residues affects the signal recorded and indicates variation in the tertiary structure. The VLPs of CPΔN5 and E128A appeared to be similar (FIG. 3D), indicating that the tertiary structures are the same for both VLPs. The spectra of CPΔN5 disks and K97A were very similar which suggests that both proteins have a similar tertiary structure (FIG. 3E). The slight differences in the intensity of the curves between the samples were probably due to a small variation in protein concentrations.

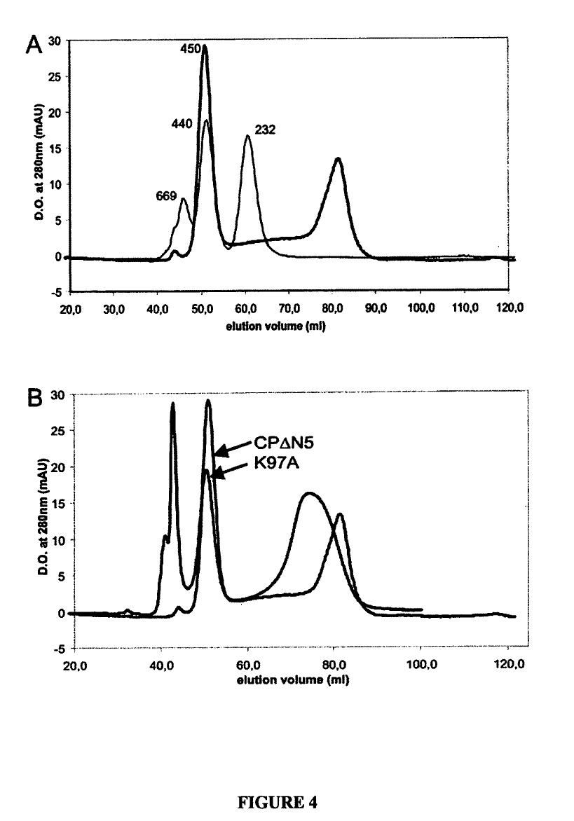

80% of the purified recombinant CPΔN5 and all the K97A proteins were found as multimers (disks) in the supernatant after ultracentrifugation. To measure the level of multimerisation of these proteins, the high speed supernatant of CPΔN5 and the purified protein K97A were analysed using Superdex™ 200. For CPΔN5, most of the proteins were eluted as a high molecular weight complex of 450 kDa (FIG. 4A) which corresponds to a multimer of approximately 20 subunits (molecular weight of the protein subunit is 23 kDa). The second peak eluting at 81.27 ml was collected and loaded on a Superdex™ 75 column to improve the resolution. The protein eluted as a 39 kDa protein. A sample from this peak was submitted to SDS-PAGE and showed a unique band; smaller than CPΔN5 that corresponds to a degradation product of CPΔN5. It is possible that this degraded protein is unable to form a high molecular complex and remains as a dimer in solution.

The elution profile of K97A can be divided into 3 major peaks (FIG. 4B). The second peak was eluted at 50 ml and overlapped with the CPΔN5 disks (FIG. 4A) which probably corresponds to the disk structure. The first peak eluted between 41 and 43 ml and corresponds to aggregated material that is greater than 700 kDa in size. The elution pattern is wide and shows a shoulder that suggests that this material is not uniform and may correspond to an aggregate of K97A disks agglutinated together by non-specific interactions. The third peak was not analysed further and probably corresponds to a truncated protein as shown for the CPΔN5 construct. These results confirm that K97A is able to form disks with other protein subunits but is unable to assemble into VLPs in E. coli.

CPΔN5 disks were isolated from the high-speed supernatant of the purified proteins by affinity chromatography and used for an in vitro assembly assay. Disks of a diameter of 17 nm were isolated by gel filtration. 50 ml of CPΔN5 disks at a concentration of 1 mg/ml were incubated with 0.05 mg of RNA for 30 minutes at room temperature. Electron microscopy demonstrated that the RNA and the protein were assembled into VLPs of regular length (150 nm) that correspond to the length of the RNA (5′ 1800 nt of PapMV) used for the in vitro assembly assay. This result demonstrates clearly that disks of approximately 20 subunits are the building blocks of the VLPs in vitro. The purified K97A recombinant protein failed to assemble the RNA in VLPs under these conditions.

The K97A and E128A mutations showed completely opposite effects on the PapMV CP. To evaluate if CPΔN5 and E128A VLPs contain RNA, the 280/260 ratio of the different VLPs was measured on the spectrophotometer and compared with the proteins of the purified virus (Table 5). As expected, the VLPs showed a smaller 280/260 ratio than disks because of their lower level of RNA. The 280/260 ration of the E128A VLPs was comparable to the purified virus. Interestingly, this ratio was 50% higher with CPΔN5 VLPs. The 280/260 ratio of the isolated disks of CPΔN5 and K97A was comparable to the disks extracted with acetic acid of the purified virus.

TABLE 5
OD 280/260 for PapMV VLPs

| | |
|---|---|
| Virus or VLP | Discs extracted from: |

| Purified | CPΔN5 | E128A | Purified | CPΔN5 | E128A |
|---|---|---|---|---|---|
| PapMV | VLP | VLP | PapMV | VLP | VLP |
| 0.75 | 1.10 | 0.75 | 1.5 | 1.64 | 1.55 |

To evaluate if the ability to make VLPs was directly related to the affinity of CP for RNA, disks from CPΔN5 and the E128A mutants were isolated and compared to K97A. The high speed supernatant of CPΔN5 was used for isolation of the 450 kDa multimer (disks). Since the E128A mutant makes only VLPs in E. coli, these were disrupted using acetic acid treatment and E128A disks were isolated (Abouhaidar & Bancroft, 1978, Virology. 90, 54-9). Different amount of disks were incubated in a volume of 10 μl containing 165 fmol of a ³²P-labelled RNA probe made from a transcript of 80 nucleotides of the 5′ non coding region of PapMV. The protein-RNA complex was separated by an electrophoresis mobility shift assay (EMSA). The disks of CPΔN5 interacted with the probe in a cooperative manner and induced a shift with 500 ng (22 pmol) of proteins. This result shows that the CPΔN5 disks, which are free of RNA after isolation, are able to interact with RNA in vitro only when a molar ratio of 1,000 (disks/RNA) is reached which, corresponds to a weak affinity for RNA. When a similar experiment was performed with the isolated disks of the mutant E128A and the same probe, the E128A disks bound RNA more efficiently than CPΔN5. As little as 50 ng of protein was sufficient to create a protein RNA complex. Purified K97A proteins in the same conditions failed, even at higher concentration (up to 1500 ng) to induce the formation of a protein RNA complex. The EMSA was repeated with RNAs extracted from CPΔN5 VLPs that were labelled as described previously and the same results were obtained with this RNA that do not contain the PapMV packaging signal.

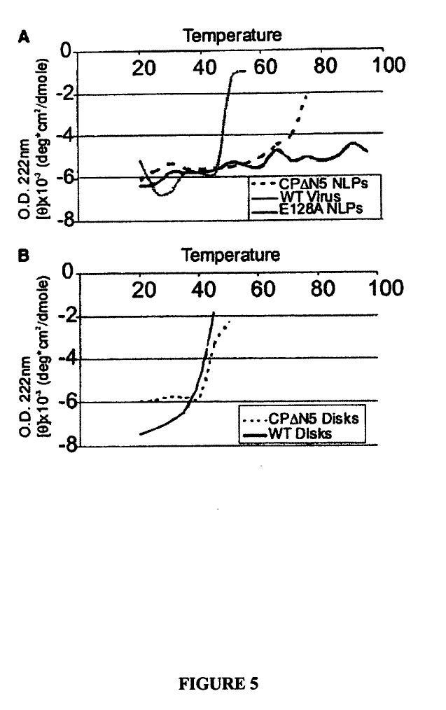

To evaluate the stability of the VLPs and measure if the assembly of the disks into a rod structure improved the stability of the complex, their resistance to heat was monitored by CD spectrophotometry. CPΔN5 VLPs and the virus both resist to high temperature (over 60° C.) before showing any sign of fatigue (FIG. 5A). The purified PapMV was the most stable structure tested and could resist temperatures approaching 100° C. The temperature of inactivation reported for PapMV is 70° C. CPΔN5 VLPs were more sensitive than the WT virus probably due to the presence of the 6xHis tag located at the C-terminus. The E128A VLPs appeared more sensitive to heat and showed sign of fatigue at 42° C. (FIG. 5A). Disks were rapidly denatured at 40° C. (FIG. 5B). This result suggests that the packing of the disks in the rod structure considerably improves stability.

Treatment of PapMV with trypsin results in a cleavage, presumably at amino acid 198 at the C-terminus. Under these conditions, the remaining protein was resistant to the protease. A similar assay was performed on the purified Virion and recombinant VLPs and disks and indicated that PapMV did not seem to be affected by trypsin. Electron microscopy confirmed that treated virus was identical in appearance to untreated virus, however, both the isolated disks from CPΔN5 and the CPΔN5 VLPs were very sensitive to trypsin and several bands of lower molecular weight corresponding to degraded fragments were generated, suggesting that several positively charged residues are exposed and available at the surface of the VLPs. E128A showed similar resistance to trypsin as the WT virus.

### Example II

### Production and Engineering of PapMV gp100 and PapMV Flu VLPs

Cloning of the PapMV CP Gene

The CPΔN5 PapMV coat protein (CP) gene was used in the following Example and was prepared as described in Tremblay, M-H., et al., 2006, FEBS J., 273:14-25. Briefly, the CP gene was amplified by RT-PCR from isolated viral RNA using the following oligonucleotide primers.

Forward CPΔN5 primer:

[SEQ ID NO: 27]

5′-AGTCCCATGGATCCAACGTCCAATCTTCTG-3′
Reverse CPΔN5 primer:

[SEQ ID NO: 28]

5′-ATGCGGATCCTTACTAATGGTGATGGTGATGGTGTTCGGGGGGTGGA
AG-3′

The PCR product was digested with NcoI and BamHI and inserted into the vector pET-3d to generate the CPΔN5 PapMV VLP clone, in which 5 amino acids at the N-terminus were deleted from the WT sequence. PapMV VLP also harbors the insertion of an alanine at position 2 of the recombinant protein. The amino acid sequence of the CPΔN5 PapMV VLP clone is shown in FIG. 1C [SEQ ID NO:3].

Cloning and Engineering of the PapMV gp100 and PapMV Flu Constructs.

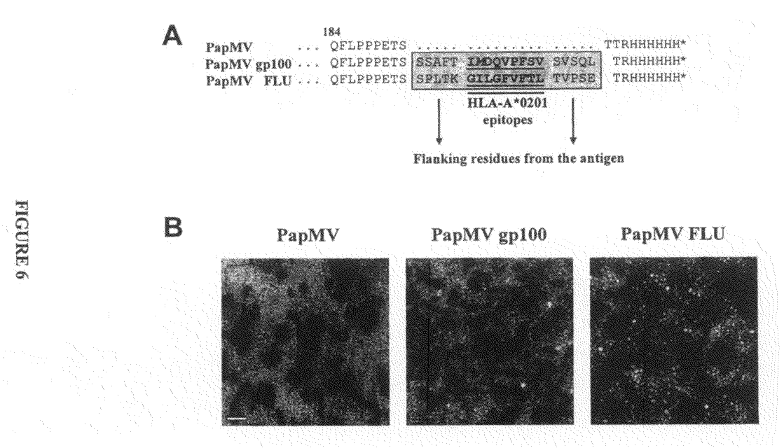

9-mer HLA-A*0201 epitopes from the well-defined tumor antigen gp100 (IMDQVPFSV; SEQ ID NO:51), and from influenza M1 protein (GILGFVFTL; SEQ ID NO:52) were chosen. The HLA-A*0201 epitopes were flanked on the N- and C-terminal sides by 5 residues from the respective native sequences to favour natural processing by the proteasome (see FIG. 6A).

To generate the PapMV gp100 construct, the following oligonucleotides were used:

Sense gp100 oligonucleotide:

[SEQ ID NO: 29]

5′-CTAGTTCTTCTGCGTTCACCATCATGGACCAGGTTCCGTTCTCTGTT
TCTGTTTCTCAGCTGA-3′,
and
Antisense gp100 oligonucleotide:

[SEQ ID NO: 30]

5′-CTAGTCAGCTGAGAAACAGAAACAGAGAACGGAACCTGGTCCATGAT
GGTGAACGCAGAAGAA-3′.

These two oligonucleotides were annealed and cloned into the SpeI and MluI site of the CPΔN5 PapMV CP clone linearized with the same enzymes.

To generate the PapMV FLU construct, the following oligonucleotides were used:

Sense FLU oligonucleotide:

[SEQ ID NO: 31]

5′-CTAGTTCTCCGCTGACCAAAGGTATCCTGGGTTTCGTTTTCACCCTG
ACCGTTCCGTCTGAAA-3′,
and
Antisense FLU oligonucleotide:

[SEQ ID NO: 32]

5′-CTAGTTTCAGACGGAACGGTCAGGGTGAAAACGAAACCCAGGATACC
TTTGGTCAGCGGAGAA-3′.

These two oligonucleotides were annealed and clones at the C-terminus of the CPΔN5 PapMV CP as described above for the gp100 construct.

The resulting PapMV gp100 and PapMV FLU constructs were comprised of the PapMV CP gene with the fusion of the respective peptide at their C-terminus followed by a 6xH tag to ease the purification process. The sequences of the PapMV clones were confirmed by DNA sequencing.

Expression of PapMV, PapMV FLU and PapMV v100 in E. coli

The E. coli expression strain BL21(DE3) RIL (Stratagene, La Jolla, Calif.) was transformed with the plasmid pET-3d containing one of the constructs described above and maintained in 2xYT medium containing ampicillin (50 μg/mL). Bacterial cells were grown at 37° C. to an optical density of 0.6 at 600 nm and protein expression was induced with 1 mm isopropyl-b-d-thiogalactopyranoside (IPTG). Induction was continued for 16 h at 25° C. Bacteria were harvested by centrifugation for 15 min at 6000 r.p.m. The pellet was resuspended in ice-cold lysis buffer (50 mm NaH₂PO₄ pH 8.0, 300 mm NaCl, 10 mm imidazole, 20 μm phenylmethanesulfonyl fluoride, 1 mg/mL lysosyme) and bacteria were lysed by one passage through a French Press. The lysate was centrifuged twice for 30 min at 13 000 r.p.m. to eliminate cellular debris. The supernatant was incubated with 1 mL Ni-NTA (Qiagen, Valencia, Calif.) under gentle agitation for 4 h at 4° C. Lysates were loaded onto a column and the beads were washed with 3×15 mL washing buffer (50 mm NaH₂PO₄ pH 8.0, 300 mm NaCl) containing increasing concentrations of imidazole (10 mm, 20 mm and 50 mm). The beads were then washed with 15 mL working buffer (10 mm Tris/HCl pH 8 or 10 mm sodium phosphate buffer pH 7.2). 2 washing steps were conducted to remove the LPS contaminants, one with 10 mM Tris-HCl, 50 mM imidazole, 0.5% Triton X100 pH 8, and another one with 10 mM Tris-HCl, 50 mM imidazole, 1% Zwittergent pH 8. Proteins were eluted in working buffer containing 1 M imidazole. The eluted proteins were subjected to a high speed ultracentrifugation (100 000 g) for 120 min in a Beckman 50.2 TI rotor. VLPs pellets were resuspended in endotoxin-free PBS (Sigma). Finally, protein solutions were filtered using 0.45 uM filters. The protein concentrations were evaluated by BCA protein kit (Pierce). The level of LPS in the purified proteins were evaluated with the Limulus test under manufacturer's instructions (Cambrex) and was below 0.005 Endotoxin Units (EU)/μg of protein. This procedure yielded more than 20 mg of purified VLPs per liter of bacterial culture.

Electron Microscopy and SDS-PAGE

The proteins were diluted in PBS and absorbed for 3 min on a carbon-coated formvar grid. The grids were washed 2 times with deionized water and stained with uranyl acetate 0.1% during 10 min at room temperature. The grids were then observed on an on a Jeol JEM220FS transmission electron microscope. The average length of 100 VLPs was evaluated using the Adobe Photoshop software.

SDS-PAGE analyses were performed as described in the art (Lepage and Lapointe, 2006, Cancer Res. 66:2423-2432) using the mini-protean system from Bio-Rad (Hercules, Calif.). Proteins were revealed by Coomassie blue staining (Bio-Rad). In some experiments, proteinase K (Invitrogen) was added at a final concentration of 13 μg/ml.

Results

Electron microscopy analysis of the different PapMV VLPs produced in E. coli (FIG. 6B) revealed the typical long rod-shaped structure ranging from 80 to 200 nm in length and a diameter of 15 nm for PapMV VLPs and 16 nm for the engineered PapMV gp100 and Flu VLPs. The PapMV CP was able to spontaneously assemble into VLPs in E. coli that are similar in size and shape to the PapMV VLPs.

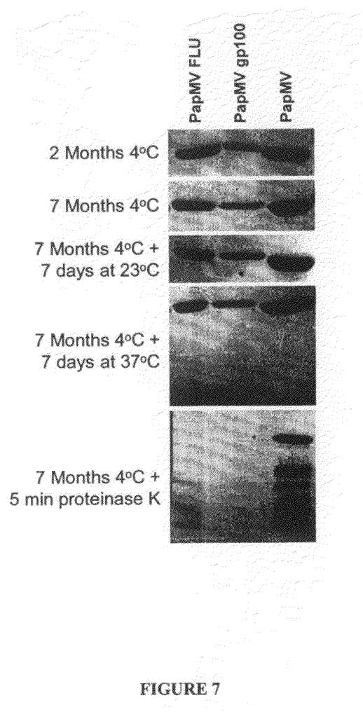

SDS-PAGE analysis was performed on fresh and 7 months old VLPs preparations in PBS at 4° C. (FIG. 7). No evidence of degradation was detected on the gel. Furthermore, the preparations were incubated for an additional 7 days at room temperature or at 37° C., without any noticeable degradation. Finally, the PapMV preparations were incubated with proteinase K as a positive control for degradation, which resulted in the rapid degradation of the engineered PapMV VLPs. The PapMV VLP without a fusion peptide was more resistant to proteinase K suggesting that the fusion at the C-terminus probably locally destabilizes this region and increases susceptibility to this enzyme.

### Example III

### In Vitro Processing and Cross-Presentation of the gp100 and Influenza M1 Epitope Expressed on PapMV VLPs

Peptides

The gp100 and the Flu peptides were synthesized by GLBiochem Shangai LTD and resuspended in a DMSO (Sigma).

Media and Cell Culture

T lymphocytes, dendritic cells (DC), and CD40-stimulated B lymphocytes (CD40-B) were cultured as described in the art (Lapointe et al., 2003, Can. Res. 63:653-662) in complete medium (Iscove's Modified Dulbecco's Medium; Invitrogen; Carlsbad, Calif.; and Wisent; St-Bruno, Qudbec, Canada) supplemented with 7.5% human serum (heat-inactivated, prepared from normal donors), 2 mM L-glutamine, 100 U/ml penicillin/streptomycin and 10 μg/ml gentamicin (the last 3 from Invitrogen and Wisent).

CD40-activated B cells were expanded and cultured from peripheral blood mononuclear cells (PBMC) as described in the art (Lapointe et al., 2003, Can. Res. 63:653-662; Lepage and Lapointe, 2006, Cancer Res. 66:2423-2432) by addition of 500 ng/ml of a soluble trimeric CD40L (Immunex Corporation; Seattle, Wash.) and 500 U/ml recombinant human IL-4 (Peprotech; Rocky Hill, N.J.).

DCs were generated from PBMC collected by apheresis preparations from normal donors (Lapointe et al, 2000, Eur. J. Immunol. 30:3291-3298), by modifying the original protocol described by Sallusto et al., 1994, J. Exp. Med. 179:1109-1118. Briefly, PBMC were enriched from blood by centrifugation on a lymphocyte separation medium (Wisent). Monocytes were enriched following 2 hours adherence in tissue culture flasks or plates at 37° C. (3×10⁷ cells in T-25, 1.5×10⁷ cells/well in 6 well flat bottom plates or 5×10⁶ cells/well in 24 well flat bottom plates, all from Costar, Corning, N.Y.). Adherent cells were washed once with PBS (Wisent) and then cultured in complete medium supplemented with 100 ng/ml of GM-CSF (1,000 U/ml) and 500 ng/ml of IL-4 (1,000 U/ml) (both from Peprotech, Rocky Hill, N.J.). GM-CSF and IL-4 were added again on days 3 and 5. PapMV VLPS (prepared as described in Example II) were added on day 6 and harvested at day 7 for recognition and expansion experiments.

The melanoma cell line 1088mel was established at the Surgery Branch (NCI/NIH). SK23, T2, and breast tumor cell lines MDA231 were obtained from the American Type Culture Collection (ATCC; Manassas, Va.). All tumor cell lines were cultured in RPMI 1640 medium supplemented with 10% heat-inactivated FBS, 2 mM L-glutamine, 100 U/ml penicillin/streptomycin and 10 μg/ml gentamicin.

Cross-Presentation from PapMV Pulsed APC

Different target cells were analyzed for MHC class I presentation of defined epitopes with gp100- or Influenza M1-specific T cells. Gp100-specific CD8⁺ T cell clones were kindly provided by the National Cancer Institute; NIH, Bethesda, Md. and were specific to both the native HLA-A*0201-restricted epitope at position 209-217 (ITD QVP FSV; SEQ ID NO:53), and to the modified version with an M at position 210 (IMD QVP FSV; SEQ ID NO:51), which enhanced the stability of the peptide/MHC complex. Gp100-specific T cells were expanded using the rapid expansion protocol described by Dudley et al., 1999, J. Immunother, 22:288-298.

T cell lines specific to the Influenza M1-derived HLA-A*0201-restricted epitope (59-67; GIL GFV FTL; SEQ ID NO:52) were generated as follow. PBMC from HLA-A*02⁺ normal donors were identified by flow cytometry (FACScalibur; BD Biosciences, Mississauga, ON) with a specific antibody (OneLambda, Canoga Park, Calif.). PBMC prepared as described above, were stimulated in multiple wells of 48 well plates with 1 μM of synthetic FLU peptide in medium described above. Cultures were re-stimulated 7 days later with either peptide pulsed (1 μM for 3 hours followed by 3 washes in PBS) autologous PBMC or CD40-stimulated B lymphocyte cultures. Interleukin (IL)-2 (Chiron, Emeryville, Calif.) was then added every 2-3 days at 150 IU/ml and cultures were kept between 0.5 and 2×10⁶ cells/ml. Specificity of individual cultures was assessed by interferon (IFN)-γ secretion assays by ELISA with coupled antibodies (Endogen; Woburn, Mass.) after co-culture with peptide pulsed T2 cells as described in the art (Lapointe et al., 2001, J. Immunol. 167:4758-4764).

To evaluate cross-presentation mediated by PapMV CP, CD40-activated B cells or DC were pulsed with various versions of the modified PapMV CP at 10-50 μg/ml for 20 hours. Cells were harvested, washed twice with PBS, and seeded in complete media (at 4-10×10⁴ cells/well) in 96 well-plates. Gp100- or Influenza M1-specific T cells were added at 2-10×10⁴ cells/well in complete media for 20 hours. Culture supernatants were harvested and interferon (IFN)-γ was evaluated by ELISA as described in the art (Lepage and Lapointe, 2006, Cancer Res. 66:2423-2432). In some experiments, APCs were pre-treated for 1 hour with 50-70 μM chloroquine (Sigma), or 20-25 μg/ml lactacystin or 1.3-3.3 μM MG-132 (the last 2 from Calbiochem, San Diego, Calif.). Cells were washed with PBS and re-suspended in media containing 1/20 of the original inhibitor concentration. Treated cells were pulsed with the different PapMV variants and analysis of MHC class I mediated presentation was performed as described above.

T Cell Expansion

CD40-activated B cells or DC were pulsed with various versions of the PapMV CP (as described in Example II above) for 20 hours. Cells were harvested and washed twice with PBS. Pulsed APC (2-5×10⁵) were co-cultured with autologous PBMC at 2×10⁶ cells in 500 μl of complete media, in single wells of a 48 well-plate. When media turned yellow, 200 μl of medium was removed, and 400 μl fresh complete media was added. On day 7 to day 10, freshly PapMV-pulsed APC (5×10⁵; pulsed for 20 hours and washed twice in PBS) were added to individual cultures. IL-2 was then added at 100 IU/ml and every 3 days.

T cell specificity was then assessed on day 15 to 20. Briefly, expanded cells were co-cultured with peptide pulsed T2 cells as described in the art (Lapointe et al., 2001, J. Immunol. 167:4758-4764), or PapMV-pulsed APC. IFN-γ secretion was then evaluated by ELISA with coupled antibodies (Endogen; Woburn, Mass.) as described in the art (Lepage and Lapointe, 2006, ibid.). Alternatively, the frequency of antigen-specific T cells was assessed by ELISPOT assay, using coupled antibodies (MABTECH) according to manufacturer's instructions. Spots were enumerated with an automated counter (CU Technologies, Cleveland, Ohio).

Results

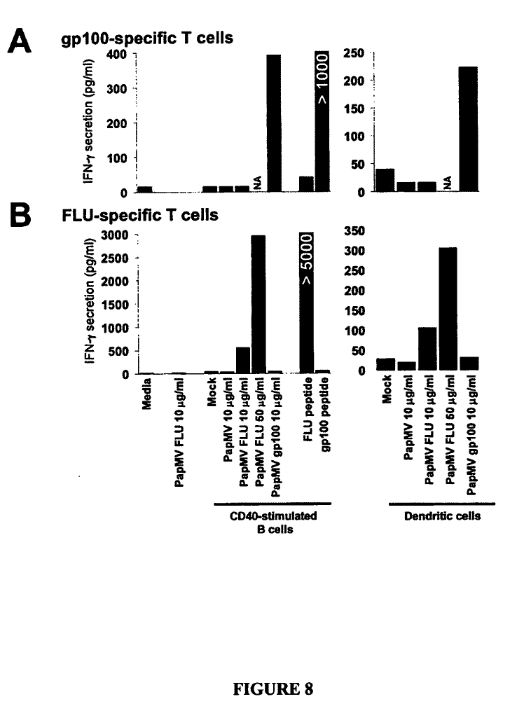

DC are defined in the art as the optimal APC, and it has been demonstrated in the current experiment and in the art that B lymphocytes expanded after CD40 stimulation are efficient APC. These APC were pulsed with the PapMV VLPs, PapMV gp100 and PapMV-Flu and co-cultured with defined T cells specific to MHC class I epitopes from gp100 and Influenza M1 proteins. As presented in FIG. 8A, only APC pulsed with PapMV gp100 were recognized by the gp100-specific T cell clone. Conversely, only PapMV Flu-pulsed APC were recognized by the Influenza M1 specific T cells (FIG. 8B). The specificity of each T cell cultures was confirmed by pulsing CD40-activated B cells with the synthetic peptides corresponding to each epitopes. Also, the addition of PapMV-Flu to the specific T cells failed to stimulate them to secrete IFN-γ, indicating that APC are essential for peptide recognition.

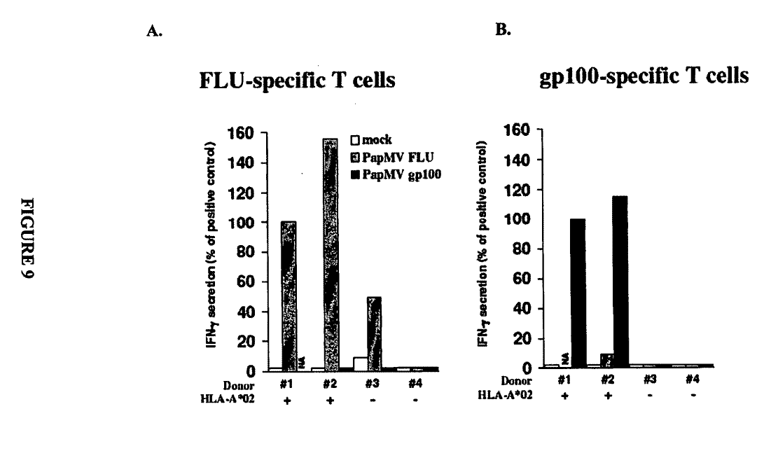

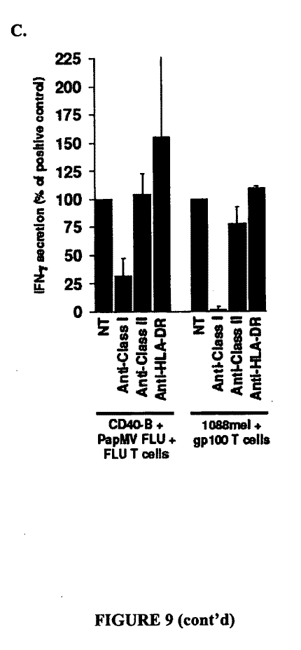

To confirm that HLA-A*02 was the restriction element involved in peptide presentation, similar experiments were performed with APC prepared from 2 additional HLA-A*02 donors, and 2 others negative for this allele. As shown in FIG. 9A, the FLU-specific T-cells were mostly reactive with PapMV-Flu pulsed on HLA-A*02⁺ donors (left panel). The weak reactivity with donor #3 may be explained by the fact that the FLU T-cell line was heterogeneous, and T-cells specific to the peptide, potentially presented by another MHC class I allele, may be present in the culture. Moreover, the gp100-specific T-cell clone was reactive only with PapMV gp100-pulsed HLA-A*02⁺ APC (FIG. 9A; right panel), further confirming that APC expressing the relevant restriction element are necessary for antigenic presentation. Finally, presentation by MHC class I was controlled using antibodies blocking either MHC class I, or class II or HLA-DR presentation, as performed in the art (Lapointe et al., 2003, Can Res. 63:2836-2843; Lapointe et al., 2001, J. Immunol. 167:4758-4764). As a control, a melanoma line expressing both HLA-A*0201 and gp100 was co-cultured with the gp100-specific T-cell clone, and only antibody blocking MHC class I presentation abrogated the recognition, as expected (FIG. 9B; right section). Co-culture of gp100-specific T-cell clones with lines either HLA-A*02⁻/gp100⁺, or +/−, or −/− failed to provoke IFN-γ secretion as demonstrated in the art (Dudley et al., 1999, J. Immunother. 22:288-298; Lapointe et al., 2001, ibid.). When the panel of blocking antibodies was applied to the PapMV Flu-pulsed APC, only the anti-MHC class I decreased the recognition by the specific T cell line (FIG. 9B; left section). These last data demonstrate that the FLU peptide derived from PapMV-Flu was presented by MHC class I, specifically HLA-A*02.

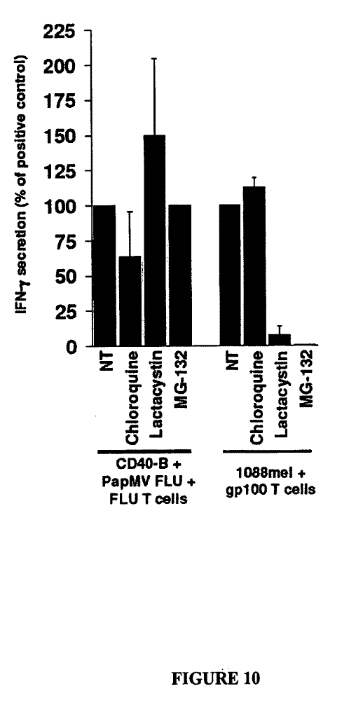

In order to determine whether the processing of the PapMV VLPs was mediated by the proteasome, two different proteasome inhibitors were exploited, namely, lactacystin and MG-132, and their activities controlled by blocking classical MHC class I presentation of the HLA-A*0201 epitope from gp100 by melanoma cells (FIG. 10, right section). When using PapMV Flu-pulsed APC, the presentation of the M1 peptide was unaffected by both inhibitors (FIG. 10, left section). The pre-treatment with chloroquine, which neutralizes the pH of endosomes, had a weak negative effect on the cross-presentation of the FLU epitope, while similar treatment did not change the classical MHC class I presentation of the melanoma cells, as expected. Overall, these data suggests that the MHC class I cross-presentation mediated by the PapMV VLPs is proteasome-independent.

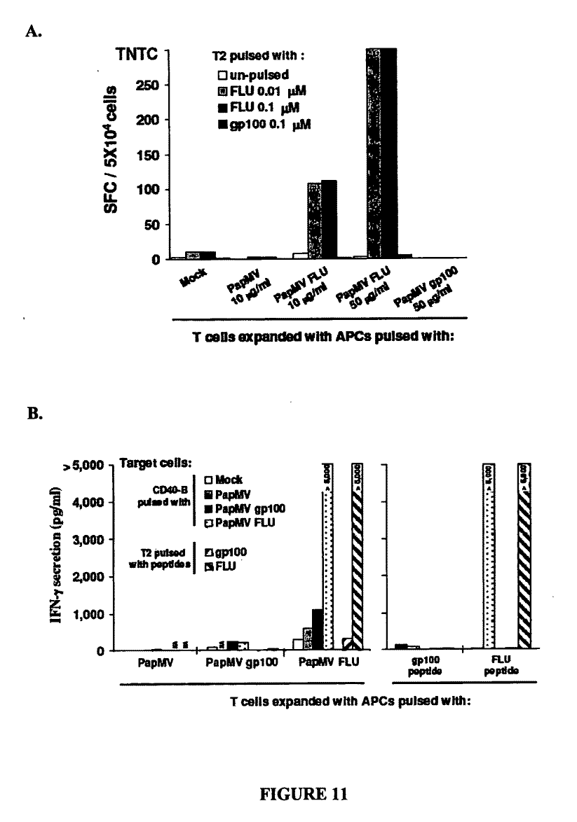

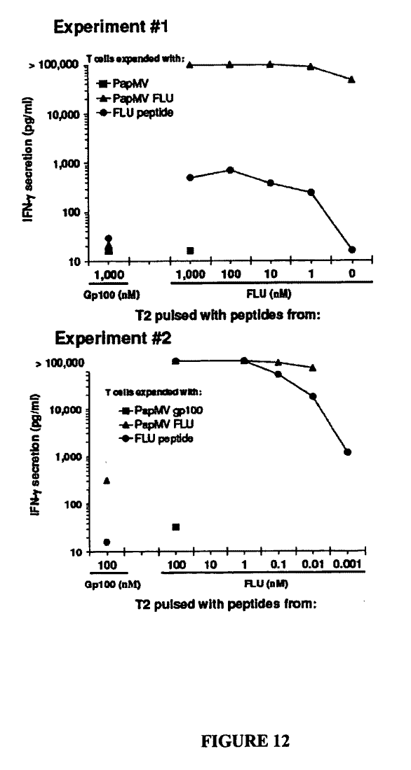

Also evaluated was whether APC pulsed with the PapMV VLPs would have the capacity to expand antigen-specific T lymphocytes from an heterogeneous T cell population from PBMC. DC and CD40-activated B lymphocytes were pulsed with PapMV VLPs and pulsed APC were co-cultured with autologous PBMC according to the protocol described above. The frequency of specific expanded T cells was first evaluated by ELISPOT assay. As shown in FIG. 11A, cells specific to HLA-A*0201 Influenza M1 peptide were generated when PapMV Flu-pulsed APC were used with PBMC. Un-pulsed APC, or the one pulsed with PapMV or PapMV gp100 failed to generate Flu specific T cells, as expected. No gp100-specific T cells were generated, as expected for a healthy normal donor with no melanoma. Expanded T cells were next evaluated for reactivity against various pulsed APC, and IFN-γ secretion seemed equally high when T cells expanded with APC pulsed either with the FLU peptide or PapMV Flu were used to expand T cells (>5000; FIG. 11B). APC pulsed with either PapMV, PapMV gp100 or PapMV Flu failed to generate T cells specific to the PapMV CP, suggesting that cellular pre-immunity to the PapMV CP is marginal. Finally, expanded T cells were co-cultured with T2 cells pulsed with different amount of the HLA-A*0201 Influenza M1 peptide (FIG. 12). In two independent experiments, highly reactive T cells with high avidity were generated, since T cells had the capacity to recognize T2 cells pulsed with 0.1 and 0.01 nM of the peptide. Secretion was mostly >100 000 pg/ml, which is very high, and T cells raised with APC pulsed with the Influenza M1 peptide were generally less avid. From these experiments, it is concluded that PapMV-pulsed APC had the capacity to expand specific T cells with high avidity. Interestingly, there is no evidence of pre-existing cellular pre-immunity to the PapMV CP.

### Example IV

### Production and Engineering of PapMVCP-E2 and PapMVCP₂₇₋₂₁₅-E2

This Example demonstrates that multimerisation of the PapMV coat protein is essential for the immunogenicity of the PapMV VLPs.

Cloning and Engineering of the PapMV Coat Protein

The PapMV CP gene was cloned as described in Example IV. To generate the PapMVCP-E2 construct, the following oligonucleotides were used:

[SEQ ID NO: 33]

5′-GATCACTAGTGTGGTGGTGGGTACCACCGATCGTAGCGGTGCGCCGA
CCTACAGCTGGGGTGCGAACGATACGCGTCATG-3′,
and

[SEQ ID NO: 34]

5′-CATGACGCGTATCGTTCGCACCCCAGCTGTAGGTCGGCGCACCGCTA
CGATCGGTGGTACCCACCACCACACTAGTGATC-3′.

These two oligonucleotides were annealed together and digested with SpeI and MluI before ligation into the SpeI/MluI-linearized PapMVCP clone.

The expression vector for the truncated coat protein E2 fusion, PapMVCP27-215-E2, was constructed from the PapMVCP-E2 plasmid by first preparing the following two oligonucleotides (including an Ncol restriction site) designed to delete the 26 first amino acids of the PapMV CP were used for PCR:

Forward primer:

[SEQ ID NO: 35]

5′-AGTCCCATGGCCGATCCAACGTCCAATCTTCTG-3′,
and
Reverse primer:

[SEQ ID NO: 36]

5′-ACGTCCATGGTATATCTCCTTCTTAAAG-3′.

The PCR product was then self-ligated. The expression vector for PapMVCP27-215 was derived from the PapMVCP plasmid following the same procedure as for the construction of the PapMVCP27-215-E2 clone. The sequences of all PapMV clones were confirmed by DNA sequencing.

PapMVCP-E2 and PapMVCP27-215-E2 Expression and Purification

Expression and purification of PapMVCP constructs were performed as described in Example IV. The E2 peptide was synthesized by GLBiochem Shangai LTD and resuspended in a endotoxin free PBS (Sigma). Protein solutions were filtrated using 0.45 μM filters before use. The amount of protein was evaluated using a BCA protein kit (Pierce). The level of LPS in the purified protein was evaluated with the Limulus test according to the manufacturer's instructions (Cambrex) and was below 0.005 endotoxin units (EU)/μg of protein.

SDS-PAGE, Electroblotting and Electron Microscopy

SDS-PAGE and electroblotting were performed as described in the previous examples. Proteins were diluted in PBS and were absorbed for 3 min on a carbon-coated formvar grid. The grids were washed twice with deionised water and stained with 0.1% uranyl acetate for 10 min at room temperature. The grids were then observed on a Jeol JEM220FS transmission electron microscope. Average VLP length was evaluated by measuring 100 VLPs using Adobe Photoshop software.

Immunization

Five 4- to 8-week-old C3H/HeJ mice (Charles Rivers Laboratories) were injected subcutaneously with 25 μg of PapMVCP-E2, PapMVCP27-215-E2 or the equivalent amount of the E2 peptide (2 μg) or endotoxin-free PBS (Sigma). Primary immunization was followed by one booster dose given 2. weeks later. Blood samples were obtained at different time points and stored at −20° C. until analysis. All the experimental protocols were approved by the Laval University animal protection committee.

ELISA Quantification

Costar High Binding 96-well plates (Corning, N.Y., USA) were coated overnight at 4° C. with 100-200 μl/well of P3, P3E2, PapMVCP, PapMVCP27-215, or PapMVCP-E2 diluted to a concentration of 1 μg/ml in 0.1 M NaHCO₃ buffer pH 9.6. The plates were blocked with PBS/0.1% Tween-20/2% BSA (150 μl/well) for 1 hour at 37° C. After washing three times with PBS/0.1% Tween-20, sera were added in 2-fold serial dilution beginning from 1:50 and incubated for 1 hour at 37° C. Following incubation, the plates were washed three times and incubated with 100 μl of peroxidase-conjugated goat anti-mouse IgG, IgG1, IgG2a, IgG2b (all from Jackson Immunoresarch), IgG3 (Rockland) at a dilution of 1/10,000 in PBS/0.1% Tween-20/2% BSA for 1 hour at 37° C. After three washes, the presence of IgG was detected with 100 μl of TMB-S according to the manufacturer's instructions; the reaction was stopped by adding 100 μl of 0.18 mM H₂SO₄ and the OD was read at 450 nm. The results are expressed as antibody endpoint titer, determined when the OD value is 3-fold the background value obtained with a 1:50 dilution of serum from PBS mice.

For the determination of antibody levels in human sera, the same conditions were applied, except that the peroxidase-conjugated goat anti-human IgG as secondary antibodies were used at a dilution of 1/80000. Sera from infected HCV patients were provided by B. Willems (Hopital Saint Luc, CHUM): the results are expressed as antibody endpoint titer, defined as when the OD value is 3-fold the background value obtained with a 1:25 dilution of serum from a pool of sera from 15 non-infected patients.

Splenocyte Restimulation

CD-1 mice (22 weeks old) were immunized with PapMVCP-E2 (25 μg) on days 0, 15, 30, 45 before being sacrificed on day 65. Spleens were removed and suspended in DMEM (2×10⁵ cells/well). Red blood cells were removed with hypertonic ammonium chloride solution. Splenocytes were washed and resuspended in 200 μl of DMEM medium (DMEM supplemented with 10% FBS [HyClone], 100 U/ml penicillin, 100 μg/ml streptomycin, 2 mM L-glutamine, 1 mM sodium pyruvate, and 50 μM β-mercaptoethanol) to a concentration of 2.5×10⁵ cells/ml in 96-well flat-bottom microplates (Costar). Samples were incubated four days with 25 μg/ml of PapMVCP or PapMVCP-E2 VLPs. Concavalin A (ConA—10 μg/ml) and PBS were used as positive and negative controls. Cells culture supernatants were collected and cytokines were measured using the liquid mouse 10 cytokines kit (Qiagen).

Air Pouch in Mice

Air pouches were raised in 10- to 12-week-old CD-1 mice (Charles River Laboratories). Air pouches were raised on the dorsum by subcutaneous injection of 3 ml of sterile air on days 0 and 3. On day 7, one ml of recombinant PapMVCP (1 to 10 μg/ml), LPS (10 μg/ml) or PBS were injected in the air pouches. Six hours after the treatment, the mice were killed by asphyxiation using CO₂. The air pouches were washed once with 1 ml PBS-5 mM EDTA, and then twice with 2 ml of PBS-5 mM EDTA, and the exudates were centrifuged at 500×g for 5 min at room temperature. Cells were counted with a hematocytometer following acetic blue staining.

Bone Marrow Cell Extraction and Differentiation in APCs

Bone-marrow progenitors cells were obtained from the femurs of BALB/c mice and cultured for 6 days in dendritic cells differentiation bone marrow medium (95% RPMI with 1% penicillin-streptomycin and supplemented with 5% X63-GM-CSF supernatant media culture; the X63-GM247 CSF cell line was provided by B. Ludewig, Research Department, Kantonal Hospital St. Gallen, Switzerland). Medium was partially replaced on day 2 and 4. On day 6, the medium was replaced by medium without LX63 conditioned medium. On day 7, enrichment of APCs was verified by flow cytometry using FITC anti-CD11c and PE-Cy5.5 anti-CD11b surface markers (BD Biosciences). The preparation contained 25% of CD11c⁺Cd11b⁺ cells, and more than 80% of CD11b+ cells. This preparation is referred to herein as APCs.

Flow Cytometry

To evaluate the internalization of the PapMVCP-E2 or PaPMVCP27-215-E2 in APCs, 1 million of bone marrow derived APCs were incubated for 2 hours at 37° C. with either 25 μg of PapMVE2 or PaPMVCP27-215-E2. Briefly, cells were blocked with PBS 10%; FBS and anti-CD16/CD32 (1 μg/1 million cells) 15 min at 4° C. After 2 washes with PBS, cells were fixed with PBS/2% paraformaldehyde for 10 min at room temperature. After 2 washes with permeabilization buffer (PBS 10%; FBS 0.2%; Triton X-100), cells were incubated for 45 min at 4° C. with the rabbit polyclonal antibodies diluted 1/200 in permeabilization buffer. After 2 washes with permeabilization buffer, cells were incubated for 45 min at 4° C. with the secondary antibodies anti-rabbit IgG alexa 488 (Molecular Probes) diluted 1/5000 in permeabilization buffer. After washing with PBS, cells were immediately analysed with an EPICS-XL cytofluorometer. Data analysis was performed using WINMDI2.8. The rabbit polyclonal Ab used for detection was produced in our own facilities: rabbit preimmune serum was used as a negative control.

Confocal Microscopy

APCs were grown (200,000 cells/well) in a 12-well plates (Corning, N.Y., USA) containing sterile slides in the bottom following the same differentiation protocol as described previously. For antigen internalization studies, 5 μg of antigen/200 000 cells was used. The fixation, permeabilization, primary and secondary antibodies incubation steps were as described for flow cytometry. Slides were analysed immediately with a Fluoview Fv300 confocal microscope with a X60 oil immersion objective. Fluorescence images were acquired sequentially to avoid non-specific channel interference and by x-z sectioning. Pictures were then digitally processed with Image J software.

Statistical Analysis

Nonparametric Krustal-Wallis and Dunn's multiple comparison tests were used for statistical analysis. A value of P<0.05 was considered statistically significant. Statistical analyses were performed with the program PRISM 3.03.

Results

The purified recombinant proteins showed the expected molecular weights of 23 kDa (PapMVCP27-215-E2) and 26 kDa for PapMVCP and PapMVCP-E2 and endotoxin levels were always below 0.005 EU/μg of protein. Electron microscopy (EM) observations confirmed that the addition of the E2 peptide at the C-terminus of the PapMVCP did not affect the ability of the protein to self-assemble into VLPs that are similar to the recombinant PapMVCP VLPs. As expected, PapMVCP27-215-E2 was unable to form VLPs and remained as a monomeric form as previously shown (Leclerc et al., 1998, J Biol Chem, 273:29015-21). The lengths of the VLPs are variable, with a size range of 201±80 nm. A 201 nm length protein represents 560 copies of the CP presenting the E2 peptide in a repetitive and crystalline fashion.

To test the pro-inflammatory properties of PapMVCP VLPs, the mouse air pouch model was used. Injection of 10 μg of PapMVCP VLPs with very low LP content (<0.005 EU/μg) failed to induce the recruitment of leucocytes into the pouch of CD1 mice six hours after the treatment. In contrast, injection of LPS at doses of 1,000 and 1 EU was very effective in inducing the recruitment of leucocytes (FIG. 13). This result suggests that PapMVCP VLPs are not pro-inflammatory after 6 hours and that the very low level of LPS in PapMVCP protein samples would not exert any notable immunogenic effects in subsequent experiments.

The capacity of the monomeric (PapMVCP27-215-E2) and the multimeric (PapMVCP-E2) forms to be internalized in bone marrow derived APCs enriched in bone-marrow-derived dendritic cells (BMDDC) was tested. Flow cytometry analysis showed that APCs become efficiently immunolabelled by both the multimeric and the monomeric forms (95.3% for the PapMVCP-E2 VLP and 92.6% for the PapMVCP27-215-E2 VLP). To visualize the interaction between the recombinant proteins and the APCs, the treated APCs were observed by confocal microscopy. In both cases, the immunolabelled PapMVCP signal was clearly vesicular, intracytoplasmic and perinuclear. Both recombinant proteins were efficiently internalized in the APCs.

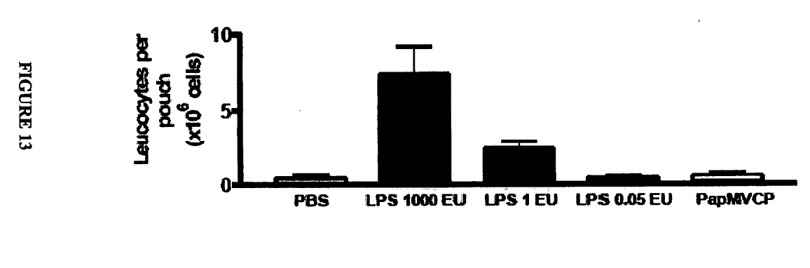

To examine the capacity of PapMVCP VLPs to induce an immune response, C3H/HeJ mice were injected subcutaneously with 25 μg of the recombinant VLPs (PapMVCP-E2) or 25 μg of the monomeric form (PapMVCP₂₇₋₂₁₅-E2). The amount of E2 peptide present in each dose is estimated at 2 μg. A booster dose was given on day 15 after primary immunization. Mice sera were assayed for anti-PapMVCP, PapMVCP₂₇₋₂₁₅ and anti-E2 peptide antibodies. Anti-CP IgG was clearly detected in mice immunized with PapMVCP-E2 on day 12, while only a weak level of anti-CP was detected in the sera of mice vaccinated with PapMVCP₂₇₋₂₁₅-E2, even after the booster on day 15 (FIG. 14).

### Example V

### Adjuvant Effect of PapMV

PapMV was purified as described in Example I.

Antigens

LPS-free OVA Grade VI was purchased from Sigma-Aldrich Chemical Co, St Louis, Mo. Hen egg white lysozyme (HEL) was purchased from Research Organics Inc. Cleveland, Ohio. LPS from E. coli O111:B4 was purchased from Sigma-Aldrich, St Louis, Mo.

Immunizations

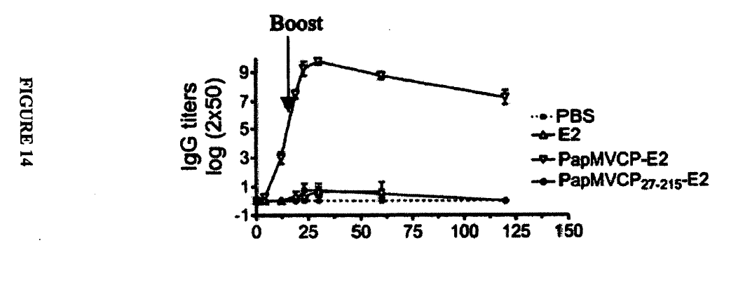

BALB/c mice, 6-8 weeks old, were bred and kept under the animal facilities of the Experimental Medicine Department, Faculty of Medicine, National Autonomous University of Mexico (UNAM), and were cared for in conformity with good laboratory practice guidelines. To study the effects of adjuvant, groups of mice were immunized i.p. on day 0 with 2 mg of OVA or HEL alone or with 30 mg of PapMV, CFA 1:1 (v/v), or 5 mg of LPS from E. coli O111:B4 (Sigma-Aldrich). Control mice were injected with saline solution only. Blood samples were collected from the retro-orbital sinus at various times, as indicated in FIG. 15. Individual serum samples were stored at −20° C. until analysis. Three mice were used in each experiment.

Determination of Antibody Titers by ELISA

High-binding 96-well polystyrene plates (Corning®, New York, N.Y.) were coated with 1 mg/mL of PapMV, 100 mg/mL of HEL, or 150 mg/mL OVA in 0.1 M carbonate-bicarbonate buffer (pH 9.5). Plates were incubated for 1 h at 37° C. and then overnight at 4° C. Before use the next morning, plates were washed three times in PBS (pH 7.2) containing 0.05% Tween-20 (PBS-T) (Sigma-Aldrich). Nonspecific binding was blocked with 5% nonfat dry milk diluted in PBS (PBS-M) for 1 h at 37° C. After washing, mice serum was diluted 1:40 in PBS-M, and 2-fold serial dilutions were added to the wells. Plates were incubated for 1 h at 37° C. and then washed four times with PBS-T. Peroxidase-conjugated rabbit anti-mouse IgM (optimal dilution 1:1000) IgG, IgG1, IgG2a, IgG2b antibodies (Zymed, San Francisco, Calif.) or IgG3 (optimal dilution 1:3000)(Rockland, Gilbertsville, Pa.) was added, and the plates were incubated for 1 h at 37° C. and washed three times with PBS-T. Orthophenylenediamine (0.5 mg/mL; Sigma-Aldrich) in 0.1 M citrate buffer (pH 5.6) containing 30% hydrogen peroxide was used as the enzyme substrate. The reaction was stopped with 2.5 N H2SO4, and the absorbance was determined at 490 nm using an automatic ELISA plate reader (Dynex Technologies MEI, Chantilly, Va., USA) with BIOLINX 2.22 software. Antibody titers are given as -log2 dilution×40. A positive titer was defined as 3 SD above the mean value of the negative control.

Results

The translation of innate immune response into antibody response is observed when adjuvants are co-administered to poor immunogenic vaccines. Adjuvants are substances capable of strengthen or augment the antibody or cellular immune response against an antigen. To determine whether PapMV is an adjuvant that can promote a long-lasting antibody response to other antigens, BALB/c mice were immunized with OVA or HEL either alone or together with the following adjuvants: PapMV, CFA, or LPS. The IgG antibody titer specific for OVA or HEL was measured by ELISA at the time points indicated (FIGS. 15A and B). PapMV adjuvant effect was observed on the total IgG response to OVA and HEL in immunized animals. An adjuvant effect induced by PapMV was observed for HEL on day 30 after immunization, when the antibody titer increased 8-fold compared with the antibody titer induced by HEL alone. This difference in antibody titer was maintained until day 120 but not on day 400 (FIG. 15A). Although LPS induced an adjuvant effect only in the first 30 days after immunization, CFA showed the strongest adjuvant effect from day 8 to the end of the experiment on day 400 after immunization (FIG. 15A). For immunization with OVA, the adjuvant effect on total IgG antibody titers was observed only until day 120 after the first immunization, after which antibody titer decreased with time and was 4-fold higher compared with OVA on day 400 (FIG. 15B). Further analysis was performed on day 20 to identify which IgG subclasses were induced by OVA and OVA coimmunized with adjuvants (where PapMV did not show an adjuvant effect on the total IgG response). PapMV, LPS, and CFA induced OVA-specific IgG2a and IgG2b antibody titers, whereas OVA alone induced only IgG1-specific antibody titers (FIG. 15C-E). No adjuvant effect for IgG1 was observed when OVA was coimmunized with any of the adjuvants used. These results show that PapMV, LPS, and CFA induce an adjuvant effect on the IgG subclass responses to OVA. Moreover, PapMV exhibits adjuvant properties that induce a long-lasting increase in specific antibody titers to model antigens. Taken together, these data suggest that PapMV has intrinsic adjuvant properties that may have mediated the translation of the innate response into the antigen-specific long-lasting antibody response observed.

### Example VI

### Purification of Salmonella typhi Porin Proteins

The following purification procedure was used for purification of OmpC and OmpF. The purification procedure is based on that described by Secundino et al. (2006), Immunology 117:59.

The two proteins were co-purified from Salmonella typhi. Individual purification of OmpC and OmpF was achieved using knock-out mutants of S. typhi in which either OmpC [STYC171 (OmpC⁻)] or OmpF [STYF302 (OmpF⁻)] open reading frames are interrupted. The procedure for purification of the individual proteins from the knock-out mutated forms of the bacteria was followed as for the co-purification. This procedure is outlined below.

The bacterial strain, Salmonella typhi 9,12,Vi:d (ATCC 9993) was grown in Minimal medium A supplemented with yeast extract, magnesium and glucose at 37° C., 200 rpm. The formula for 10L Minimal medium A supplemented with yeast extract, magnesium and glucose is: 5.0 g of dehydrated Na-Citrate (NaC₆H_{S}O₂:2H₂O), 31.0 g NaPO₄ monobasic (NaH₂PO₄), 70.0 g NaPO₄ dibasic (Na₂HPO₄), 10.0 g (NH₄)₂SO₄, 200 mL yeast extract solution 5% (15.0 g in 300 mL). 1.434L medium was distributed per 4L Erlenmeyer flask. Sterilization was performed at 121° C., 15 lbs pression/in², 15 min. o each flask was then added: 6.0 mL of sterile MgSO4 solution 25% and 60.0 mL of glucose solution 12.5%. The flask was inoculated with an overnight culture of S. typhi and when the OD₅₄₀ reached 1.0, incubation was stopped and the culture centrifuged at 7,500 rpm for 15 min at 4° C. The pellet was resuspended in 100 mL final of Tris-HCl pH 7.7 (6.0 g Tris-base/L) and the biomass was sonicated for 90 min on ice and then centrifuged at 7,500 rpm for 20 min at 4° C. To each 10mL of supernatant was added: 2.77 mL MgCl₂ 1M, 25 ml RNaseA (10,000 U/mL), 25 ml DNaseA (10,000 U/mL). The mixture was then incubated at 37° C. and 120 rpm for 30 min.

Porin extraction from the mixture was performed as follows:
1. Ultracentrifugation was performed at 45,000 rpm, 45 min, 4° C. and the pellet retained.
2. The pellet was resuspended in 10 mL Tris-HCl-SDS 2% followed by homogenisation.
3. An incubation step was performed at 32° C., 120 rpm, 30 min.
4. Ultracentrifugation followed at 40,000 rpm, 30 min, 20° C. and the pellet retained.
5. The pellet was resuspended in 5 mL Tris-HCl-SDS 2% followed by homogenisation.
6. An incubation step was performed at 32° C., 120 rpm, 30 min.
7. Ultracentrifugation followed at 40,000 rpm, 30 min, 20° C. and the pellet retained.
8. The pellet was resuspended in 20 mL Nikaido buffer-SDS 1% followed by homogenisation. [For 1L of Nikaido buffer: 6.0 g Tris-base, 10.0 g SDS, 23.4 g NaCl, 1.9 g EDTA was dissolved in water and the pH adjusted to pH 7.7. 0.5 mL β-mercaptoethanol solution was then added]
9. The mixture was incubated at 37° C., 120 rpm, 120 min.
10. Ultracentrifugation followed at 40,000 rpm, 45 min, 20° C. The supernatant, which contained the porin extract, was recovered.

The porins were purified from the supernatant using fast protein liquid chromatography (FPLC). 0.5× Nikaido buffer (see above) without β-mercaptoethanol was employed during the purification process. The proteins were separated using a Sephacryl S-200 (FPLC WATERS 650 E) with a Flux speed: 10 mL/min. The column was loaded with 22 mL of supernatant. Eluted fractions were monitored at 260 and 280 nm. The main peak, which contained the purified porins, was retained and stored at 4° C. The purified porins were stable for long period (over one year).

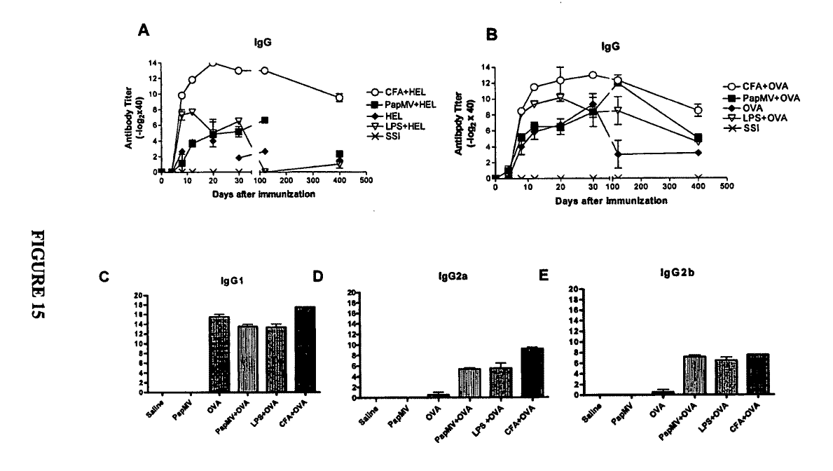

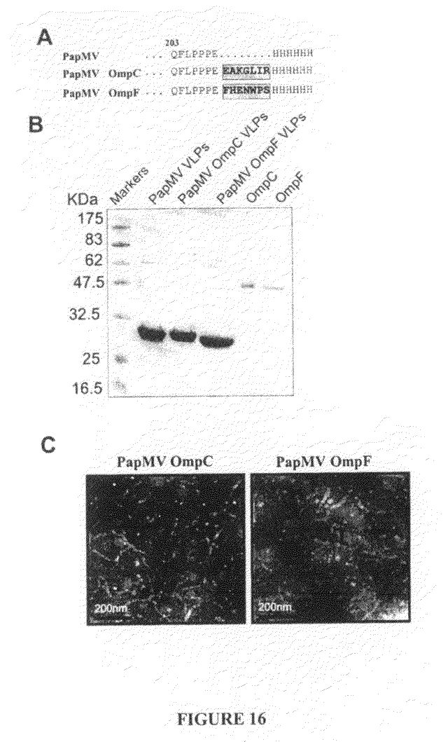

Results: FIG. 16B shows the SDS-PAGE profile of the porins, OmpC and OmpF, purified by the procedure described above.

### Example VII

### Production and Engineering of PapMV VLPs Comprising Affinity Peptides

Selection of Affinity Peptides

Specific peptides against purified OmpC and OmpF were selected using the Ph.D-7 Phage Display Peptide Library Kit (New England Biolabs, Inc.). The protocol followed was an in vitro selection process known as “panning,” which was conducted according to the manufacturer's protocol. Briefly, 2×10¹¹ phage were added to 10 ug of purified OmpC or OmpF bound to the base of the wells of an ELISA plate and the contents of the well gently mixed at room temperature for 1 hour. Unbound phage were eluted with 1 ml of 200 mM Glycine-HCl (pH 2.2), by incubating for 10 min at room temperature. To neutralize the supernatant, and to avoid killing the phage, 150 μl of 1M Tris-HCl (pH 9.1) was added. The eluted phage were then amplified and taken through additional binding/amplification cycles to enrich the pool in favour of binding sequences. The wash buffer contained 0.1% of Tween 20 for the first round of panning and was increased to 0.5% for subsequent rounds. Selected phage were amplified in E. coli ER2738 between each panning round. The cycle was repeated 3 times to select those peptides with the highest affinity for the respective porin proteins. The peptides thus identified are shown in Table 6.

TABLE 6
Sequence and Frequency of Occurrence
of OmpC and OmpF Affinity Peptides

| | Sequence | | |
|---|---|---|---|
| Target Protein | of Peptide | Frequency | SEQ ID NO |
| OmpC | SLSLIQT | 1/8 | 21 |
| OmpC | EAKGLIR | 6/8 | 22 |
| OmpC | TATYLLD | 1/8 | 23 |
| OmpF | FHENWPS | 3/5 | 24 |
| OmpF | FHEFWPT | 2/5 | 25 |

Engineering, Expression and Purification of the High Avidity PapMV VLPs Fused to the Selected Affinity Peptides

One affinity peptide was selected from those identified in the above panning process for each porin, OmpC and OmpF. The corresponding DNA sequence was cloned at the C-terminus of PapMV coat protein (CP). PapMV CP CPΔN5 (Tremblay, M-H., et al., 2006, FEBS J., 273:14-25; see Example I) was used as the template. The sequence encoding each selected peptide was introduced using PCR and cloned into the pET-3D expression vector (Stratagene, La Jolla, Calif.). In brief; the forward primer (SEQ ID NO:37; below) and the primer PapOmpC (SEQ ID NO:38; below) were used in the PCR reaction with the PapMV CP gene PapMV CP CPΔN5 as template.

Forward Primer:

[SEQ ID NO: 37]

5′-ATCGCCATGGCATCCACACCCAACATAGCCTTCCCCGCCATCACC-
3′
PapOmpC (Reverse Primer):

[SEQ ID NO: 38]

3′-GGTTAAGGAAGGTGGGGGGCTTCTCCGCTTCCCCAACTAAGCATGGT
AGTGGTAGTGGTAATCATTCCTAGGTGAC-5′

The resulting PCR fragment harbours a fusion of the peptide EAKGLIR at the C-terminus of the PapMV CP. Using the same approach, the forward primer (SEQ ID NO:37) and the primer PapOmpF (SEQ ID NO:41; below) were used to introduce a fusion of the peptide FHENWPS at the C-terminus of the PapMV CP by PCR.

PapOmpF (Reverse Primer):

[SEQ ID NO: 41]

3′-GGTTAAGGAAGGTGGGGGGCTTAAAGTACTCTTAACCGGAAGCGTGG
TAGTGGTAGTGGTAATCATTCCTAGGTGAC-5′.

The two respective PCR fragments were digested with the restriction enzymes NcoI and BamHI and cloned into the pET 3-D vector digested with the same enzymes. Clones were sequenced to verify that the peptides were in frame with the PapMV CP.

Engineered PapMV CPs comprising the affinity peptide were expressed in E. coli BL21 RIL as described previously (Tremblay, M-H., et al., 2006, FEBS J., 273:14-25; Secundino et al., 2006, Immunology 117:59). Briefly, the bacteria were lysed through a French Press and loaded onto a Ni²⁺ column, washed with 10 mM Tris-HCl 50 mM Imidazole 0.5% Triton X100 pH8, then with 10 mM Tris-HC1, 50 mM Imidazole, 1% Zwittergent pH8 to remove endotoxin contamination. The eluted proteins were subjected to high-speed centrifugation (100 000 g) for 120 min in a Beckman 50.2 TI rotor. The VLP pellet was resuspended in endotoxin-free PBS (Sigma). Proteins were filtered using 0.45 μM filters before use. The purity of the proteins was determined by SDS-PAGE. The amount of protein was evaluated using the BCA protein kit (Pierce). The level of LPS in the purified protein was evaluated with the Limulus test according to the manufacturer's instructions (Cambrex) and was below 0.005 endotoxin units (EU)/μg of protein.

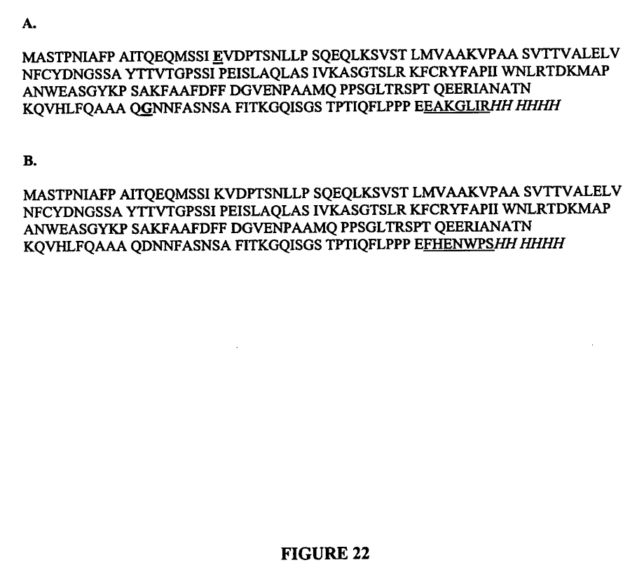

The sequences of the two PapMV coat proteins are shown in FIG. 22 (SEQ ID NO:42—PapMV coat protein including the OmpC affinity peptide (FIG. 22A), and SEQ ID NO:43—PapMV coat protein including the OmpF affinity peptide (FIG. 22B)). Two amino acid differences were observed in the coat protein sequence of the PapMV coat protein including the OmpC affinity peptide as compared to the wild-type (in bold and underlined in FIG. 22A), which were likely introduced during the PCR reaction.

ELISA

For each experiment, 10 μg of the respective target protein (OmpC or OmpF) was used to coat an ELISA plate. Increasing amounts of the PapMV VLPS were used for the binding assay. The affinity of the VLPs for their target was revealed using polyclonal mouse antibodies directed to the PapMV CP and a secondary anti-mouse antibody coupled to peroxidase.

Results

Selection of Affinity Peptides

Phage display was used to select specific peptides binding to OmpC or OmpF. Eight phage that bound to OmpC and five phage that bound to OmpF were sequenced. The sequences and frequency of occurrence of these peptides is show in Table 6. The peptide EAKGLIR [SEQ ID NO:22] showed the highest frequency and, therefore, was selected as the affinity peptide to OmpC. The peptide FHENWPS [SEQ ID NO:23] was the most frequent in the OmpF screening and was, therefore, selected as the affinity peptide to OmpF. Interestingly, both affinity peptides to OmpF were homologous since 5 out of 7 amino acids were identical and found in the same position in the affinity peptides.

Synthesis of High Avidity PapMV VLPs

The peptide sequences EAKGLIR [SEQ ID NO:22] and FHENWPS [SEQ ID NO:23] were fused at the C-terminus of the PapMV coat protein (FIG. 16A). The fusion peptide was followed by a 6xH tag to facilitate the purification process (Tremblay, M-H., et al., 2006, ibid.). The recombinant constructs were expressed in E. coli and purified by affinity chromatography on a Ni²⁺ column. The proteins were eluted using 500 mM imidazole, dialysed and ultracentrifuged at 100,000 g to pellet the VLPs (FIG. 16B). Electron microscopy (EM) observations confirmed that the addition of the peptides at the C-terminus of the PapMV CP did not affect the ability of the protein to self-assemble into VLPs (FIG. 16C). The lengths of the VLPs are variable, with a size range of 201±80 nm. A 201 nm length protein represents 560 copies of the CP presenting the peptide in a repetitive fashion.

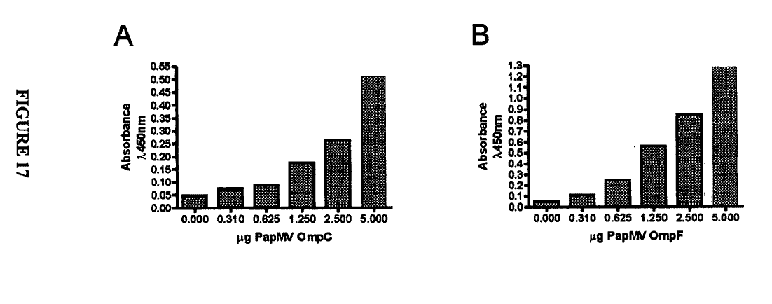

The high avidity of each of the PapMV VLPs to their respective antigen was shown by an ELISA-type binding assay. For both VLPs, binding to their respective antigen was clearly demonstrated and increased with the amount of VLPs used in the assay (FIG. 17A-B). It was, therefore, assumed that PapMV VLPs will bind to the cognate antigen to form a complex when mixed in a 1:1 ratio in solution.

### Example VIII

### Immunization Against Salmonella typhi with High Avidity PapMV VLPs

Mice

Female BALB/c mice 6-8 weeks old (Harlan, Mexico or Charles River, Canada) were used and kept in the animal facilities of the Experimental Medicine Department, Medicine Faculty, National Autonomous University of Mexico (UNAM) or at the animal facilities from Centre Hospitalier de l'Université Laval.

Challenge Assay

Mice (10 per group) were immunized intraperitoneally (i.p) (day 0) in the absence of external adjuvant with 10 μg OmpC, 10 μg OmpC+10 μg PapOmpC, 10 μg OmpF, 10 μg OmpF+10 μg PapOmpF, 10 μg PapOmpC, 10 μg PapOmpF or saline (SSI). On day 15, mice received a boost i.p with 10 μg OmpC or 10 μg OmpF, respectively, without adjuvant. On day 25 or 140 the mice were challenged i.p with 100 or 500 LD₅₀ of Salmonella typhi (ATCC 9993) resuspended in 500 μL TE buffer (50 mM Tris, pH 7.2, 5 mM EDTA) containing 5% gastric mucin (Sigma). Protection was defined as the percentage survival 10 days following the challenge. 1 LD₅₀ was determined at 90 000 CFU.

Immunizations

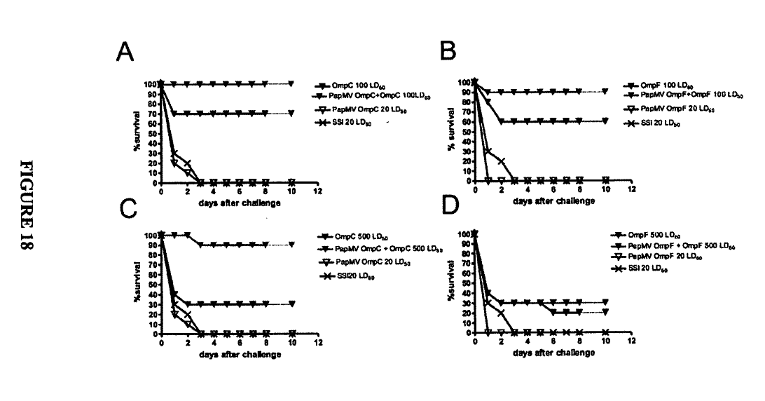

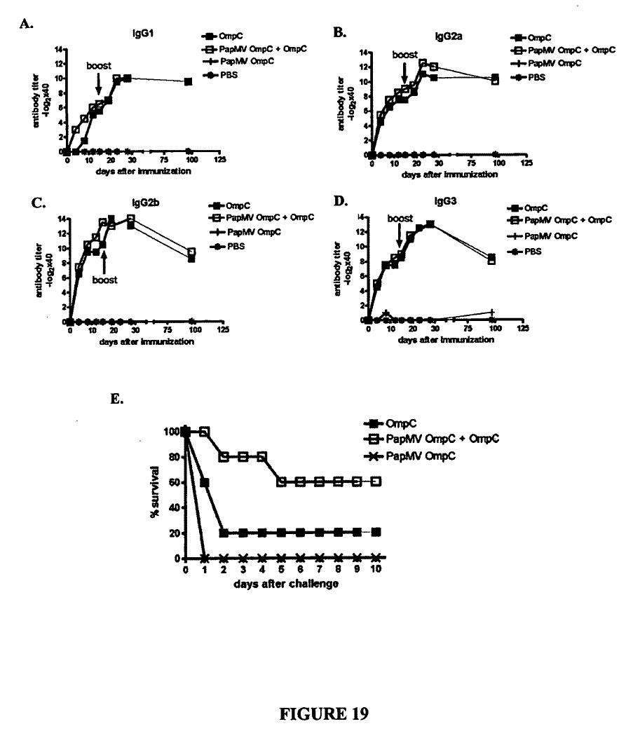

Groups of 5 mice were immunized (day 0) intraperitoneally (i.p) in the absence of external adjuvant with 10 μg OmpC, 10 μg OmpC+10 μg PapOmpC, 10 μg PapOmpC or isotonic saline solution (ISS). On day 15, mice received a boost i.p with 10 μg OmpC without adjuvant. Blood samples were collected from the jugular vein at various times as indicated in FIGS. 18 and 19. Individual serum samples were stored at −20° C. until analysis.

ELISA

High binding 96-well polystyrene plates (Nunc) were coated with 10 μg/mL of OmpC in 0.1M carbonate-bicarbonate buffer ph 9.5. Plates were incubated for 1 hour at 37° C. followed by overnight at 4° C. Plates were washed four times with distilled H₂O-0.1% Tween 20. Non-specific binding was blocked with blocking buffer (PBS pH 7.4 -2% BSA (Sigma)) for 1 hour at 37° C. After washing, pooled mice sera were diluted 1:40 in blocking buffer and two-fold serial dilutions were added to the wells. Plates were incubated 1.5 hours at 37° C., followed by four washes: HRP-conjugated goat anti-mouse IgG₁, IgG₂ₐ, IgG_{2b} (Jackson Immunochemicals) or IgG₃ (Rockland) (1:10 000) was added and incubated 1 hour at 37° C. followed by four washes. As the detection system, TMB peroxidase substrate (Fitzgerald) was used. After incubation in the dark for 10 minutes at 37° C. the reaction was stopped with 2.5N H₂SO₄ and the absorbance was determined at 450 nm using an automatic ELISA plate reader. Antibody titers are given as -log₂ dilution X40. Positive titers were defined as 3 SD above the mean values of the negative controls.

Passive Immunization and Challenge

Groups of 5 mice were immunized i.p (day 0) in the absence of external adjuvant with 10 μg OmpC, 10 μg OmpC+10 μg PapMV OmpC, 10 μg PapMV OmpC or isotonic saline solution (SSI). On day 15, mice received a boost i.p with 10 μg OmpC without adjuvant. Cardiac puncture was performed on day 23 and serum samples from each group were pooled and stored at −20° C. Naïve mice (5 per group) received i.p 200 μL of the pooled complement-inactivated immune sera. Three hours after transference mice were challenged with 100 LD₅₀ of Salmonella typhi resuspended in mucin, as described above. Protection was defined as the percentage survival 10 days following the challenge.

Results

PapMV VLPs Improve the Protection Capacity of Porins

The purified proteins OmpC and OmpF were previously shown to provide protection against S. typhi challenge in mice, with OmpC alone providing 60% protection against 100 LD₅₀ (Secundino et al., 2006, Immunology 117:59). To improve the immunogenicity of each of the porins, OmpC and OmpF, respectively, vaccine formulations comprising PapMV VLPs were prepared. Two different preparations, PapMV OmpC VLPs+OmpC and PapMV OmpF+OmpF, were tested in mice for their capacity to protect mice toward 100 and 500 LD₅₀ of S. typhi and the results compared with those obtained with mice immunized with OmpC or OmpF alone. The ratio between the PapMV VLPs and their respective porin was maintained at 1:1.

Addition of PapMV OmpC VLPs to OmpC improved the protection capacity of OmpC from 70% to 100% with a challenge of 100 LD50 of S. typhi (FIG. 18A). This improvement of the protection efficacy was even greater when mice were challenged with 500 LD₅₀, with the protection observed increasing from 30% to 90% when OmpC was combined with PapMV OmpC VLPs (FIG. 18C). Similarly, PapMV OmpF VLPs improved the protection capacity of OmpF from 60% to 90% with a challenge of 100 LD₅₀ of S. typhi (FIG. 18B), however, only a minor difference was observed when the challenge was conducted with 500 LD₅₀ of S. typhi (FIG. 18D). The results suggest that OmpC is a better antigen than OmpF for protection against S. typhi challenge. In both cases, PapMV VLPs considerably improved the protective capacity of the porins.

To determine if PapMV VLPs improved antibody titers to OmpC, the IgG titers of mice vaccinated with 10 μg OmpC or with the conjugated vaccine containing 10 μg OmpC and 10 μg PapMV OmpC VLPs were measured. No significant difference was found in the titers of the different IgG isotypes IgG1, IgG2a, IgG2b and IgG3 with either treatment (FIG. 19A-D) suggesting that the improvement of the protection observed with PapMV VLPs may be related to an improvement in the CTL response and/or in the binding efficacy of the antibodies in neutralising S. typhi infection, rather than an increase of production of antibodies per se.

Vaccination with the PapMV VLPs Improves the Memory Response to the Porins

To evaluate the memory response of the vaccine preparation comprising the PapMV VLPs in combination with OmpC, mice were immunized twice at two-week intervals with either OmpC alone, or with the vaccine preparation comprising PapMV OmpC VLPs and OmpC, followed with a boost at day 15 with OmpC alone. At day 140, the mice were challenged with 100 LD₅₀ of S. typhi. The results clearly show that priming with the vaccine preparation comprising PapMV OmpC VLPs and OmpC significantly improved (3 times improvement) the protection capacity of vaccinated mice (FIG. 19E). This experiment thus demonstrates that PapMV VLPs not only improve the protection of mice to S. typhi challenge, but also provide a better memory response.

### Example IX

### Protective Capacity of a Combination of PapMV and OmpC Against S. typhi

PapMV was purified as described in Example I.

Protection Assay

BALB/c mice (groups of 10) were immunized i.p. on day 0 with 10 μg of OmpC or 10 μg of OmpC that had been incubated previously for 1 h at 4° C. with 30 μg of PapMV. A boost on day 15 was performed with 10 μg of OmpC alone. Control mice were injected with saline only. On day 21, the mice were challenged with 100 and 500 LD₅₀ of S. typhi (STYC302 DompF strain) suspended in 5% mucin (as described above) and the survival rate was monitored for 10 days after the challenge, as described previously (Isibasi et al., 1992, Vaccine 10:811-813; Isibasi et al., 1988, Infect. Immun. 56:2953-2959).

Results

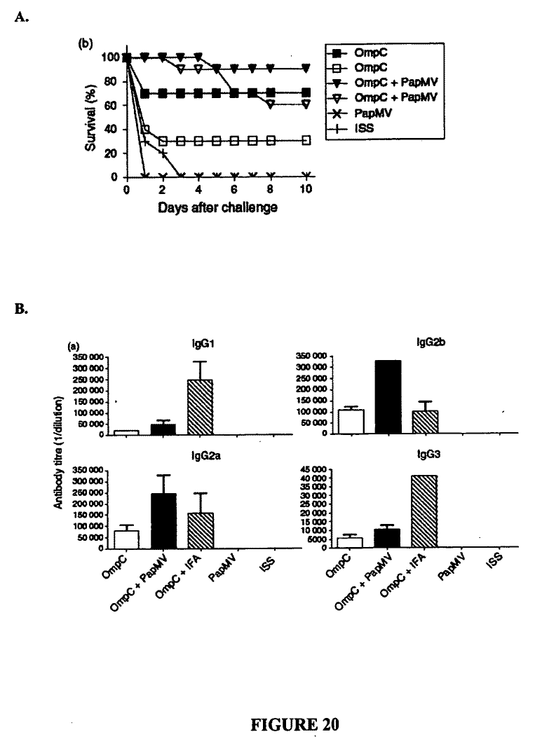

To test the adjuvant capacity of PapMV virus isolated from infected plants in increasing the protection privided by OmpC, mice immunized with OmpC and mice immunized with OmpC mixed with PapMV purified virus and subsequently challenged with S. typhi were compared. A survival rate 30% higher after challenge with either 100 LD₅₀ and 500 LD₅₀ of S. typhi was observed when OmpC mixed with PapMV purified virus was employed as compared to OmpC alone (FIG. 20A). No protection was observed in mice immunized only with PapMV on days 0 and 15 and challenged on day 21 (FIG. 21A), nor in mice immunized with PapMV and challenged 24 hr later. These data reject the idea that the protection observed is the result of enhanced inflammation induced by PapMV. To test if increased protection correlates with an increase in the antibody titre specific for OmpC, the PapMV adjuvant effect on the OmpC-specific antibody titres was measured. PapMV co-immunization with OmpC induced an increase in the anti-OmpC IgG1, IgG2a, IgG2b and IgG3 titres (FIG. 21B).These results further corroborate that the adjuvant properties of PapMV potentiate both innate and adaptive immune responses elicited by OmpC to achieve protection against S. typhi challenge. Co-administration of PapMV and S. typhi OmpC porin can thus be seen to increase the protective capacity against S. typhi challenge.

The results of the experiments outlined in Examples V-IX indicate that PapMV has intrinsic adjuvant properties that can induce the switch of antigen-specific immunoglobulins, provide a sustained long lasting antibody response to model antigens, and increase the protective capacity of OmpC or OmpF alone. These data indicate that PapMV and PapMV VLPs potentiate the translation of innate and adaptive immune responses elicited by OmpC porin into protection against S. typhi challenge.

### Example X

### Immunogenic Effect of PapMV VLP Harbouring S. typhi Porin Epitope

Two small epitopes corresponding to loop 6 and 7 of the S. typhi porin that are exposed on the surface of the bacterium have been shown to be involved in protective mechanisms elicited by immunization with porins (Panigua-Solis et al., 1995, Immunol Infect. Dis. 5:244-249). These regions are only present in S. typhi porins, therefore, no crossreactivity with porins from other gram negative bacteria has been found. Thus, porin fragments which include these epitopes represent excellent candidate antigens for the development of a recombinant subunit vaccine utilizing PapMV VLPs as an adjuvant-carrier.

Production of Recombinant PapMV VLPs

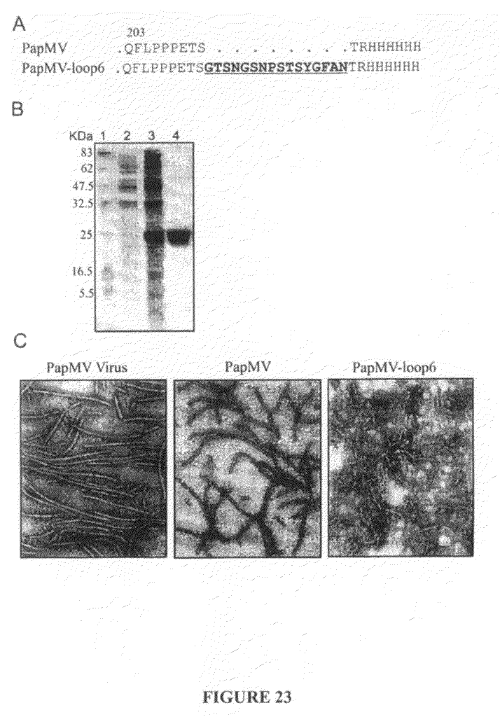

PapMV coat protein was engineered to include at its C-terminus the amino acid sequence GTSNGSNPSTSYGFAN [SEQ ID NO:50], which includes the immunodominant loop 6 epitope from S. typhi porin (see FIG. 23A). The recombinant protein was expressed in E. coli (see FIG. 23B) using methods as described in the previous Examples. In brief, the forward primer (SEQ ID NO:44; below) and the reverse primer (SEQ ID NO:45; below) were used in the PCR reaction with the PapMV CP CPΔN5 gene as template.

Forward primer:

[SEQ ID NO: 44]

5′-CTAGTGGTACTTCTAACGGTTCTAACCCGTCTACTTCTTACGGTTTC
GCGAACA-3′
Reverse Primer:

[SEQ ID NO: 45]

5′-CTAGTGTTCGCGAAACCGTAAGAAGTAGACGGGTTAGAACCGTTAGA
AGTACCA-3′

The recombinant proteins were purified from E. coli by Ni²⁺ affinity tag chromatography, as described in the preceding examples, and were shown to self-assemble into VLPs that are similar similar to the native virus particle purified from infected plant leaves (see FIG. 23C).

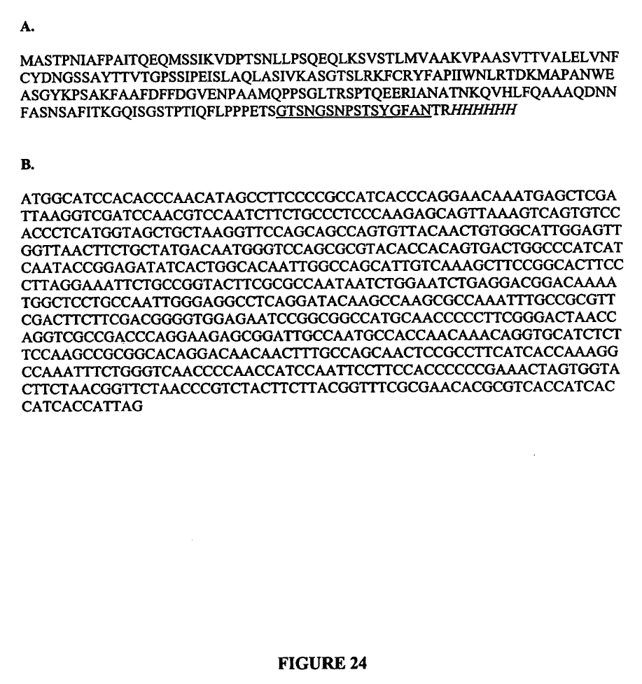

The amino acid sequence of the PapMV coat protein comprising the loop 6 peptide (PapMV-loop6) is shown in FIG. 24A (SEQ ID NO:46), and the nucleotide sequence encoding the PapMV-loop6 protein is shown in FIG. 24B (SEQ ID NO:47).

Immune Response to the PapMV-loop6 in Balb/C Mice

Female BALB/c mice 6-8 weeks old (Harlan, Mexico or Charles River, Canada) were used and kept in the animal facilities of the animal facilities from Centre Hospitalier de l'Université Laval. Mice were immunized by the subcutaneous route (s.c.) (day 0) in the absence of external adjuvant with 100 μg PapMV-loop6. On day 15, mice received a boost s.c. with 100 μg PapMV-loop6. On day 28 the final bleed was collected and the immune responses were determined by standard ELISA using the loop6 synthetic peptide and purified OmpC protein.

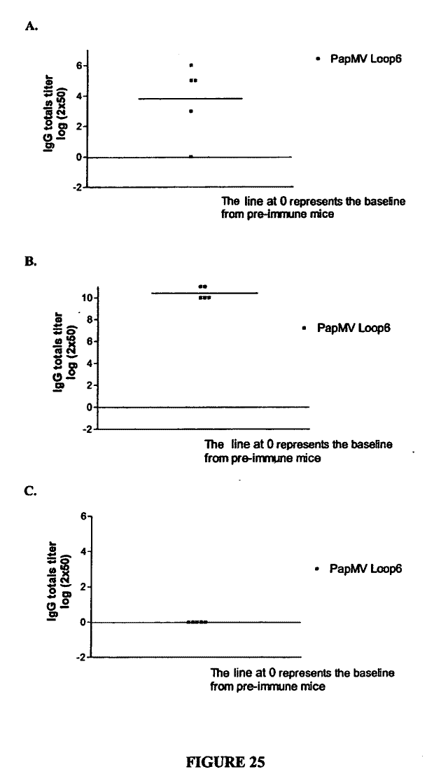

The results confirm that the PapMV platform is highly immunogenic as it triggered the production of loop6 specific antibodies in 4 mice out of 5 (FIG. 25A). Although the PapMV-loop6 VLPs triggered a large number of antibodies to the PapMV platform in the 5 treated mice (FIG. 25B), the same sera did not show a reaction against the purified OmpC protein (FIG. 25C). This result suggests that the structure of the loop6 peptide in the PapMV CP fusion may adopt a slightly different conformation to that of the peptide in' its native environment within the OmpC protein.

In order to allow the loop6 peptide sequence to adopt its native conformation when fused to the PapMV CP, the above sequence (SEQ ID NO:50) will be cloned into an internal loop between the residues 49-52 of the PapMV CP. This loop was identified for this purpose using bio-informatic techniques.

### Example XI

### Protective Capacity of PapMV VLPs with S. typhi OmpF

The following proteins, as described in Examples VII, VIII and IX, were used in this experiment:
PapMV CPΔN5 VLPs (“PapMV”) (see FIG. 1C).
PapMV OmpF VLPs that comprise the affinity peptide for S. typhi OmpF (see FIG. 22B)
OmpF (see FIG. 21B)

Balb/C mice, 10 mice per group, were immunized intraperitonally (I.P.) at day 0 as follows:

Group 1-10 μg OmpF

Group 2-10 μg OmpF+10 μg PapMV

Group 3-10 μg OmpF+10 μg PapMV OmpF

Group 4-10 μg PapMV

Group 5-10 μg PapMV OmpF

A second immunization (Boost) was performed at day 15 using 10 μg OmpF in groups 1, 2 and 3. Group 4 was boosted with 10 μg PapMV, and group 5 was boosted with 10 μg PapMV OmpF. Challenge with S. typhi was performed on day 21. It was established experimentally that 90 000 CFU of S. typhi in mucin correspond to 1 LD₅₀. All mice were sacrificed at day 31.

Results

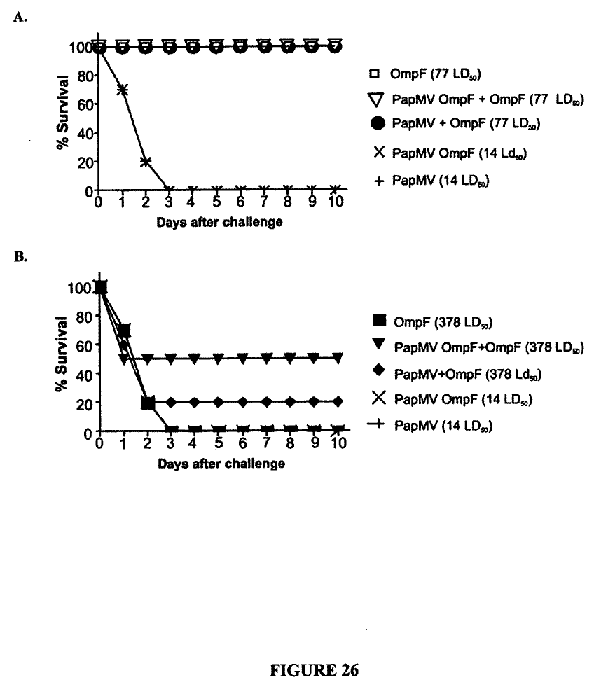

At 77 LD₅₀ (FIG. 26A), all preparations containing OmpF provided 100% protection. As expected, the mice vaccinated with preparations that did not contain OmpF all died within a few days of challenge with doses as low as 14 LD₅₀. A difference was observed, however, between the OmpF containing preparation when the mice were challenged with a higher dose of S. typhi (378 LD₅₀). As shown in FIG. 26B, the most effective preparation at this dose was the combination of PapMV OmpF+OmpF in the first immunisation (Group 3) and a boost with OmpF alone. It is likely that the high avidity of the PapMV OmpF VLPs for their target, OmpF, improved the protection capacity as compared to the use of PapMV alone as an adjuvant to OmpF.

This data is consistent with that shown in Example VIII and confirms that PapMV OmpF improves the protection capacity of OmpF to a challenge with S. typhi. Furthermore, the data show that PapMV OmpF is a better adjuvant of OmpF than PapMV VLPs without an affinity peptide. As noted above, it is likely that a stronger binding of PapMV OmpF VLPs to OmpF promotes formation of a V LP-OmpF complex, which in turn maximises the adjuvant capacity of the VLP molecule.

### Example XII

### Protective Capacity of PapMV VLPs with S. typhi OmpC

The following proteins, as described in Examples VII, VIII and IX, were used in this experiment:
PapMV CPΔN5 VLPs (“PapMV”) (see FIG. 1C).
OmpC (see FIG. 21A)

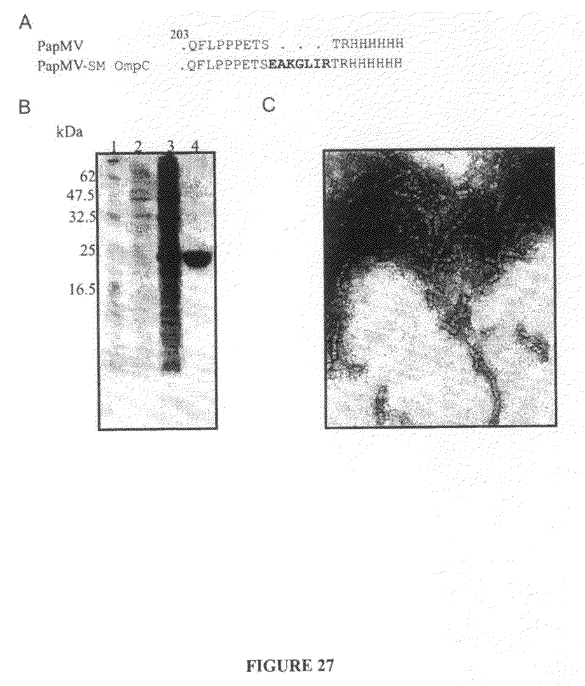

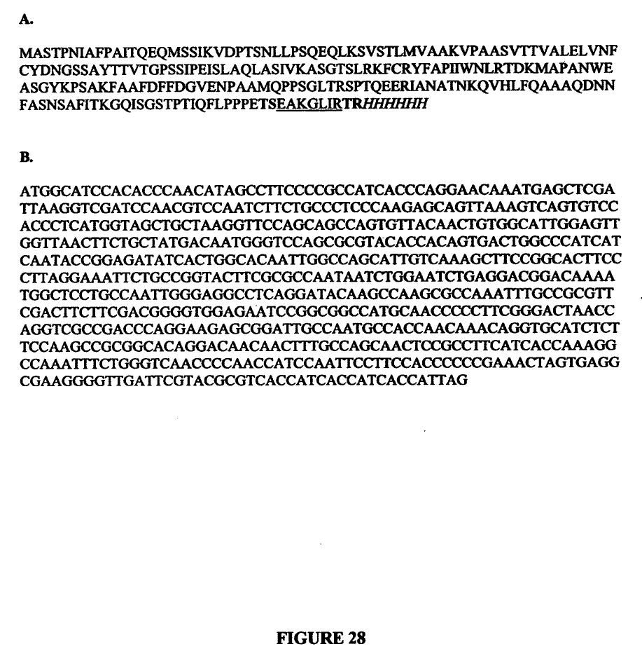

A further PapMV CP fusion comprising the OmpC affinity peptide EAKGLIR [SEQ ID NO:22] was constructed that included an additional 4 amino acids, 2 on each side of the affinity peptide (TR on the N-terminal side and TS on the C-terminal side, as shown in FIG. 27A). These amino acids are the result of the presence of the restriction sites SpeI-MluI that were used for the cloning of the affinity peptide in fusion with PapMV CP. The construct was designated “PapMV SM OmpC.” The complete amino acid sequence for the PapMV SM OmpC protein is provided in FIG. 28A (SEQ ID NO:48) and the nucleotide sequence encoding the PapMV SM OmpC protein is provided in FIG. 28B (SEQ ID NO:49).

The PapMV SM OmpC protein was purified as described in the preceding Examples (see FIG. 27B). The purified proteins showed VLP formation by electron microscopy, as expected (FIG. 27C).

Immunizations

Balb/C mice, 10 mice per group, were immunised intraperitonally (I.P.) at day 0 as follows:

Group 1-10 μg OmpC

Group 2-10 μg OmpC+10 μg PapMV

Group 3-10 μg OmpC+10 μg PapMV OmpC

Group 4-10 μg PapMV

Group 5-10 μg PapMV OmpC

A second immunization (Boost) was performed at day 15 using 10 μg OmpC in groups 1, 2 and 3. Group 4 was boosted with 10 μg PapMV, and group 5 was boosted with 10 μg PapMV OmpC. Challenge with S. typhi was performed on day 21. It was established experimentally that 90 000 CFU of S. typhi in mucin correspond to 1 LD₅₀. All mice were sacrificed at day 31.

Results

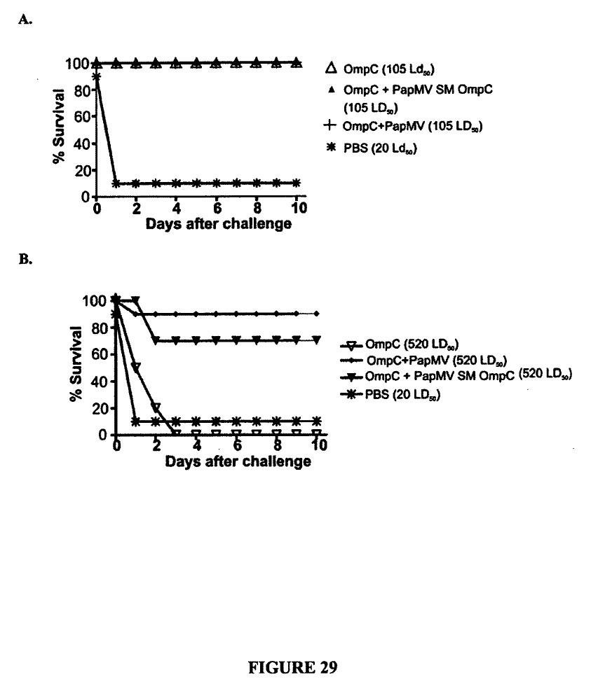

At 105 LD₅₀ (FIG. 29A), all the preparations containing OmpC provided 100% protection. As expected, the mice vaccinated with preparations that did not contain OmpC all died within a few days of challenge with doses as low as 20 LD₅₀. A difference was observed, however, between the OmpC-containing preparations when the mice were challenged with a higher dose of S. typhi (520 LD₅₀). As can be seen from FIG. 29B, the most effective preparation at this dose was the combination of PapMV+OmpC in the first immunisation (Group 2) and a boost with OmpC alone. The preparation comprising PapMV SM OmpC VLPs conjugated to OmpC (Group 3) also performed well but appears to be slightly less effective than the PapMV+OmpC combination. However, whether or not the difference in efficacy was sufficient to be statistically significant could not be determined in this experiment.

In conclusion, as shown in Examples VII, XI and XII, the recombinant PapMV VLPs, with or without affinity peptides, are capable of acting as strong adjuvants that improve the protection capacity of two different antigens of S. typhi; OmpC and OmpF respectively.

### Example XIII

### Stability of S. typhi Porins

The stability of S. typhi purified porins was assessed by standard SDS-PAGE. Two samples of porins were used. The first sample included both OmpC and OmpF purified proteins (lot ISIPOR) and was prepared on Nov. 9, 1999 (this preparation is identical to that shown in FIG. 1(b) of Salazar-Gonzales et al., 2004, Immunology Letters 93. The second sample included purified OmpC protein only (lot-1239) and was prepared on Apr. 30, 2007.

Both samples were stored in PBS solution pH 7.4±0.1 at 4±3° C. in the dark. SDS-PAGE was conduted using the following conditions:
12% acrylamide.
All samples were denatured by addition of (3-mercaptoethanol and heating at 95° C. for 5 min before loading onto the gel.

The results are shown in FIG. 30. As can be seen from this figure, both porin preparations are very stable. Isipor appeared to be very stable for more than 9 years (from 1999 to 2008) as no signs of degradation were visible by SDS-PAGE (cf. FIGS. 30A and 30B). The purified OmpC also demonstrated high stability with the protein remaining intact after 9 months of storage (cf. left and right hand panels of FIG. 30C).

### Example XIV

### PapMV Induces Upregulation of Activation Markers on DCs, Macrophages and B Cells In Vivo

PapMV was purified as described in Example I.

In vivo DC, macrophage and B-lymphocyte activation assay

BALB/c mice (7 weeks of age) were immunized i.p. with 30 μg of PapMV and 50 μg of Poly I:C suspended respectively in 500 μl of saline. Saline (500 μl) was used as a control, and 24 hr after immunization lymph nodes and spleens were obtained. Cell suspensions were prepared from these organs, and flow cytometry staining was performed using phycoerythrin (PE)-CD11c, allophycocyanin-CD11b, B220-peridinin chlorophyll protein (PerCP), FITC-CD80, FITC-CD86, FITC-CD40, FITC-CD69 and FITC-major histocompatibility complex class II (MHC class II) antibodies (all purchased from BD Pharmingen).

Results

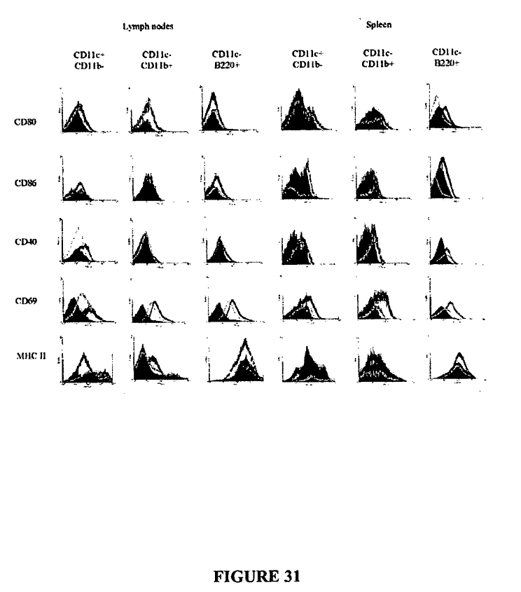

It is believed that PapMV is recognized by the immune system as a pathogen-associated molecular pattern (PAMP) and, as such, it should drive the maturation of immature DC or activate APC, as do other pattern recognition receptor (PRR) agonists. To characterize the capacity of PapMV to stimulate APC in vivo, lymph nodes and spleens of mice immunized with PapMV were isolated and analysed for up-regulation of co-stimulatory and activation molecules (CD80, CD86, CD40, CD69 and MHC class II). As shown in FIG. 31, PapMV induced the up-regulation of CD80 and CD69 in lymph node (LN) DC (CD11c+ CD11b−) and CD40 and CD69 in spleen DC. For macrophages (CD11c− CD11b+), up-regulation of CD80 and CD69 was observed in LN, whereas CD40 and CD69 up-regulation was observed in the spleen. CD69 was up-regulated in LN B lymphocytes (CD11c− B220+), and CD40 and CD69 were up-regulated in splenic B cells. The TLR-3 agonist, Poly I:C, induced the upregulation of the markers tested, although the profile observed was markedly different from that induced by PapMV (FIG. 31).

Antigen presentation by APCs to T cells has shown the importance of the innate immune system to activate the adaptive immune system. APCs loaded with PapMV were observed to efficiently induced antibody responses. These data, and the effects elicited by PapMV on APCs (FIG. 31), suggest that PapMV-stimulated APCs provide the antigen and the cytokine environment needed to promote an efficient T-cell-dependent antibody response, and this is likely to be the mechanism involved in translating intrinsic adjuvant signals delivered by PapMV into the long-lasting antibody response that has been observed. PapMV is likely sensed by the immune system both as PAMP and as antigen (Pamptigen), simultaneously activating innate and adaptive immune responses, which would favour the induction of a memory compartment. Pamptigens could also bind both BCRs and PRRs, such as TLRs, on specific B cells, leading to the differentiation of antibody-secreting cells or to the generation of memory B cells. A Pamptigen could reduce the antigen threshold required to activate the immune response. Accordingly, small amounts of the persistent antigen could maintain a high antibody titre.

The strong immunogenicity and intrinsic adjuvant properties of PapMV translate into a specific long-lasting antibody response to both PapMV and to model coimmunized antigens (see Example IX). As PapMV can be produced using inexpensive procedures and is stable at room temperature, it is well suited for development of new adjuvant and vaccine platforms to induce long-lasting immunity.

Although the invention has been described with reference to certain specific embodiments, various modifications thereof will be apparent to those skilled in the art without departing from the spirit and scope of the invention. All such modifications as would be apparent to one skilled in the art are intended to be included within the scope of the following claims.

1. An antigen-presenting system, comprising one or more enterobacterial antigens in combination with a papaya mosaic virus (PapMV) or a virus-like particle (VLP) derived from a PapMV coat protein, wherein the PapMV or VLP is capable of potentiating an immune response against said one or more enterobacterial antigens.

2. The antigen-presenting system of claim 1, wherein said VLP is derived from a modified PapMV coat protein, the modified PapMV coat protein being capable of multimerization to form the VLP.

3. The antigen-presenting system of claim 2, wherein the antigen-presenting system comprises a VLP comprising an amino acid sequence substantially identical to the sequence as set forth in SEQ ID NO:3.

4. The antigen-presenting system of claim 1, wherein the one or more antigens are conjugated to the coat protein of the PapMV or VLP.

5. The antigen-presenting system of of claims claim 1, wherein the one or more antigens are not conjugated to the PapMV or VLP.

6. The antigen-presenting system of claim 4, wherein the one or more antigens are genetically fused to the coat protein.

7. The antigen-presenting system of claim 4, wherein the one or more antigens are attached by affinity binding to the coat protein.

8. The antigen-presenting system of claim 7, wherein the affinity binding is through an affinity peptide genetically fused to the coat protein, the affinity peptide capable of binding the one or more antigens.

9. The antigen-presenting system of claim 1, wherein the one or more antigens are porin proteins or antigenic fragments thereof

10. The antigen-presenting system of claim 1, wherein the one or more antigens are selected from the group of: OmpC, fragments of OmpC, OmpF and fragments of OmpF.

11. The antigen-presenting system of claim 1, wherein the one or more antigens are Salmonella typhi antigens.

12. The antigen presenting system of claim 1, wherein the antigen-presenting system comprises a VLP comprising an amino acid sequence substantially identical to the sequence as set forth in SEQ ID NO:42, SEQ ID NO:43 or SEQ ID NO:48.

13. A vaccine composition, comprising:
the antigen-presenting system of claim 1, and
a pharmaceutically acceptable carrier.

14. A polypeptide, comprising a papaya mosaic virus coat protein fused to one or more affinity peptides capable of binding an enterobacterial antigen.

15. The polypeptide of claim 14, wherein the antigen is a Salmonella typhi antigen.

16. The polypeptide of claim 14, wherein the one or more affinity peptides each comprise a sequence selected from the sequences as set forth in SEQ ID NO:21, SEQ ID NO:22, SEQ ID NO:23, SEQ ID NO:24 and SEQ ID NO:25.

17. The polypeptide of claim 14, wherein the polypeptide comprises an amino acid sequence substantially identical to the sequence as set forth in SEQ ID NO:42, SEQ ID NO:43 or SEQ ID NO:48.

18. A polypeptid, comprising a papaya mosaic virus coat protein fused to one or more enterobacterial antigens selected from the group of: OmpC, fragments of OmpC, OmpF and fragments of OmpF.

19. The polypeptide of claim 18, wherein the one or more antigens are Salmonella typhi antigens.

20. The polypeptide of claim 18, wherein the one or more antigens are fused at the C-terminus or within an internal loop of the PapMV coat protein.

21. A polynucleotide encoding the polypeptide of claim 14.

22.-31. (canceled)

32. A vaccine for immunizing an animal against infection with Salmonella typhi, the vaccine comprising an antigen-presenting system comprising an antigen derived from Salmonella typhi OmpC or OmpF in combination with a papaya mosaic virus (PapMV) or a VLP derived from a PapMV coat protein, wherein the PapMV or VLP is capable of potentiating an immune response against the antigen in the animal.

33. The vaccine of claim 32, wherein the antigen-presenting system comprises a VLP comprising an amino acid sequence substantially identical to the sequence as set forth in SEQ ID NO:42, SEQ ID NO:43 or SEQ ID NO:48.

34. A method of inducing an immune response against an enterobacterium in an animal, the method comprising administering to the animal an effective amount of the antigen presenting system of claim 1.

35. The method of claim 34, wherein the VLP is derived from a modified PapMV coat protein, the modified PapMV coat protein being capable of multimerization to form the VLP.

36. The method of claim 35, wherein the antigen-presenting system comprises a VLP comprising an amino acid sequence substantially identical to the sequence as set forth in SEQ ID NO:3.

37. The method of claim 34, wherein the one or more antigens are conjugated to the coat protein of the PapMV or VLP.

38. The method of claim 34, wherein the one or more antigens are not conjugated to the PapMV or VLP.

39. The method of claim 37, wherein the one or more antigens are genetically fused to the coat protein.

40. The method of claim 37, wherein the one or more antigens are attached by affinity binding to the coat protein.

41. The method of claim 40, wherein the affinity binding is through an affinity peptide genetically fused to the coat protein, the affinity peptide capable of binding the one or more antigens.

42. The method of claim 34, wherein the one or more antigens are porin proteins or antigenic fragments thereof.

43. The method of claim 34, wherein the one or more antigens are selected from the group of: OmpC, fragments of OmpC, OmpF and fragments of OmpF.

44. The method of claim 34, wherein the one or more antigens are Salmonella typhi antigens.

45. The method of claim 34, wherein the antigen-presenting system comprises a VLP comprising an amino acid sequence substantially identical to the sequence as set forth in SEQ ID NO:42, SEQ ID NO:43 or SEQ ID NO:48.

46. The method of claim 34, wherein the enterobacterium is Salmonella typhi.

47. The method of claim 34, wherein the animal is a human.

48. The method of claim 34, wherein the animal is a non-human animal.

49. A polynucleotide encoding the polypeptide of claim 18.
